SEQUENCE LISTING (16 sequences)

SEQ ID NO: 1 - DNA, 1156 bp, Homo sapiens
  CDS (1)..(849)
gatgtggcagtggatgggcagaactggacgtttgcttttgacttctccttcctgagccaacaagaggatctggcatgggctgagctccggctgcagctgtccagccctgtggacctccccactgagggctcacttgccattgagattttccaccagccaaagcccgacacagagcaggcttcagacagctgcttagagcggtttcagatggacctattcactgtcactttgtcccaggtcaccttttccttgggcagcatggttttggaggtgaccaggcctctctccaagtggctgaagcgccctggggccctggagaagcagatgtccagggtagctggagagtgctggccgcggccccccacaccgcctgccaccaatgtgctccttatgctctactccaacctctcgcaggagcagaggcagctgggtgggtccaccttgctgtgggaagccgagagctcctggcgggcccaggagggacagctgtcctgggagtggggcaagaggcaccgtcgacatcacttgccagacagaagtcaactgtgtcggaaggtcaagttccaggtggacttcaacctgatcggatggggctcctggatcatctaccccaagcagtacaacgcctatcgctgtgagggcgagtgtcctaatcctgttggggaggagtttcatccgaccaaccatgcatacatccagagtctgctgaaacgttaccagccccaccgagtcccttccacttgttgtgccccagtgaagaccaagccgctgagcatgctgtatgtggataatggcagagtgctcctagatcaccataaagacatgatcgtggaagaatgtgggtgcctctgatgacatcctggagggagactggatttgcctgcactctggaaggctgggaaactcctggaagacatgataaccatctaatccagtaaggagaaacagagaggggcaaagttgctctgcccaccagaactgaagaggaggggctgcccactctgtaaatgaagggctcagtggagtctggccaagcacagaggctgctgtcaggaagagggaggaagaagcctgtgcagggggctggctggatgttctctttactgaaaagacagtggcaaggaaaagcaaaaaaaaaaaaaaaaaaaaaaaaaaa

SEQ ID NO: 2 - protein, 283 aa, Homo sapiens
DVAVDGQNWTFAFDFSFLSQQEDLAWAELRLQLSSPVDLPTEGSLAIEIFHQPKPDTEQASDSCLERFQMDLFTVTLSQVTFSLGSMVLEVTRPLSKWLKRPGALEKQMSRVAGECWPRPPTPPATNVLLMLYSNLSQEQRQLGGSTLLWEAESSWRAQEGQLSWEWGKRHRRHHLPDRSQLCRKVKFQVDFNLIGWGSWIIYPKQYNAYRCEGECPNPVGEEFHPTNHAYIQSLLKRYQPHRVPSTCCAPVKTKPLSMLYVDNGRVLLDHHKDMIVEECGCL

SEQ ID NO: 3 - DNA, 1688 bp, Homo sapiens
  CDS (53)..(1150)
gccttctcaagggacagccccactctgcctcttgctcctccagggcagcaccatgcagcccctgtggctctgctgggcactctgggtgttgcccctggccagccccggggccgccctgaccggggagcagctcctgggcagcctgctgcggcagctgcagctcaaagaggtgcccaccctggacagggccgacatggaggagctggtcatccccacccacgtgagggcccagtacgtggccctgctgcagcgcagccacggggaccgctcccgcggaaagaggttcagccagagcttccgagaggtggccggcaggttcctggcgttggaggccagcacacacctgctggtgttcggcatggagcagcggctgccgcccaacagcgagctggtgcaggccgtgctgcggctcttccaggagccggtccccaaggccgcgctgcacaggcacgggcggctgtccccgcgcagcgcccgggcccgggtgaccgtcgagtggctgcgcgtccgcgacgacggctccaaccgcacctccctcatcgactccaggctggtgtccgtccacgagagcggctggaaggccttcgacgtgaccgaggccgtgaacttctggcagcagctgagccggccccggcagccgctgctgctacaggtgtcggtgcagagggagcatctgggcccgctggcgtccggcgcccacaagctggtccgctttgcctcgcagggggcgccagccgggcttggggagccccagctggagctgcacaccctggaccttggggactatggagctcagggcgactgtgaccctgaagcaccaatgaccgagggcacccgctgctgccgccaggagatgtacattgacctgcaggggatgaagtgggccgagaactgggtgctggagcccccgggcttcctggcttatgagtgtgtgggcacctgccggcagcccccggaggccctggccttcaagtggccgtttctggggcctcgacagtgcatcgcctcggagactgactcgctgcccatgatcgtcagcatcaaggagggaggcaggaccaggccccaggtggtcagcctgcccaacatgagggtgcagaagtgcagctgtgcctcggatggtgcgctcgtgccaaggaggctccagccataggcgcctagtgtagccatcgagggacttgacttgtgtgtgtttctgaagtgttcgagggtaccaggagagctggcgatgactgaactgctgatggacaaatgctctgtgctctctatgagccctgaatttgcttcctctgacaagttacctcacctaatttttgcttctcaggaatgagaatctttggccactggagagcccttgctcagttttctctattcttattattcactgcactatattctaagcacttacatgtggagatactgtaacctgagggcagaaagcccaatgtgtcattgtttacttgtcctgtcactggatctgggctaaagtcctccaccaccactctggacctaagacctggggttaagtgtgggttgtgcatccccaatccagataataaagactttgtaaaacatgaataaaacacattttattctaaaaaaaaaaacggcacgagggggggcccggtacccaattcgccctatagtgagtcgtattacaattcactggccgtcgttttacaacgtcg

SEQ ID NO: 4 - protein, 366 aa, Homo sapiens
MQPLWLCWALWVLPLASPGAALTGEQLLGSLLRQLQLKEVPTLDRADMEELVIPTHVRAQYVALLQRSHGDRSRGKRFSQSFREVAGRFLALEASTHLLVFGMEQRLPPNSELVQAVLRLFQEPVPKAALHRHGRLSPRSARARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLGDYGAQGDCDPEAPMTEGTRCCRQEMYIDLQGMKWAENWVLEPPGFLAYECVGTCRQPPEALAFKWPFLGPRQCIASETDSLPMIVSIKEGGRTRPQVVSLPNMRVQKCSCASDGALVPRRLQP

SEQ ID NO: 5 - protein, 354 aa, Mus musculus
MSAHSLRILLLQACWALLHPRAPTAAALPLWTRGQPSSPSPLAYMLSLYRDPLPRADIIRSLQAQDVDVTGQNWTFTFDFSFLSQEEDLVWADVRLQLPGPMDIPTEGPLTIDIFHQAKGDPERDPADCLERIWMETFTVIPSQVTFASGSTVLEVTKPLSKWLKDPRALEKQVSSRAEKCWHQPYTPPVPVASTNVLMLYSNRPQEQRQLGGATLLWEAESSWRAQEGQLSVERGGWGRRQRRHHLPDRSQLCRRVKFQVDFNLIGWGSWIIYPKQYNAYRCEGECPNPVGEEFHPTNHAYIQSLLKRYQPHRVPSTCCAPVKTKPLSMLYVDNGRVLLEHHKDMIVEECGCL

SEQ ID NO: 6 - protein, 368 aa, Mus musculus
MPFLWLCWALWALSLVSLREALTGEQILGSLLQQLQLDQPPVLDKADVEGMVIPSHVRTQYVALLQHSHASRSRGKRFSQNLREVAGRFLVSETSTHLLVFGMEQRLPPNSELVQAVLRLFQEPVPRTALRRQKRLSPHSARARVTIEWLRFRDDGSNRTALIDSRLVSIHESGWKAFDVTEAVNFWQQLSRPRQPLLLQVSVQREHLGPGTWSSHKLVRFAAQGTPDGKGQGEPQLELHTLDLKDYGAQGNCDPEAPVTEGTRCCRQEMYLDLQGMKWAENWILEPPGFLTYECVGSCLQLPESLTSRWPFLGPRQCVASEMTSLPMIVSVKEGGRTRPQVVSLPNMRVQTCSCASDGALIPRRLQP

SEQ ID NO: 7 - DNA, 287 bp, Homo sapiens
ggcaagcagctcctgggcagcctgctggcactctacaagaggtgccaaacctggacagggcgacatggaggagctggtcatccccacccacgtagggaaccagtacgtggccctgctgcagcgccaacggggaaccactcccggaaaagaggttcagccagagcttccggcagcccccggagccctggccttcaagtggccgtttttggggcctcgacagtcatcgctcggagactgattcgtgcccatgatcgtcaacatcaaggagggaggcaggaccagcccca

SEQ ID NO: 8 - DNA, 104 bp, Homo sapiens
tcaaggggcagccccactctgcctcttgtccttccaggggtagcaccatgcagcccctgtggatctgctgggcactctgggtgttgcccctgggcacccggggc

SEQ ID NO: 9 - DNA, 29 bp, Artificial sequence
  Contains a BamHI restriction site
cgcggatcccatcacttgccagacagaag

SEQ ID NO: 10 - DNA, 42 bp, Artificial sequence
  Contains a HindIII restriction site
gtacgcaagcttgcaggcaaatccagtctccctccagggatg

SEQ ID NO: 11 - DNA, 36 bp, Artificial sequence
  Contains a BamHI restriction site
caattggatccacttgccagacagagaactcaactg

SEQ ID NO: 12 - DNA, 39 bp, Artificial sequence
  Contains an Asp718 restriction site
cacttaggtaccatgtcatcagaggcacccacattcttc

SEQ ID NO: 13 - DNA, 131 bp, Artificial sequence
  Contains a BamHI restriction site, a Kozak sequence, an AUG start codon, and a sequence encoding the secretory leader peptide from the human IL-6 gene
gccggatccgccaccatgaactccttctccacaagcgccttcggtccagttgccttctccctggggctgctcctggtgttgcctgctgccttccctgccccagtcatcacttgccagacagaagtcaactg

SEQ ID NO: 14 - DNA, 36 bp, Artificial sequence
  Contains an XbaI restriction site
ggctctagaatgtcatcagaggcacccacattcttc

SEQ ID NO: 15 - DNA, 36 bp, Artificial sequence
  Contains a BamHI restriction site, a Kozak sequence, and an AUG start codon
gactggatcccatacttgccagacagaagtcaactg

SEQ ID NO: 16 - DNA, 39 bp, Artificial sequence; identical to SEQ ID NO: 12
  Contains a BamHI restriction site

(19) United States
(12) Patent Application Publication
(10) Pub. No.: US 2003/0069408 A1
(43) Pub. Date: Apr. 10, 2003

(54) Human nodal and lefty homologues

(75) Inventors: Reinhard Ebner, Gaithersburg, MD (US); Daniel R. Soppet, Centreville, VA (US); Steven M. Ruben, Olney, MD (US)

Correspondence Address: HUMAN GENOME SCIENCES INC, 9410 KEY WEST AVENUE, ROCKVILLE, MD, 20850

(73) Assignee: Human Genome Sciences, Inc., Rockville, MD (US)

(21) Appl. No.: 10/166,183

(22) Filed: Jun. 11, 2002

Related U.S. Application Data
(63) Continuation of application No. 09/137,415, filed on Aug. 20, 1998.
(60) Provisional application No. 60/056,565, filed on Aug. 21, 1997.

(51) Int. Cl. C12P 21/02; C12N 5/06; C07K 14/495; C07H 21/04

(52) U.S. Cl. 536/23.5; 530/350; 435/69.1; 435/320.1; 435/325

(57) ABSTRACT

The present invention relates to novel Nodal and Lefty proteins which are members of the TGF-β family. In particular, isolated nucleic acid molecules are provided encoding the human Nodal and Lefty proteins. Nodal and Lefty polypeptides are also provided as are vectors, host cells and recombinant methods for producing the same. The invention further relates to screening methods for identifying agonists and antagonists of Nodal and Lefty activity. Also provided are diagnostic methods for detecting cell growth and differentiation-related disorders and therapeutic methods for treating cell growth and differentiation-related disorders.

### CROSS REFERENCE TO RELATED APPLICATIONS

This application is a continuation of and claims priority under 35 U.S.C. § 120 to U.S. application Ser. No. 09/137,415, filed Aug. 20, 1998, which is a nonprovisional of and claims benefit under 35 U.S.C. § 119(e) of U.S. Provisional Application No. 60/056,565, filed on Aug. 21, 1997, which is hereby incorporated by reference in its entirety.

### FIELD OF THE INVENTION

The present invention relates to two novel human genes encoding polypeptides which are members of the transforming growth factor-beta (TGF-β) superfamily. More specifically, isolated nucleic acid molecules are provided encoding human polypeptides designated the Nodal and Lefty homologues, hereinafter referred to as “Nodal” and “Lefty”, respectively. Nodal and Lefty polypeptides are also provided, as are vectors, host cells and recombinant methods for producing the same. Also provided are diagnostic methods for detecting disorders related to the regulation of cell growth and differentiation and therapeutic methods for treating such disorders. The invention further relates to screening methods for identifying agonists and antagonists of Nodal and Lefty activity.

### BACKGROUND OF THE INVENTION

The TGF-β family of peptide growth factors includes at least five members (TGF-β1 through TGF-β5) all of which form homodimers of approximately 25 kd. The TGF-β family belongs to a larger, extended super family of peptide signaling molecules that includes the Muellerian inhibiting substance (Cate, R. L., et al., Cell 45:685-698 (1986)), decapentaplegic (Padgett, R. W., et al., Nature 325:81-84 (1987)), bone morphogenic factors (Wozney, J. M., et al., Science 242:1528-1534 (1988)), vg1 (Weeks, D. L. and Melton, D. A., Cell 51:861-867 (1987)), activins (Vale, W., et al., Nature 321:776-779 (1986)), and inhibins (Mason, A. J., et al., Nature 318:659-663 (1985)). These factors are similar to TGF-β in overall structure, but share only approximately 25% amino acid identity with the TGF-β proteins and with each other. All of these molecules are thought to play an important roles in modulating growth, development and differentiation (Kingsley, D. M. Genes & Dev. 8:133-146 (1994)).

TGF-β was originally described as a factor that induced normal rat kidney fibroblasts to proliferate in soft agar in the presence of epidermal growth factor (Roberts, A. B., et al., Proc. Natl. Acad. Sci. USA 78:5339-5343 (1981)). TGF-β has subsequently been shown to exert a number of different effects in a variety of cells. For example, TGF-β can inhibit the differentiation of certain cells of mesodermal origin (Florini, J. R., et al., J. Biol. Chem. 261:1659-16513 (1986)), induced the differentiation of others (Seyedine, S. M. et al., Proc. Natl. Acad. Sci. USA 82:2267-2271 (1985)), and potently inhibit proliferation of various types of epithelial cells, (Tucker, R. F., Science 226:705-707 (1984)). This last activity has lead to the speculation that one important physiologic role for TGF-β is to maintain the repressed growth state of many types of cells. Accordingly, cells that lose the ability to respond to TGF-β are more likely to exhibit uncontrolled growth and to become tumorigenic. Indeed, cells which characteristically lack certain tumors (e.g. retinoblastoma) lack detectable TGF-β receptors at their cell surface and fail to respond to TGF-β, while their normal counterparts express self-surface receptors in their growth is potently inhibited by TGF-β (Kim Chi, A., et al., Science 240:196-198 (1988)).

More specifically, TGF-β1 stimulates the anchorage-independent growth of normal rat kidney fibroblasts (Robert et al., Proc. Natl. Acad. Sci. USA 78:5339-5343 (1981)). Since then it has been shown to be a multi-functional regulator of cell growth and differentiation (Spom, et al., Science 233:532-534 (1986)) being capable of such diverse effects of inhibiting the growth of several human cancer cell lines (Roberts, et al., Proc. Natl. Acad. Sci. USA 82:119-123 (1985)), mouse keratinocytes, (Coffey, et al., Cancer Res. 48:1596-1602 (1988)), and T and B lymphocytes (Kehrl, et al., J. Exp. Med. 163:1037-1050 (1986)). It also inhibits early hematopoietic progenitor cell proliferation (Goey, et al., J. Immunol. 143:877-880 (1989)), stimulates the induction of differentiation of rat muscle mesenchymal cells and subsequent production of cartilage-specific macro molecules (Seyedine, et al., J. Biol. Chem. 262:1946-1949 (1986)), causes increased synthesis and secretion of collagen (Ignotz, et al., J. Biol. Chem. 261:4337-4345 (1986)), stimulates bone formation (Noda, et al., Endocrinol. 124:2991-2995 (1989)), and accelerates the healing of incision wounds (Mustoe, et al., Science 237:1333-1335 (1987)).

Further, TGF-β1 stimulates formation of extracellular matrix molecules in the liver and lung. When levels of TGF-β1 are higher than normal, formation of fiber occurs in the extracellular matrix of the liver and lung which can be fatal. High levels of TGF-β1 occur due to chemotherapy and bone marrow transplant as an attempt to treat cancers such as breast cancer.

A second protein termed TGF-β2 was isolated from several sources including demineralized bone, a human prostatic adenocarcinoma cell line (Ikeda, et al., J. Bio. Chem. 26:2406-2410 (1987)). TGF-β2 shared several functional similarities with TGF-β1. These proteins are now known to be members of a family of related growth modulatory proteins including TGF-β3 (Ten-Dijke, et al., Proc. Natl. Acad. Sci. USA 85:471-4719 (1988)), Muellerian inhibitory substance and the inhibins.

Thus, there is a need for polypeptides that function as potent regulators of cell growth and differentiation, since disturbances of such regulation may be involved in disorders relating to abnormal regulation of cell growth and differentiation, cancer, tissue regeneration, and wound healing. Therefore, there is a need for identification and characterization of such human polypeptides which can play a role in detecting, preventing, ameliorating or correcting such disorders.

### SUMMARY OF THE INVENTION

The present invention provides isolated nucleic acid molecules comprising polynucleotides encoding at least a portion of the Nodal polypeptide having the complete amino acid sequence shown in SEQ ID NO:2 or the complete amino acid sequence encoded by the cDNA clone deposited as plasmid DNA as ATCC Deposit Number 209092, on Jun. 5, 1997 or the complete amino acid sequence encoded by the cDNA clone deposited as plasmid DNA as ATCC Deposit Number 209135, on Jul. 2, 1997. The nucleotide sequence determined by sequencing the deposited Nodal clone, which is shown in FIGS. 1A and B (SEQ ID NO:1), and contains a single open reading frame encoding a complete polypeptide of 283 amino acid residues initiating with a codon encoding an N-terminal aspartic acid residue at nucleotide positions 1-3 with a predicted molecular weight of about 32.5 kDa. Nucleic acid molecules of the invention include those encoding the complete amino acid sequence shown in SEQ ID NO:2, the complete amino acid sequence encoded by the cDNA clone in ATCC Deposit Numbers 209092 and 209135, which molecules also can encode additional amino acids fused to the N-terminus of the Nodal amino acid sequence.

The present invention also provides isolated nucleic acid molecules comprising polynueleotides encoding at least a portion of the Lefty polypeptide having the complete amino acid sequence shown in SEQ ID NO:4 or the complete amino acid sequence encoded by the cDNA clone deposited as plasmid DNA as ATCC Deposit Number 209091 on Jun. 5, 1997. The nucleotide sequences determined by sequencing the deposited Lefty clone, which is shown in FIGS. 2A and B (SEQ ID NO:3), and contains a single open reading frame encoding a complete polypeptide of 366 amino acid residues with an initiation codon encoding an N-terminal methionine at nucleotide positions 53-55, and a predicted molecular weight of about 40.9 kDa. Nucleic acid molecules of the invention include those encoding the complete amino acid sequence shown in SEQ ID NO:4, those encoding the complete amino acid sequence shown in SEQ ID NO:4 excluding the N-terminal methionine, the complete amino acid sequences encoded by the cDNA clone in ATCC Deposit Numbers 209091, or the complete amino acid sequences excepting the N-terminal methionine encoded by the cDNA clone in ATCC Deposit Number 209091, which molecules also can encode additional amino acids fused to the N-terminus of the Lefty amino acid sequence.

The Nodal protein of the present invention shares sequence homology with the translation product of the murine mRNA for Nodal (FIG. 3; SEQ ID NO:5), including the conserved predicted active domain of about 110 amino acids. Murine Nodal is thought to be essential for mesoderm formation and subsequent organization of axial structures in early mouse development. The homology between murine Nodal and the human Nodal homologue of the present invention indicates that the human Nodal homologue of the present invention may also be involved in a developmental process such as the correct formation of various structures or in one or more post-developmental capacities including sexual development, pituitary hormone production, and the creation of bone and cartilage, as are many of the other members of the TGF-β superfamily.

The Lefty protein of the present invention shares sequence homology with the translation product of the murine mRNA for Lefty (FIG. 4; SEQ ID NO:6), including the conserved predicted active domain of about 110 amino acids. Murine Lefty is thought to be important in left/right handedness of the developing organism. The homology between murine Lefty and the novel human Lefty homologue of the present invention indicates that the novel human Lefty homologue of the present invention may also be involved in correct formation of various structures with respect to the rest of the developing organism or Lefty may also be involved in one or more post-developmental capacities including sexual development, pituitary hormone production, and the creation of bone and cartilage, as are many of the other members of the TGF-β superfamily.

Nodal and Lefty polypeptides of the present invention are useful for enhancing or enriching the growth and/or differentiation of specific cell populations, e.g., embryonic cells or stem cells.

Another embodiment of the invention provides pharmaceutical compositions which contain a therapeutically effective amount of human Nodal and/or Lefty polypeptide, in a pharmaceutically acceptable vehicle or carrier. These compositions of the invention may be useful in the therapeutic modulation or diagnosis of bone, cartilage, or other connective cell or tissue growth and/or differentiation. These compositions may be used to treat such conditions as osteoarthritis, osteoporosis, and other abnormalities of bone, cartilage, muscle, tendon, ligament and/or other connective tissues and/or organs such as liver, lung, cardiac, pancreas, kidney, and other tissues. These compositions may also be useful in the growth and/or formation of cartilage, tendon, ligament, meniscus, and other connective tissues or any combination of the above (e.g., therapeutic modulation of the tendon-to-bone attachment apparatus). These compositions may also be useful in treating periodontal disease and modulating wound healing and tissue repair of such tissues as epidermis, nerve, muscle, cardiac muscle, liver, lung, cardiac, pancreas, kidney, and other tissues and/or organs. Pharmaceutical compositions containing Nodal and/or Lefty of the invention may include one or more other therapeutically useful component such as BMP-1, BMP-2, BMP-3, BMP-4, BMP-5, BMP-6, and/or BMP-7 (See, for example, U.S. Pat. Nos. 5,108,922; 5,013,649; 5,116,738; 5,106,748; 5,187,076; and 5,141,905), BMP-8 (See, for example, PCT publication WO91/18098), BMP-9 (See, for example, PCT publication WO93/00432), BMP-10 (See, for example, PCT publication WO94/26893), BMP-11 (See, for example, PCT publication WO94/26892), BMP-12 and/or BMP-13 (See, for example, PCT publication WO95/16035), with other growth factors including, but not limited to, BIP, one or more of the growth and differentiation factors (GDFs), VGR-2, epidermal growth factor (EGF), fibroblast growth factor (FGF), TGF-alpha, TGF-beta, activins, inhibins, and insulin-like growth factor (IGF).

The encoded Lefty polypeptide has a predicted leader sequence of 18 amino acids underlined in FIG. 2A; and the amino acid sequence of the predicted secreted Lefty protein is also shown in FIGS. 2A-B, as amino acid residues 19-366 and as residues 1-348 in SEQ ID NO:4.

Thus, one embodiment of the invention provides an isolated nucleic acid molecule comprising a polynucleotide having a nucleotide sequence selected from the group consisting of: (a) a nucleotide sequence encoding the Nodal polypeptide having the complete amino acid sequence in SEQ ID NO:2 (i.e., positions 1 to 283 of SEQ ID NO:2); (b) a nucleotide sequence encoding the predicted active Nodal polypeptide having the amino acid sequence at positions 173 to 283 of SEQ ID NO:2; (c) a nucleotide sequence encoding the Nodal polypeptide having the complete amino acid sequence encoded by the cDNA clone contained in ATCC Deposit No. 209092 and/or 209135; (d) a nucleotide sequence encoding the active domain of the Nodal polypeptide having the amino acid sequence encoded by the cDNA clone contained in ATCC Deposit No. 209092 and/or 209135; and (e) a nucleotide sequence complementary to any of the nucleotide sequences in (a), (b), (c) or (d) above.

Another embodiment of the invention provides an isolated nucleic acid molecule comprising a polynucleotide having a nucleotide sequence selected from the group consisting of: (a) a nucleotide sequence encoding the Lefty polypeptide having the complete amino acid sequence in SEQ ID NO:4 (i.e., positions-18 to 348 of SEQ ID NO:4); (b) a nucleotide sequence encoding the Lefty polypeptide having the complete amino acid sequence in SEQ ID NO:4 excepting the N-terminal methionine (i.e., positions −17 to 348 of SEQ ID NO:4); (c) a nucleotide sequence encoding the predicted active domain of the Lefty polypeptide having the amino acid sequence at positions 60 to 348 of SEQ ID NO:4; (d) a nucleotide sequence encoding the predicted active domain of the Lefty polypeptide having the amino acid sequence at positions 118 to 348 of SEQ ID NO:4; (e) a nucleotide sequence encoding the predicted active domain of the Lefty polypeptide having the amino acid sequence at positions 125 to 348 of SEQ ID NO:4; (f) a nucleotide sequence encoding the Lefty polypeptide having the complete amino acid sequence encoded by the cDNA clone contained in ATCC Deposit No. 209091; (g) a nucleotide sequence encoding the Lefty polypeptide having the complete amino acid sequence excepting the N-terminal methionine encoded by the cDNA clone contained in ATCC Deposit No. 209091; (h) a nucleotide sequence encoding the active domain of the Lefty polypeptide having the amino acid sequence encoded by the cDNA clone contained in ATCC Deposit No. 209091; and (i) a nucleotide sequence complementary to any of the nucleotide sequences in (a), (b), (c), (d), (e), (f), (g) or (h) above.

Further embodiments of the invention include isolated nucleic acid molecules that comprise a polynucleotide having a nucleotide sequence at least 90% identical, and more preferably at least 95%, 96%, 97%, 98% or 99% identical, to any of the nucleotide sequences in (a), (b), (c), (d) or (e), above, with regard to Nodal, to any of the nucleotide sequences in (a), (b), (c), (d), (e), (f), (g), (h) or (i), above, with regard to Lefty, or a polynucleotide which hybridizes, preferably under stringent hybridization conditions, to a polynucleotide in (a), (b), (c), (d) or (e), above, with regard to Nodal, or any of the nucleotide sequences in (a), (b), (c), (d), (e), (f), (g), (h) or (i), above, with regard to Lefty, listed above. This polynucleotide which hybridizes does not hybridize under stringent hybridization conditions to a polynucleotide having a nucleotide sequence consisting of only A residues or of only T residues.

An additional nucleic acid embodiment of the invention relates to an isolated nucleic acid molecule comprising a polynucleotide which encodes the amino acid sequence of an epitope-bearing portion of a Nodal polypeptide having an amino acid sequence in (a), (b), (c), (d) or (e), with regard to Nodal, above. A further nucleic acid embodiment of the invention relates to an isolated nucleic acid molecule comprising a polynucleotide which encodes the amino acid sequence of an epitope-bearing portion of a Lefty polypeptide having an amino acid sequence in (a), (b), (c), (d), (e), (f), (g), (h) or (i), with regard to Lefty, above. A further embodiment of the invention relates to an isolated nucleic acid molecule comprising a polynucleotide which encodes the amino acid sequences of a Nodal or Lefty polypeptide having an amino acid sequence which contains at least one amino acid substitution, but not more than 50 amino acid substitutions, even more preferably, not more than 40 amino acid substitutions, still more preferably, not more than 30 amino acid substitutions, and still even more preferably, not more than 20 amino acid substitutions. Of course, in order of ever-increasing preference, it is highly preferable for a polynucleotide which encodes the amino acid sequence of a Nodal or Lefty polypeptide to have an amino acid sequence which contains not more than 10, 9, 8, 7, 6, 5, 4, 3, 2 or 1 amino acid substitutions. Conservative substitutions are preferable.

The present invention also relates to recombinant vectors, which include the isolated nucleic acid molecules of the present invention, and to host cells containing the recombinant vectors, as well as to methods of making such vectors and host cells and for using them for production of Nodal or Lefty polypeptides or peptides by recombinant techniques.

In accordance with a further embodiment of the present invention, there is provided a process for producing such polypeptide by recombinant techniques comprising culturing recombinant prokaryotic and/or eukaryotic host cells, containing a human Nodal or Lefty nucleic acid sequence, under conditions promoting expression of said protein and subsequent recovery of said protein.

The invention further provides an isolated Nodal or Lefty polypeptide comprising an amino acid sequence selected from the group consisting of: (a) the amino acid sequence of the full-length Nodal polypeptide having the complete amino acid sequence shown in SEQ ID NO:2 (i.e., positions 1 to 283 of SEQ ID NO:2); (b) the amino acid sequence of the predicted active Nodal polypeptide having the amino acid sequence at positions 173 to 283 of SEQ ID NO:2; (c) the amino acid sequence of the Nodal polypeptide having the complete amino acid sequence encoded by the cDNA clone contained in ATCC Deposit No. 209092 and/or 209135; (d) the amino acid sequence of the active domain of the Nodal polypeptide having the amino acid sequence encoded by the cDNA clone contained in ATCC Deposit No. 209092 and/or 209135; (e) the amino acid sequence of the Lefty polypeptide having the complete amino acid sequence in SEQ ID NO:4 (i.e., positions −18 to 348 of SEQ ID NO:4); (f) the amino acid sequence of the Lefty polypeptide having the complete amino acid sequence in SEQ ID NO:4 excepting the N-terminal methionine (i.e., positions −17 to 348 of SEQ ID NO:4); (g) the amino acid sequence of the predicted active domain of the Lefty polypeptide having the amino acid sequence at positions 60 to 348 of SEQ ID NO:4; (h) the amino acid sequence of the predicted active domain of the Lefty polypeptide having the amino acid sequence at positions 118 to 348 of SEQ ID NO:4; (i) the amino acid sequence of the predicted active domain of the Lefty polypeptide having the amino acid sequence at positions 125 to 348 of SEQ ID NO:4; 0) the amino acid sequence of the Lefty polypeptide having the complete amino acid sequence encoded by the cDNA clone contained in ATCC Deposit No. 209091; (k) the amino acid sequence of the Lefty polypeptide having the complete amino acid sequence excepting the N-terminal methionine encoded by the cDNA clone contained in ATCC Deposit No. 209091, and; (1) the amino acid sequence of the active domain of the Lefty polypeptide having the amino acid sequence encoded by the cDNA clone contained in ATCC Deposit No. 209091. The polypeptides of the present invention also include polypeptides having an amino acid sequence at least 80% identical, more preferably at least 90% identical, and still more preferably 95%, 96%, 97%, 98% or 99% identical to those described in (a) through (l) above, as well as polypeptides having an amino acid sequence with at least 90% similarity, and more preferably at least 95% similarity, to those above.

An additional embodiment of the invention relates to a peptide or polypeptide which comprises the amino acid sequence of an epitope-bearing portion of a Nodal or Lefty polypeptide having an amino acid sequence described in (a) through (l), above, Peptides or polypeptides having the amino acid sequence of an epitope-bearing portion of a Nodal or Lefty polypeptide of the invention include portions of such polypeptides with at least six or seven, preferably at least nine, and more preferably at least about 30 amino acids to about 50 amino acids, although epitope-bearing polypeptides of any length up to and including the entire amino acid sequence of a polypeptide of the invention described above also are included in the invention.

A further embodiment of the invention relates to a polypeptide which comprises the amino acid sequence of a Nodal or Lefty polypeptide having an amino acid sequence which contains at least one amino acid substitution, but not more than 50 amino acid substitutions, even more preferably, not more than 40 amino acid substitutions, still more preferably, not more than 30 amino acid substitutions, and still even more preferably, not more than 20 amino acid substitutions. Of course, in order of ever-increasing preference, it is highly preferable for a peptide or polypeptide to have an amino acid sequence which comprises the amino acid sequence of a TNF-gamma polypeptide, which contains at least one, but not more than 10, 9, 8, 7, 6, 5, 4, 3, 2 or 1 amino acid substitutions. In specific embodiments, the number of additions, substitutions, and/or deletions in the amino acid sequence of FIGS. 1A and 1B, FIGS. 2A and 2B, or fragments thereof (e.g., the mature form and/or other fragments described herein), is 1-5, 5-10, 5-25, 5-50, 10-50 or 50-150, conservative amino acid substitutions are preferable.

In another embodiment, the invention provides an isolated antibody that binds specifically to a Nodal and Lefty polypeptide having an amino acid sequence described in (a) through (l) above. The invention further provides methods for isolating antibodies that bind specifically to a Nodal or Lefty polypeptide having an amino acid sequence as described herein. Such antibodies are useful diagnostically or therapeutically as described below.

The invention also provides for pharmaceutical compositions comprising Nodal and Lefty polypeptides, particularly human Nodal and Lefty polypeptides, which may be employed, for instance, to treat cellular growth and differentiation disorders. Methods of treating individuals in need of Nodal and Lefty polypeptides are also provided.

The invention farther provides compositions comprising a Nodal or Lefty polynucleotide or a Nodal or Lefty polypeptide for administration to cells in vitro, to cells ex vivo and to cells in vivo, or to a multicellular organism. In certain particularly preferred embodiments of the invention, the compositions comprise a Nodal or Lefty polynucleotide for expression of a Nodal or Lefty polypeptide in a host organism for treatment of disease. Particularly preferred in this regard is expression in a human patient for treatment of a dysfunction associated with aberrant endogenous activity of Nodal or Lefty.

The present invention also provides a screening method for identifying compounds capable of enhancing or inhibiting a biological activities of the Nodal and Lefty polypeptides, which involves contacting a receptor which is enhanced by the Nodal or Lefty polypeptides with the candidate compound in the presence of a Nodal or Lefty polypeptide, assaying receptor activation in the presence of the candidate compound and of Nodal or Lefty polypeptide, and comparing the receptor activity to a standard level of activity, the standard being assayed when contact is made between the receptor and in the presence of the Nodal or Lefty polypeptide and the absence of the candidate compound In this assay, an increase in receptor activation over the standard indicates that the candidate compound is an agonist of Nodal or Lefty activity and a decrease in receptor activation compared to the standard indicates that the compound is an antagonist of Nodal or Lefty activity.

In another embodiment, a screening assay for agonists and antagonists is provided which involves determining the effect a candidate compound has on Nodal or Lefty binding to a receptor. In particular, the method involves contacting the receptor with a Nodal or Lefty polypeptide and a candidate compound and determining whether Nodal or Lefty polypeptide binding to the receptor is increased or decreased due to the presence of the candidate compound. In this assay, an increase in binding of Nodal or Lefty over the standard binding indicates that the candidate compound is an agonist of Nodal or Lefty binding activity and a decrease in Nodal or Lefty binding compared to the standard indicates that the compound is an antagonist of Nodal or Lefty binding activity.

It has been discovered that, by detection in the HGS EST database, Nodal is expressed not only in neutrophils, but also in testes. In addition, it has been discovered that, by detection in the HGS EST database, Lefty is expressed not only in uterine cancer, but also in colon cancer, apoptotic T-cells, fetal heart, Wilm's Tumor tissue, frontal lobe of the brain from a patient with dementia, neutrophils, salivary gland, small intestine, 7, 8, and 12 week old human embryos, frontal cortex and hypothalamus from a patient with schizophrenia, brain from a patient with Alzheimer's Disease, adipose tissue, brown fat, TNF- and LPS-induced and uninduced bone marrow stroma, activated monocytes and macrophages, rhabdomyosarcoma, cycloheximide-treated Raji cells, breast lymph nodes, hemangiopericytoma, testes, fetal epithelium (skin), and IL-5-induced eosinophils. Therefore, nucleic acids of the invention are useful as hybridization probes for differential identification of the tissue(s) or cell type(s) present in a biological sample. Similarly, polypeptides and antibodies directed to those polypeptides are useful to provide immunological probes for differential identification of the tissue(s) or cell type(s). In addition, for a number of disorders of the above tissues or cells, particularly with regard to the regulation of cell growth and differentiation, significantly higher or lower levels of Nodal or Lefty gene expression may be detected in certain tissues (e.g., cancerous and wounded tissues) or bodily fluids (e.g., serum, plasma, urine, synovial fluid or spinal fluid) taken from an individual having such a disorder, relative to a “standard” Nodal or Lefty gene expression level, i.e., the Nodal and Lefty expression levels in healthy tissue from an individual not having the cell growth and differentiation disorder. Thus, the invention provides a diagnostic method useful during diagnosis of such a disorder, which involves: (a) assaying Nodal and Lefty gene expression level in cells or body fluid of an individual; (b) comparing the Nodal and Lefty gene expression levels with standard Nodal and Lefty gene expression levels, whereby an increase or decrease in the assayed Nodal and Lefty gene expression level compared to the standard expression level is indicative of disorder in the regulation of cell growth and differentiation.

An additional embodiment of the invention is related to a method for treating an individual in need of an increased level of Nodal or Lefty activity in the body comprising administering to such an individual a composition comprising a therapeutically effective amount of an isolated Nodal or Lefty polypeptide of the invention or an agonist thereof.

A still further embodiment of the invention is related to a method for treating an individual in need of a decreased level of Nodal or Lefty activity in the body comprising, administering to such an individual a composition comprising a therapeutically effective amount of a Nodal or Lefty antagonist. Preferred antagonists for use in the present invention are Nodal- or Lefty-specific antibodies.

### BRIEF DESCRIPTION OF THE FIGURES

FIGS. 1A and 1B show the nucleotide sequence (SEQ ID NO:1) and deduced amino acid sequence (SEQ ID NO:2) of the human Nodal homologue of the present invention.

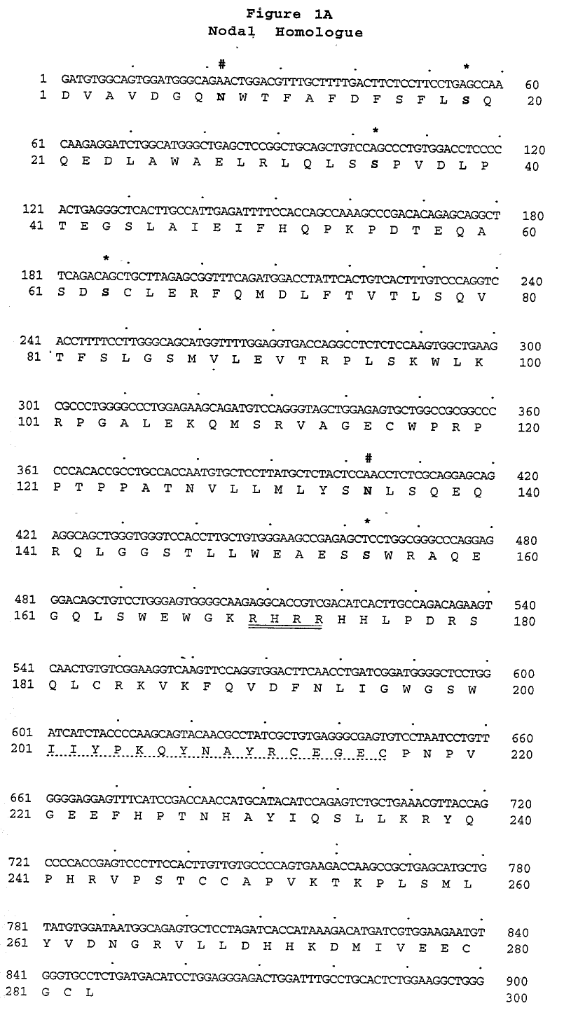

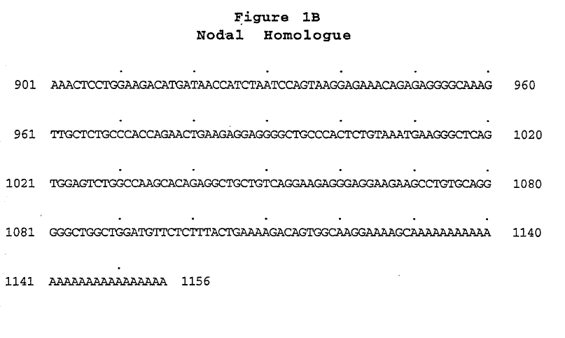

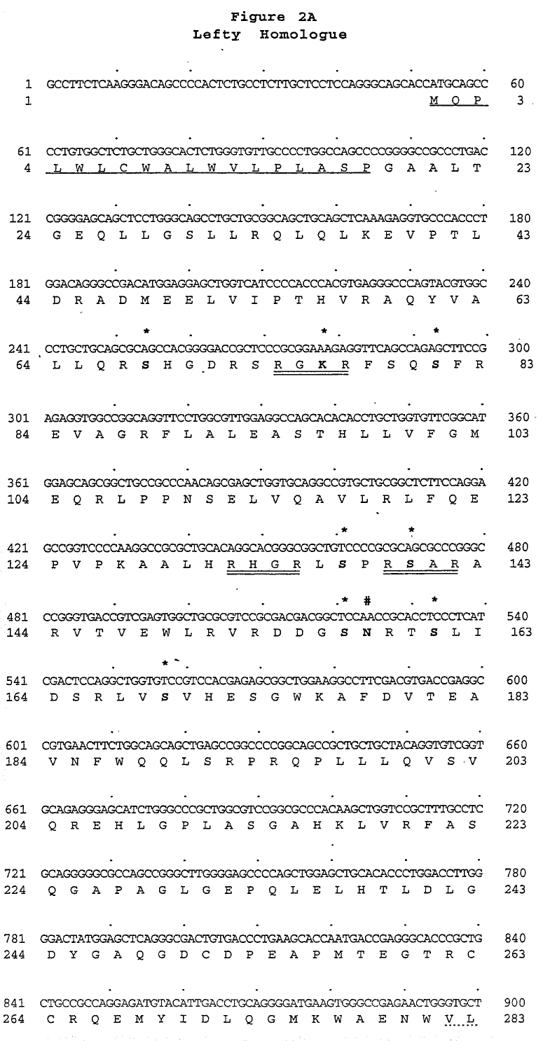

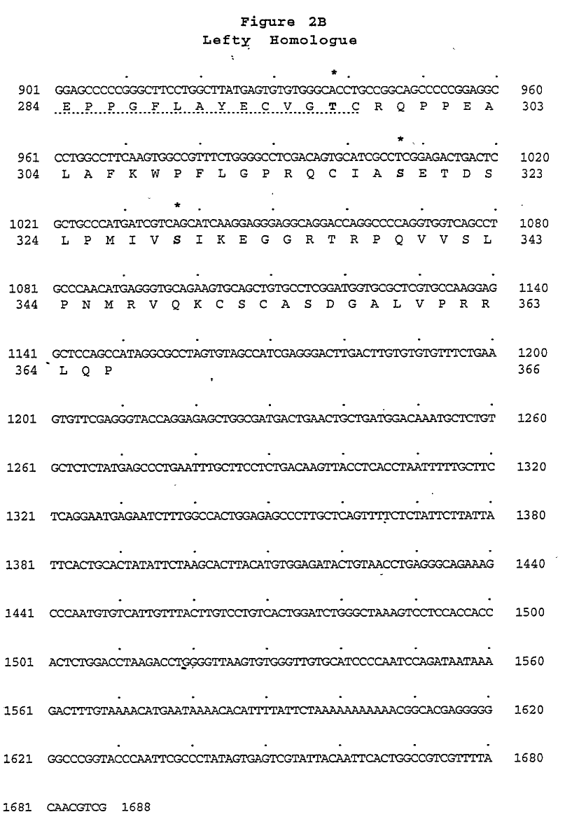

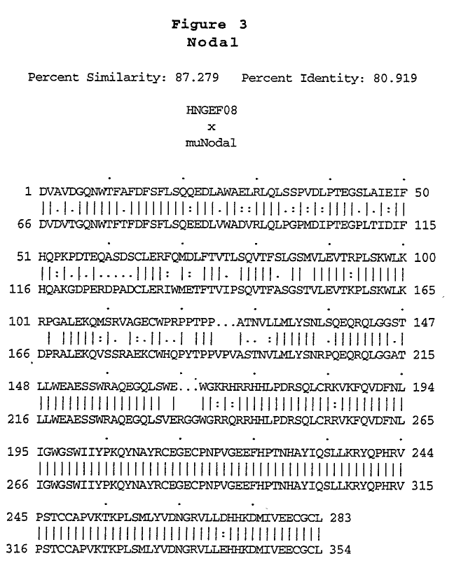

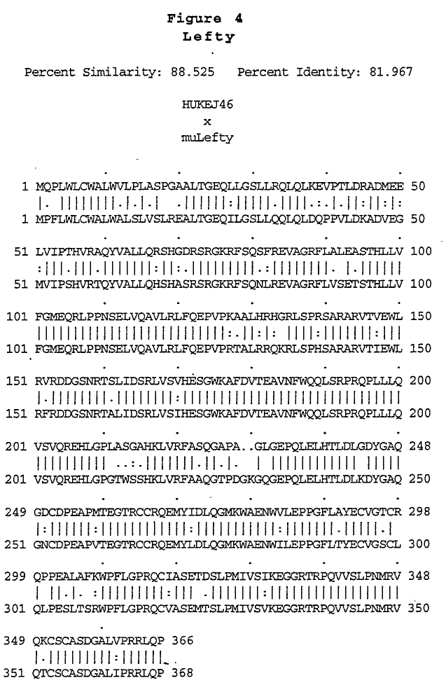

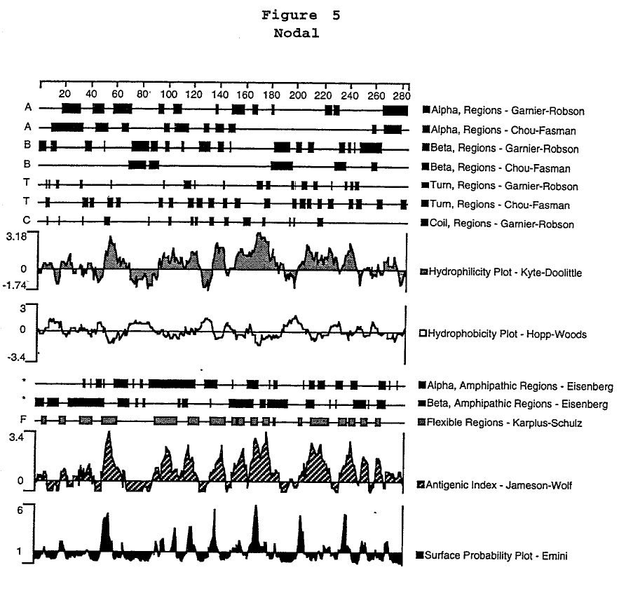

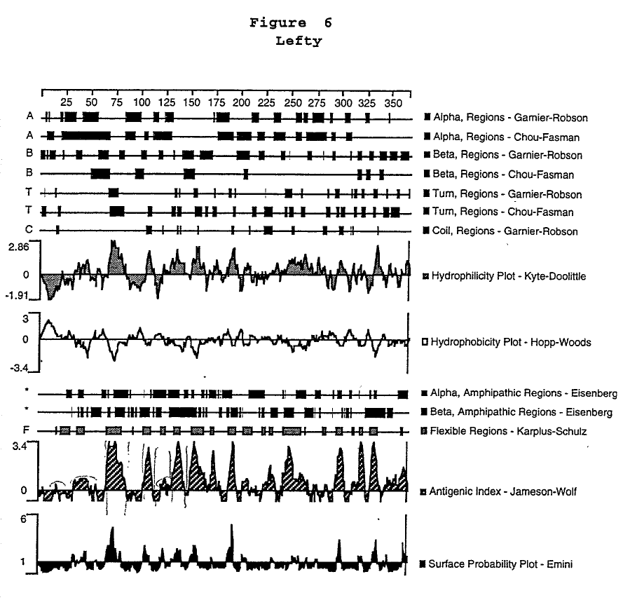

The predicted TGF-β consensus cleavage sequences (arginine-X-X-arginine (RXXR); where X is any amino acid) of the human Nodal homologue is double underlined in FIGS. 1A and 1B. The TGF-β consensus cleavage sequence appears once in the amino acid sequence of Nodal. Cleavage of the precursor form of human Nodal is predicted to occur immediately after the C-terminal arginine in the abovementioned consensus sequence in the amino acid sequence of Nodal.

Potential asparagine-linked glycosylation sites are marked in FIGS. 1A and 1B with a bolded asparagine symbol (N) in the Nodal amino acid sequence and a bolded pound sign (#) above the first nucleotide encoding that asparagine residue in the Nodal nucleotide sequence. Potential N-linked glycosylation sequences are found at the following locations in the Nodal amino acid sequence: N-8 through F-11 (N-8, W-9, T-10, F-11) and N-135 through Q-138 (N-135, L-136, S-137, Q-138). A potential Protein Kinase C (PKC) phosphorylation site is also marked in FIGS. 1A and 1B with a bolded serine symbol (S) in the Nodal amino acid sequence and an asterisk (*) above the first nucleotide encoding that serine residue in the Nodal nucleotide sequence. The potential PKC phosphorylation sequence is found in the Nodal amino acid sequence from residue S-155 through residue R-157 (S-155, W-156, R-157). Potential Casein Kinase II (CK2) phosphorylation sites are also marked in FIGS. 1A and 1B with a bolded serine symbol (S) in the Nodal amino acid sequence and an asterisk (*) above the first nucleotide encoding the appropriate serine residue in the Nodal nucleotide sequence. Potential CK2 phosphorylation sequences are found at the following locations in the Nodal amino acid sequence: S-19 through E-22 (S-19, Q-20, Q-21, E-22); S-35 through D-38 (S-35, P-36, V-37, D-38); and S-63 through E-66 (S-63, C-64, L-65, E-66). A potential myristylation site is found in the Nodal amino acid sequence in FIGS. 1A and 1B from residue G-6 through F-11 (G-6, Q-7, N-8, W-9, T-10, F-11). A potential amidation site is found in the Nodal amino acid sequence in FIGS. 1A and 1B from residue W-167 through R-170 (W-167, G-168, K-169, R-170). A TGF-beta family signature is found in the Nodal amino acid sequence in FIGS. 1A and 1B from residue I-201 through C-216 (I-201, I-202, Y-203, P-204, K-205, Q-206, Y-207, N-208, A-209, Y-210, R-21 1, C-212, E-213, G-214, E-215, C-216). This sequence is denoted in FIGS. 1A and 1B with a dotted underline shown under the amino acid sequence from residue I-201 through C-216.

FIGS. 2A and 2B show the nucleotide sequence (SEQ ID NO:3) and deduced amino acid sequence (SEQ ID NO:4) of the Lefty homologue of the present invention.

The predicted leader cleavage sequence of the human Lefty homologue of about 18 amino acids is underlined in FIG. 2A. Note that the methionine residue at the beginning of the leader sequence in FIG. 2A is shown in position number (positive or “+”) 1, whereas the leader positions in the corresponding sequence of SEQ ID NO:2 are designated with negative position numbers. Thus, the leader sequence positions 1 to 18 in FIG. 2A correspond to positions −18 to −1 in SEQ ID NO:2.

The predicted consensus sequences (arginine-X-X-arginine (RXXR); where X is any amino acid) of the human Lefty homologue is double underlined in FIGS. 2A and 2B. The TGF-β consensus cleavage sequence appears three times in the amino acid sequence of Lefty. Cleavage of the precursor forms of human Lefty is predicted to occur immediately after the C-terminal arginine in the abovementioned consensus sequence in the amino acid sequence of Lefty.

A potential asparagine-linked glycosylation site is marked in FIGS. 2A and 2B with a bolded asparagine symbol (N) in the Nodal amino acid sequence and a bolded pound sign (#) above the first nucleotide encoding that asparagine residue in the Lefty nucleotide sequence. The potential N-linked glycosylation sequence is found in the Lefty amino acid sequence from residue N-158 through S-161 (N-158, R-159, T-160, S-161). A potential cAMP- and cGMP-dependent protein kinase (CPK) phosphorylation site is marked in FIGS. 2A and 2B with a bolded serine symbol (S) in the Lefty amino acid sequence and an asterisk (*) above the first nucleotide encoding that serine residue in the Lefty nucleotide sequence. The potential CPK phosphorylation sequence is found in the Lefty amino acid sequence from residue K-76 through residue S-79 (K-76, R-77, F-78, S-79). Several potential Protein Kinase C (PKC) phosphorylation sites are also marked in FIGS. 2A and 2B with a bolded serine or threonine symbol (S or T) in the Lefty amino acid sequence and an asterisk (*) above the first nucleotide encoding that serine or threonine residue in the Lefty nucleotide sequence. The potential PKC phosphorylation sequences are found in the Lefty amino acid sequence from residue S-81 through residue R-83 (S-81, F-82, R-83); S-137 through R-139 (S-137, P-138, R-139); S-140 through R-142 (S-140, A-141, R-142); S-157 through R-159 (S-157, N-158, R-159); T-296 through R-298 (T-296, C-297, R-298); and S-329 through K-331 (S-329, I-330, K-331). Potential Casein Kinase II (CK2) phosphorylation sites are also marked in FIGS. 2A and 2B with a bolded serine symbol (S) in the Nodal amino acid sequence and an asterisk (*) above the first nucleotide encoding the appropriate serine residue in the Lefty nucleotide sequence. Potential CK2 phosphorylation sequences are found at the following locations in the Lefty amino acid sequence: S-68 through D-71 (S-68, H-69, G-70, D-71); S-81 through E-84 (S-81, F-82, R-83, E-84); S-161 through D-164 (S-161, L-162, I-163, D-164); S-169 through E-172 (S-169, V-170, H-171, E-172); S-319 through D-322 (S-319, E-320, T-321, D-322); and S-329 through E-332 (S-329, I-330, K-331, E-332). Several potential myristylation sites are found in the Lefty amino acid sequence in FIGS. 2A and 2B at the following locations: from residue G-19 through G-24 (G-19, A-20, A-21, L-22, T-23, G-24); G-156 through S-161 (G-156, S-157, N-158, R-159, T-160, S-161); G-225 through L-230 (G-225, A-226, P-227, A-228, G-229, L-230); G-260 through G-265 (G-260, T-261, R-262, C-263, C-264, R-265); and G-274 through G-279 (G-274, M-275, K-276, W-277, A-278, E-279). A potential amidation site is found in the Lefty amino acid sequence in FIGS. 2A and 2B from residue R-74 through R-77 (R-74, G-75, K-76, R-77). A TGF-beta family signature is found in the Lefty amino acid sequence in FIGS. 2A and 2B from residue V-282 through C-297 (V-282, L-283, E-284, P-285, P-286, G-287, F-288, L-289, A-290, Y-291, E-292, C-293, V-294, G-295, T-296, C-297). This sequence is denoted in FIGS. 2A and 2B with a dotted underline shown under the amino acid sequence from residue 1-282 through C-297.

FIGS. 3 and 4 show the regions of identity between the amino acid sequences of the Nodal and Lefty proteins (SEQ ID NO:2 and SEQ ID NO:4, respectively) and translation product of the murine mRNAs for Nodal and Lefty, respectively, (SEQ ID NO:5 and SEQ ID NO:6, respectively), determined by the computer program Bestfit (Wisconsin Sequence Analysis Package, Version 8 for Unix, Genetics Computer Group, University Research Park, 575 Science Drive, Madison, Wis. 53711) using the default parameters.

FIGS. 5 and 6 show computer analyses of the Nodal and Lefty amino acid sequences depicted in FIGS. 1A and 1B (SEQ ID NO:2) and 2A and 2B (SEQ ID NO:4), respectively. Alpha, beta, turn and coil regions; hydrophilicity and hydrophobicity; amphipathic regions; flexible regions; antigenic index and surface probability, as predicted using the default parameters of the recited programs, are shown. In the “Antigenic Index or Jameson-Wolf” graph, the positive peaks indicate locations of the highly antigenic regions of the Nodal and Lefty proteins, i.e., regions from which epitope-bearing peptides of the invention can be obtained. Non-limiting examples of antigenic polypeptides or peptides that can be used to generate Nodal-specific antibodies include: a polypeptide comprising amino acid residues from about Lys-54 to about Asp-62, from about Val-91 to about Leu-99, from about Lys-100 to about Gln-108, from about Cys-116 to about Pro-124, from about Gln-140 to about Leu-148, from about Trp-156 to about Ser-164, from about Arg-170, to about Gln-181, from about Cys-212 to about Phe-224, from about Tyr-239, to about Thr-247, from about Pro-251, to about Met-259, and from about Asp-263, to about His-271. Non-limiting examples of antigenic polypeptides or peptides that can be used to generate Lefty-specific antibodies include: a polypeptide comprising amino acid residues from about Asp-71 to about Ser-79, from about Arg-106 to about Val-114, from about Leu-136 to about Arg-144, from about Asp-154 to about Asp-164, from about His-171 to about Asp-179, from about Gln-189 to about Leu-197, from about Pro-227 to about Glu-236, from about Gly-246 to about Glu-254, from about Pro-256 to about Gln-266, from about Cys-297 to about Ala-305, from about Ile-317 to about Pro-325, from about Ile-330 to about Val-340, and from about Val-348 to about Pro-366.

The data presented in FIGS. 5 and 6 are also represented in tabular form in Tables I and II, respectively. The columns are labeled with the headings “Res”, “Position”, and Roman Numerals I-XIV. The column headings refer to the following features of the amino acid sequence presented in FIGS. 5 and 6, and Tables I and II, respectively: “Res”: amino acid residue of SEQ ID NO:2 or FIGS. 2A and 2B (which is the identical sequence shown in SEQ ID NO:4, with the exception that the residues are numbered 1-366 in FIGS. 2A and 2B and −18 through 348 in SEQ ID NO:4); “Position”: position of the corresponding residue within SEQ ID NO:2 or FIGS. 2A and 2B (which is the identical sequence shown in SEQ ID NO:4, with the exception that the residues are numbered 1-366 in FIGS. 2A and 2B and −18 through 348 in SEQ ID NO:4); I: Alpha, Regions—Garnier-Robson; II: Alpha, Regions—Chou-Fasman; III: Beta, Regions—Garnier-Robson; IV: Beta, Regions—Chou-Fasman; V: Turn, Regions—Gamier-Robson; VI: Turn, Regions—Chou-Fasman; VII: Coil, Regions—Garnier-Robson; VII: Hydrophilicity Plot—Kyte-Doolittle; IX: Hydrophobicity Plot—Hopp-Woods; X: Alpha, Amphipathic Regions—Eisenberg; XI: Beta, Amphipathic Regions—Eisenberg; XII: Flexible Regions—Karplus-Schulz; XII: Antigenic Index—Jameson-Wolf; and XIV: Surface Probability Plot—Emini.

### DETAILED DESCRIPTION

The present invention provides isolated nucleic acid molecules comprising polynucleotides encoding a Nodal or Lefty polypeptide having the amino acid sequences shown in SEQ ID NO:2 and SEQ ID NO:4, respectively, which were determined by sequencing cloned cDNAs. The nucleotide sequences shown in FIGS. 1A and B and 2A and B (SEQ ID NO:1 and SEQ ID NO:3, respectively) were obtained by sequencing the HNGEF08 and HUKEJ46 clones, which were deposited on Jun. 5, 1997 at the American Type Culture Collection, 10801 University Boulevard, Manassas, Va. 20110-2209, and given accession numbers ATCC 209092 and 209135, and 209091, respectively. The deposited clones are contained in the pBluescript SK(-) plasmid (Stratagene, La Jolla, Calif.).

The Nodal and Lefty proteins of the present invention share sequence homology with the translation products of the murine mRNAs for Nodal and Lefty (FIGS. 3 and 4). Murine Nodal is thought to be an important TGF-β superfamily member involved in mesoderm formation during gastrulation (Zhou, X., et al., Nature 361:543-547 (1993)). During gastrulation, the three germ layers of the embryo are formed and organized along the anterior-posterior body axis. In addition, ectodermal cells of the primitive streak differentiate into the mesoderm. Murine Nodal was identified in mice which were homozygously mutated in the Nodal gene. A mutation in Nodal is prenatally lethal presumably due to the resulting gross developmental abnormalities.

Murine Lefty is involved in the developmental processes which establish lateral symmetry or handedness of the maturing embryonic organism (Meno, C., et al., Nature 381:151-155 (1996)). Lefty is believed to be a diffusible morphogen, the expression of which may result in the initiation of determination of symmetrical development in the mouse embryo. Lefty is transiently expressed in the left half of the gastrulating embryo just before the initiation of lateral symmetry.

### NUCLEIC ACID MOLECULES

Unless otherwise indicated, all nucleotide sequences determined by sequencing a DNA molecule herein were determined using an automated DNA sequencer (such as the Model 373 from Applied Biosystems, Inc., Foster City, Calif.), and all amino acid sequences of polypeptides encoded by DNA molecules determined herein were predicted by translation of a DNA sequence determined as above. Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

By “nucleotide sequence” of a nucleic acid molecule or polynucleotide is intended, for a DNA molecule or polynucleotide, a sequence of deoxyribonucleotides, and for an RNA molecule or polynucleotide, the corresponding sequence of ribonucleotides (A, G, C and U), where each thymidine deoxyribonucleotide (T) in the specified deoxyribonucleotide sequence is replaced by the ribonucleotide uridine (U).

Using the information provided herein, such as the nucleotide sequences in FIGS. 1A and B and 2A and B (SEQ ID NO:1 and SEQ ID NO:3, respectively), nucleic acid molecules of the present invention encoding a Nodal and Lefty polypeptide may be obtained using standard cloning and screening procedures, such as those for cloning cDNAs using mRNA as starting material. Illustrative of the invention, the nucleic acid molecules described in FIGS. 1A and B and 2A and B (SEQ ID NO:1 and SEQ ID NO:3, respectively) were discovered in cDNA libraries derived from neutrophils and uterine cancer, respectively. An additional clone of the Nodal gene was found in testis tissue. Additional clones of the Lefty gene were also identified in cDNA libraries from the following cell and tissue types: colon cancer, apoptotic T-cells, fetal heart, Wilm's Tumor tissue, frontal lobe of the brain from a patient with dementia, neutrophils, salivary gland, small intestine, 7, 8, and 12 week old human embryos, frontal cortex and hypothalamus from a patient with schizophrenia, brain from a patient with Alzheimer's Disease, adipose tissue, brown fat, TNF- and LPS-induced and uninduced bone marrow stroma, activated monocytes and macrophages, rhabdomyosarcoma, cycloheximide-treated Raji cells, breast lymph nodes, hemangiopericytoma, testes, fetal epithelium (skin), and IL-5-induced eosinophils.

Each of the determined nucleotide sequences of the Nodal and Lefty cDNAs shown in FIGS. 1A and B and 2A and B (SEQ ID NO:1 and SEQ ID NO:3, respectively) contains an open reading frame. The open reading frame found in FIGS. 1A-B encodes a protein of 283 amino acid residues, with an initiating aspartic acid codon at nucleotide positions 1-3 of the nucleotide sequence in FIG. 1A (SEQ ID NO:1), and a deduced molecular weight of about 32.5 kDa. The open reading frame found in FIGS. 2A-B encodes a protein of 366 amino acid residues, with an initiating methionine codon at nucleotide positions 53-55 of the nucleotide sequence in FIG. 2A (SEQ ID NO:3), and a deduced molecular weight of about 40.9 kDa. The amino acid sequence of the Nodal and Lefty proteins shown in SEQ ID NO:2 and SEQ ID NO:4, respectively, is about 80.9% and 82.0% identical to the murine mRNAs for Nodal and Lefty, respectively (FIGS. 3 and 4). The murine Nodal and Lefty genes have been described previously in the literature (Zhou, X., et al., Nature 361:543-547 (1993); Bouillet, P., et al., Dev. Biol. 170:420-433 (1995); Meno, C., et al., Nature 381:151-155 (1996)) and can be accessed on GenBank as Accession Nos. X70514 and Z73151, respectively.

The open reading frame of the Nodal gene shares sequence homology with the translation product of the murine mRNA for Nodal; FIG. 3; SEQ ID NO:3), particularly in the conserved active domain of about 110 amino acids. The open reading frame of the Lefty gene shares sequence homology with the translation product of the murine mRNA for Lefty; FIG. 4; SEQ ID NO:4), particularly in the conserved active domain of about 288 amino acids. Murine Nodal is thought to be important in correct mesoderm formation in the developing mouse embryo. Murine Lefty is thought to be important in the initiation of lateral a symmetry in the developing mouse embryo. The homologies between the murine Nodal and Lefty mRNAs and the novel human homologues of Nodal and Lefty indicate that the novel human homologues of Nodal and Lefty are involved in developmental roles as well as in the regulation of cell growth and differentiation. Further, it is likely that aberrant expression of Nodal and Lefty is a characteristic of cancer.

As members of the TGF-β superfamily, the novel human genes of the instant application also function in the regulation of immune and hematopoietic cell growth and differentiation.

As one of ordinary skill would appreciate, due to the possibilities of sequencing errors discussed above, the actual complete Nodal and Lefty polypeptides encoded by the deposited cDNAs, which comprise about 283 and 348 amino acids, respectively, may be somewhat longer or shorter. More generally, the actual open reading frame may be anywhere in the range of ±20 amino acids, more likely in the range of ±10 amino acids, of that predicted from either the codon at the N-terminus shown in FIGS. 1A and B and 2A and B (SEQ ID NO:1 and SEQ ID NO:3, respectively). It will further be appreciated that, depending on the analytical criteria used for identifying various functional domains, the exact “address” of the active domains of the Nodal and Lefty polypeptides may differ slightly from the predicted positions above.

Methods for predicting whether a protein has a secretory leader as well as the cleavage point for that leader sequence are known in the art and may routinely be applied to identify the leader sequence of the polynucleotides of the invention. For instance, the method of McGeoch (Virus Res. 3:271-286 (1985)) uses the information from a short N-terminal charged region and a subsequent uncharged region of the complete (uncleaved) protein. The method of von Heinje (Nucleic Acids Res. 14:4683-4690 (1986)) uses the information from the residues surrounding the cleavage site, typically residues −13 to +2 where +1 indicates the amino terminus of the mature protein. The accuracy of predicting the cleavage points of known mammalian secretory proteins for each of these methods is in the range of 75-80% (von Heinje, supra). However, the two methods do not always produce the same predicted cleavage point(s) for a given protein.

In the present case, the deduced amino acid sequences of the complete Nodal and Lefty polypeptides were analyzed by a computer program “PSORT”, available from Dr. Kenta Nakai of the Institute for Chemical Research, Kyoto University (Nakai, K. and Kanehisa, M. Genomics 14:897-911 (1992)), which is an expert system for predicting the cellular location of a protein based on the amino acid sequence. As part of this computational prediction of localization, the methods of McGeoch and von Heinje are incorporated.

In one embodiment, the computation analysis above predicted a single N-terminal signal sequence within the complete amino acid sequence shown in SEQ ID NO:4. Thus, the amino acid sequence of the complete Lefty protein includes a leader sequence and a mature protein, as shown in FIGS. 2A and 2B and SEQ ID NO:4. The amino acid sequence of the complete Nodal protein predicts a leader sequence and a mature protein, by comparison to the full-length murine Nodal ORF as shown in FIG. 3.

The present invention provides nucleic acid molecules encoding a mature form of the Lefty protein. According to the signal hypothesis, once export of the growing protein chain across the rough endoplasmic reticulum has been initiated, proteins secreted by mammalian cells have a signal or secretory leader sequence which is cleaved from the complete polypeptide to produce a secreted “mature” form of the protein. Most mammalian cells and even insect cells cleave secreted proteins with the same specificity. However, in some cases, cleavage of a secreted protein is not entirely uniform, which results in two or more mature species of the protein. Further, it has long been known that the cleavage specificity of a secreted protein is ultimately determined by the primary structure of the complete protein, that is, it is inherent in the amino acid sequence of the polypeptide. Therefore, the present invention provides a nucleotide sequence encoding the mature Lefty polypeptide having the amino acid sequence encoded by the cDNA clone contained in the host identified as ATCC Deposit No. 209091. By the “mature Lefty polypeptide having the amino acid sequence encoded by the cDNA clone in ATCC Deposit No. 209091” is meant the mature form(s) of the Lefty protein produced by expression in a mammalian cell (e.g., COS cells, as described below) of the complete open reading frame encoded by the human DNA sequence of the clone contained in the deposit.

Nucleic acid molecules of the present invention may be in the form of RNA, such as mRNA, or in the form of DNA, including, for instance, cDNA and genomic DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand or complementary strand.

In specific embodiments, the polynucleotides of the invention are less than 300 kb, 200 kb, 100 kb, 50 kb, 15 kb, 10 kb or 7.5 kb in length. In a further embodiment, polynucleotides of the invention comprise at least 15 contiguous nucleotides of Human Nodal or Human Lefty coding sequence, but do not comprise all or a portion of any Human Nodal or Human Lefty intron. In another embodiment, the nucleic acid comprising Human Nodal or Human Lefty coding sequence does not contain coding sequences of a genomic flanking gene (i.e., 5′ or 3′ to the Human Nodal or Human Lefty coding sequences in the genome).

By “isolated” nucleic acid molecule(s) is intended a nucleic acid molecule, DNA or RNA, which has been removed from its native environment For example, recombinant DNA molecules contained in a vector are considered isolated for the purposes of the present invention. Further examples of isolated DNA molecules include recombinant DNA molecules maintained in heterologous host cells or purified (partially or substantially) DNA molecules in solution. However, a nucleic acid contained in a clone that is a member of a library (e.g., a genomic or cDNA library) that has not been isolated from other members of the library (e.g., in the form of a homogeneous solution containing the clone and other members of the library) or which is contained on a chromosome preparation (e.g., a chromosome spread), is not “isolated” for the purposes of this invention. Isolated RNA molecules include in vivo or in vitro RNA transcripts of the DNA molecules of the present invention. Isolated nucleic acid molecules according to the present invention further include such molecules produced synthetically.

Isolated nucleic acid molecules of the present invention include DNA molecules comprising an open reading frame (ORF) with an initiating codon at positions 1-3 of the nucleotide sequence shown in FIG. 1A (SEQ ID NO:1) and DNA molecules comprising an open reading frame (ORF) with an initiation codon at positions 53-55 of the nucleotide sequence shown in FIG. 2A (SEQ ID NO:3).

Also included are DNA molecules comprising the coding sequence for the predicted mature Lefty protein shown at positions 1-366 of SEQ ID NO:4.

In addition, isolated nucleic acid molecules of the invention include DNA molecules which comprise a sequence substantially different from those described above, but, which, due to the degeneracy of the genetic code, still encode the Nodal or Lefty proteins. Of course, the genetic code and species-specific codon preferences are well known in the art. Thus, it would be routine for one skilled in the art to generate the degenerate variants described above, for instance, to optimize codon expression for a particular host (e.g., change codons in the human mRNA to those preferred by a bacterial host such as E. coli).

In another embodiment, the invention provides isolated nucleic acid molecules encoding the Nodal and Lefty polypeptides having amino acid sequences encoded by the cDNA clones contained in the plasmid deposited as ATCC Deposit Nos. 209092 and 209091 on Jun. 5, 1997 and the plasmid deposited as ATCC Deposit No. 209135 on Jul. 2, 1997.

Preferably, these nucleic acid molecules will encode the mature polypeptides encoded by the above-described deposited cDNA clones.

The invention further provides an isolated nucleic acid molecule having the nucleotide sequence shown in FIGS. 1A-B (SEQ ID NO:1) and an isolated nucleic acid molecule having the nucleotide sequence shown in FIGS. 2A-B (SEQ ID NO:3) or the nucleotide sequences of the Nodal and Lefty cDNAs contained in the above-described deposited clones, or a nucleic acid molecule having a sequence complementary to one of the above sequences. Such isolated molecules, particularly DNA molecules, are useful as probes for gene mapping, by in situ hybridization with chromosomes, and for detecting expression of the Nodal and Lefty genes in human tissue, for instance, by Northern blot analysis.

The present invention is further directed to nucleic acid molecules encoding portions of the nucleotide sequences described herein as well as to fragments of the isolated nucleic acid molecules described herein. In particular, the invention provides a polynucleotide having a nucleotide sequence representing the portion of SEQ ID NO:1 which consists of positions 1-852 of SEQ ID NO:1 and a polynucleotide having a nucleotide sequence representing the portion of SEQ ID NO:3 which consists of positions 1-1153 of SEQ ID NO:3. By a fragment of an isolated nucleic acid molecule having the nucleotide sequence of the deposited cDNAs (HTLFA20, HNGEF08, and HUKEJ46), or the nucleotide sequence shown in FIGS. 1A and B (SEQ ID NO:1), FIGS. 2A and B (SEQ ID NO:3), or the complementary strand thereto, is intended fragments at least 15 nt, and more preferably at least 20 nt, still more preferably at least 25 or 30 nt, and even more preferably, at least 40, 50, 60, 70, 80, 90, 100, 150, 200, 250, 300, 400, or 500 nt in length. These fragments have numerous uses which include, but are not limited to, diagnostic probes and primers as discussed herein. Of course, larger fragments 50-1500 nt in length are also useful according to the present invention as are fragments corresponding to most, if not all, of the nucleotide sequence of the deposited cDNA clone HTLFA20, the deposited cDNA clone HNGEF08, the deposited cDNA clone EUKEJ46, the nucleotide sequence depicted in FIGS. 1A and B (SEQ ID NO:1), or the nucleotide sequence depicted in FIGS. 2A and B (SEQ ID NO:4). By a fragment at least 20 nt in length, for example, is intended fragments which include 20 or more contiguous bases from the nucleotide sequence of the deposited cDNA clones (HTLFA20, HNGEF08, and HUKEJ46), the nucleotide sequence as shown in FIGS. 1A and B (SEQ ID NO:1) or the nucleotide sequence as shown in FIGS. 2A and B (SEQ ID NO:4).

In a preferred embodiment, the HUKEJ46 cDNA clone in ATCC Deposit No. 209091, which encodes the Human Lefty Homologue of the present invention, contains a cDNA insert which is represented by nucleotides 1-1596 of the sequence shown in FIGS. 2A and 2B.

In addition, the invention provides nucleic acid molecules having nucleotide sequences related to extensive portions of SEQ ID NO:3 which have been determined from the following related cDNA clones: HUKFN65R (SEQ ID NO:7) and HUKEJ46R (SEQ ID NO:8).

Further, the invention includes a polynucleotide comprising any portion of at least about 30 nucleotides, preferably at least about 50 nucleotides, of SEQ ID NO:1 from nucleotide 1-1130. More preferably, the invention includes a polynucleotide comprising nucleotides 250-1130, 500-1130, 750-1130, 1000-1130, 1-1000, 250-1000, 500-1000, 750-1000, 1-750, 250-750, 500-750, 1-500, 250-500, and 1-250 of SEQ ID NO:1. Likewise, the invention includes a polynucleotide comprising any portion of at least about 30 nucleotides, preferably at least about 50 nucleotides, of SEQ ID NO:3 from residue 1 to 950 and 1150 to 1688. More preferably, the invention includes a polynucleotide comprising nucleotides 250-1688, 500-1688, 750-1688, 1000-1688, 1250-1688, 1500-1688, 1-1500, 250-1500, 500-1500, 750-1500, 1000-1500, 1250-1500, 1-1250, 250-1250, 500-1250, 750-1250, 1000-1250, 1-1000, 250-1000, 500-1000, 750-1000, 1-750, 250-750, 500-750, 1-500, and 250-500 of SEQ ID NO:3.

Further specific embodiments are directed to polynucleotides corresponding to nucleotides 1-125, 1-90, 1-60, 1-30, 30-125, 30-90, 30-60, 60-125, 60-90, 90-125 310-930, 350-930, 400-930, 450-930, 500-930, 550-930, 600-930, 650-930, 700-930, 750-930, 800-930, 850-930, 900-930, 310-900, 350-900, 400-900, 450-900, 500-900, 550-900, 600-900, 650-900, 700-900, 750-900, 800-900, 850-900, 310-850, 350-850, 400-850, 450-850, 500-850, 550-850, 600-850, 650-850, 700-850, 750-850, 800-850, 310-800, 350-800, 400-800, 450-800, 500-800, 550-800, 600-800, 650-800, 700-800, 750-800, 310-750, 350-750, 400-750, 450-750, 500-750, 550-750, 600-750, 650-750, 700-750, 310-700, 350-700, 400-700, 450-700, 500-700, 550-700, 600-700, 650-700, 310-650, 350-650, 400-650, 450-650, 500-650, 550-650, 600-650, 310-600, 350-600, 400-600, 450-600, 500-600, 550-600, 310-500, 350-500, 400-500, 450-500, 310-450, 350-450, 400-450, 310-400, 350,-400, 310-350, 1050-1596, 1100-1596, 1150-1596, 1200-1596, 1250-1596, 1300-1596, 1350-1596, 1400-1596, 1450-1596, 1500-1596, 1550-1596, 1050-1550, 1100-1550, 1150-1550, 1200-1550, 1250-1550, 1300-1550, 1350-1550, 1400-1550, 1450-1550, 1500-1550, 1050-1500, 1100-1500, 1150-1500, 1200-1500, 1250-1500, 1300-1500, 1350-1500, 1400-1500, 1450-1500, 1050-1450, 1100-1450, 1150-1450, 1200-1450, 1250-1450, 1300-1450, 1350-1450, 1400-1450, 1050-1400, 1100-1400, 1150-1400, 1200-1400, 1250-1400, 1300-1400, 1350-1400, 1050-1350, 1100-1350, 1150-1350, 1200-1350, 1250-1350, 1300-1350, 1050-1300, 1100-1300, 1150-1300, 1200-1300, 1250-1300, 1050-1250, 1100-1250, 1150-1250, 1200-1250, 1050-1200, 1100-1200, 1150-1200, 1050-1150, 1100-1150, and 1050-1100 of SEQ ID NO:3.

More generally, by a fragment of an isolated nucleic acid molecule having the nucleotide sequence of the deposited cDNAs or the nucleotide sequences shown in FIGS. 1A and B and 2A and B (SEQ ID NO:1 and SEQ ID NO:3, respectively) is intended fragments at least about 15 nt, and more preferably at least about 20 nt, still more preferably at least about 25 nt or about 30 nt, and even more preferably, at least about 40 nt or about 45 nt in length which are useful as diagnostic probes and primers as discussed herein. Of course, larger fragments 50-300 nt in length are also useful according to the present invention as are fragments corresponding to most, if not all, of the nucleotide sequence of the deposited cDNAs or as shown in FIGS. 1A and B and 2A and B (SEQ ID NO:1 and SEQ ID NO:3, respectively). By a fragment at least 20 nt in length, for example, is intended fragments which include 20 or more contiguous bases from the nucleotide sequences of the deposited cDNAs or the nucleotide sequences as shown in FIGS. 1A and B and 2A and B (SEQ ID NO:1 and SEQ ID NO:3, respectively). By “about” in the phrase “at least about” is meant approximately and thus may refer to the identical number recited, or alternatively may differ in number by several, a few, or, alternatively, 5, 4, 3, 2 or 1 from the recited number. Preferred nucleic acid fragments of the present invention include nucleic acid molecules encoding epitope-bearing portions of the Nodal and Lefty polypeptides as identified in FIGS. 5 and 6 and described in more detail below.

In specific embodiments, the polynucleotide fragments of the invention encode a polypeptide which demonstrates a functional activity. By a polypeptide demonstrating “functional activity” is meant, a polypeptide capable of displaying one or more known functional activities associated with a complete, mature or TGF-β-like active forms of the Nodal or Lefty polypeptides. Such functional activities include, but are not limited to, biological activity ((e.g., the modulation of growth, development, and differentiation of a number of cell, tissue, and organ types (e.g., fibroblasts, keratinocytes, T- and B-lymphocytes, bone, cartilage, and other connective tissues, kidney, lung, and heart)), antigenicity [ability to bind (or compete with a Nodal or Lefty polypeptide for binding) to an anti-Nodal or anti-Lefty antibody], immunogenicity (ability to generate antibody which binds to a Nodal or Lefty polypeptide), the ability to form polymers (e.g., dimers) with other Nodal or Lefty or TGF-β polypeptides, and ability to bind to a receptor or ligand for a Nodal or Lefty polypeptide. These functional activities may routinely be determined using or routinely modifying techniques known in the art, such as, for example, immunoassays, etc.

Preferred nucleic acid fragments of the present invention also include nucleic acid molecules encoding one or more of the following domains of Nodal: amino acid residues 174-283 of SEQ ID NO:2 (i.e., the TGF-β-like domain of Nodal) and amino acid residues 1-27, 30-58, 64-82, 85-110, and 130-283 of SEQ ID NO:2. Preferred nucleic acid fragments of the present invention also include nucleic acid molecules encoding one or more of the following domains of Lefty: amino acid residues 1-348 of SEQ ID NO:4 (i.e., the mature domain of Lefty), amino acid residues 60-348 of SEQ ID NO:4 (i.e., the first predicted TGF-β-like domain of Lefty), amino acid residues 118-348 of SEQ ID NO:4 (i.e., the second predicted TGF-β-like domain of Lefty), amino acid residues 125-348 of SEQ ID NO:4 (i.e., the third predicted TGF-β-like domain of Lefty), and (−15)-(−2), 3-19, 34-51, 54-72, 75-114, 117-192, 198-209, 211-286, 290-302, and 305-348 of SEQ ID NO:4.

In specific embodiments, the polynucleotide fragments of the invention encode antigenic regions. Non-limiting examples of antigenic polypeptides or peptides that can be used to generate Nodal-specific antibodies include: a polypeptide comprising amino acid residues from about Lys-54 to about Asp-62, from about Val-91 to about Leu-99, from about Lys-100 to about Gln-108, from about Cys-116 to about Pro-124, from about Gln-140 to about Leu-148, from about Trp-156 to about Ser-164, from about Arg-170, to about Gln-181, from about Cys-212 to about Phe-224, from about Tyr-239, to about Thr-247, from about Pro-251, to about Met-259, and from about Asp-263, to about His-271. Non-limiting examples of antigenic polypeptides or peptides that can be used to generate Lefty-specific antibodies include: a polypeptide comprising amino acid residues from about Asp-71 to about Ser-79, from about Arg-106 to about Val-114, from about Leu-136 to about Arg-144, from about Asp-154 to about Asp-164, from about His-171 to about Asp-179, from about Gln-189 to about Leu-197, from about Pro-227 to about Glu-236, from about Gly-246 to about Glu-254, from about Pro-256 to about Gln-266, from about Cys-297 to about Ala-305, from about Ile-317 to about Pro-325, from about Ile-330 to about Val-340, and from about Val-348 to about Pro-366.

In additional embodiments, the polynucleotide fragments of the invention encode functional attributes of Human Nodal or Human Lefty. Preferred embodiments of the invention in this regard include fragments that comprise alpha-helix and alpha-helix forming regions (“alpha-regions”), beta-sheet and beta-sheet forming regions (“beta-regions”), turn and turn-forming regions (“turn-regions”), coil and coil-forming regions (“coil-regions”), hydrophilic regions, hydrophobic regions, alpha amphipathic regions, beta amphipathic regions, flexible regions, surface-forming regions and high antigenic index regions of Human Nodal or Human Lefty.

The data representing the structural or functional attributes of Nodal and Lefty set forth in FIGS. 5 and 6 and/or Tables I and II, as described above, was generated using the various modules and algorithms of the DNA*STAR set on default parameters. In a preferred embodiment, the data presented in columns VIII, IX, XIII, and XIV of Tables I and II can be used to determine regions of Nodal or Lefty which exhibit a high degree of potential for antigenicity. Regions of high antigenicity are determined from the data presented in columns VIII, IX, XIII, and/or IV by choosing values which represent regions of the polypeptide which are likely to be exposed on the surface of the polypeptide in an environment in which antigen recognition may occur in the process of initiation of an immune response.

Certain preferred regions in these regards are set out in FIGS. 5 and 6, but may, as shown in Tables I and II, respectively, be represented or identified by using tabular representations of the data presented in FIGS. 5 and 6. The DNA*STAR computer algorithm used to generate FIGS. 5 and 6 (set on the original default parameters) was used to present the data in FIGS. 5 and 6 in a tabular format (See Tables I and II, respectively). The tabular format of the data in FIG. 5 or in FIG. 6 may be used to easily determine specific boundaries of a preferred region.

The above-mentioned preferred regions set out in FIGS. 5 and 6 and in Tables I and II include, but are not limited to, regions of the aforementioned types identified by analysis of the amino acid sequence set out in FIGS. 1A and B and 2A and B. As set out in FIGS. 5 and 6 and in Tables I and II, such preferred regions include Garnier-Robson alpha-regions, beta-regions, turn-regions, and coil-regions, Chou-Fasman alpha-regions, beta-regions, and coil-regions, Kyte-Doolittle hydrophilic regions and hydrophobic regions, Eisenberg alpha- and beta-amphipathic regions, Karplus-Schulz flexible regions, Emini surface-forming regions and Jameson-Wolf regions of high antigenic index (generated using the amino acid sequences set out in FIGS. 1 and 2, and using the default parameters of the recited computer programs).

TABLE I

| Res | Position | I | II | III | IV | V | VI | VII | VIII | IX | X | XI | XII | XIII | XIV |
|---|---|---|---|---|---|---|---|---|---|---|---|---|---|---|---|
| Asp | 1 | . | . | B | . | . | . | . | −0.36 | 0.07 | . | * | . | −0.10 | 0.35 |
| Val | 2 | . | . | B | . | . | . | . | −0.31 | −0.36 | . | * | . | 0.50 | 0.45 |
| Ala | 3 | . | . | B | . | . | . | . | 0.08 | −0.36 | . | * | . | 0.50 | 0.35 |
| Val | 4 | . | . | B | . | . | . | . | 0.47 | −0.39 | . | * | . | 0.50 | 0.37 |
| Asp | 5 | . | . | B | . | . | . | . | 0.57 | 0.01 | . | * | F | 0.05 | 0.79 |
| Gly | 6 | . | . | . | . | T | T | . | 0.26 | 0.29 | . | * | F | 0.65 | 0.82 |
| Gln | 7 | . | . | . | . | . | T | C | 0.41 | 0.27 | . | . | F | 0.60 | 1.60 |
| Asn | 8 | . | . | . | . | T | T | . | 0.41 | 0.41 | . | . | F | 0.35 | 0.83 |
| Trp | 9 | . | . | B | . | . | T | . | 0.57 | 0.91 | . | * | . | −0.20 | 0.85 |
| Thr | 10 | . | A | B | . | . | . | . | 0.57 | 1.27 | . | * | . | −0.60 | 0.42 |
| Phe | 11 | . | A | B | . | . | . | . | 0.21 | 0.87 | . | * | . | −0.60 | 0.44 |
| Ala | 12 | . | A | B | . | . | . | . | −0.09 | 1.26 | . | * | . | −0.60 | 0.36 |
| Phe | 13 | . | A | B | . | . | . | . | −0.79 | 0.73 | . | * | . | −0.60 | 0.34 |
| Asp | 14 | . | A | . | . | T | . | . | −1.31 | 1.03 | . | * | . | −0.20 | 0.34 |
| Phe | 15 | . | A | . | . | T | . | . | −1.30 | 0.93 | . | * | . | −0.20 | 0.27 |
| Ser | 16 | . | A | . | . | . | . | C | −0.60 | 0.81 | . | * | . | −0.40 | 0.43 |
| Phe | 17 | A | A | . | . | . | . | . | −0.01 | 0.43 | . | * | . | −0.60 | 0.44 |
| Leu | 18 | A | A | . | . | . | . | . | 0.69 | 0.83 | . | * | . | −0.60 | 0.88 |
| Ser | 19 | A | A | . | . | . | . | . | 0.69 | 0.04 | . | . | F | 0.00 | 1.14 |
| Gln | 20 | A | A | . | . | . | . | . | 0.58 | −0.34 | . | . | F | 0.60 | 2.20 |
| Gln | 21 | A | A | . | . | . | . | . | 0.29 | −0.44 | . | . | F | 0.60 | 2.20 |
| Glu | 22 | A | A | . | . | . | . | . | 0.70 | −0.63 | . | . | F | 0.90 | 1.66 |
| Asp | 23 | A | A | . | . | . | . | . | 0.92 | −0.10 | . | . | F | 0.60 | 1.01 |
| Leu | 24 | A | A | . | . | . | . | . | 1.22 | 0.00 | . | . | . | −0.30 | 0.59 |
| Ala | 25 | A | A | . | . | . | . | . | 0.41 | −0.40 | . | * | . | 0.30 | 0.59 |
| Trp | 26 | A | A | . | . | . | . | . | 0.52 | 0.29 | . | * | . | −0.30 | 0.29 |
| Ala | 27 | A | A | . | . | . | . | . | −0.29 | 0.29 | . | * | . | −0.30 | 0.69 |
| Glu | 28 | A | A | . | . | . | . | . | −0.29 | 0.29 | . | * | . | −0.30 | 0.56 |
| Leu | 29 | A | A | . | . | . | . | . | −0.29 | 0.19 | . | * | . | −0.30 | 0.93 |
| Arg | 30 | A | A | . | . | . | . | . | −0.00 | −0.04 | . | * | . | 0.30 | 0.76 |
| Leu | 31 | A | A | . | . | . | . | . | −0.01 | −0.16 | . | * | . | 0.30 | 0.58 |
| Gln | 32 | . | A | . | . | T | . | . | 0.37 | 0.23 | . | * | . | 0.10 | 0.95 |
| Leu | 33 | . | A | . | . | T | . | . | −0.49 | −0.03 | . | * | . | 0.70 | 0.75 |
| Ser | 34 | . | A | . | . | . | . | C | 0.32 | 0.61 | . | * | F | −0.25 | 0.67 |
| Ser | 35 | . | . | . | . | . | T | C | −0.60 | −0.07 | . | * | F | 1.05 | 0.65 |
| Pro | 36 | . | . | B | . | . | T | . | 0.00 | 0.21 | . | * | F | 0.25 | 0.65 |
| Val | 37 | . | . | B | . | . | T | . | −0.31 | −0.04 | * | * | F | 0.85 | 0.75 |
| Asp | 38 | . | . | B | . | . | T | . | 0.50 | 0.06 | * | * | F | 0.25 | 0.81 |
| Leu | 39 | . | . | B | . | . | . | . | 0.46 | −0.33 | . | * | F | 0.65 | 0.91 |
| Pro | 40 | . | . | B | . | . | T | . | 0.46 | −0.33 | . | * | F | 1.00 | 1.21 |
| Thr | 41 | A | . | . | . | . | T | . | −0.14 | −0.59 | . | * | F | 1.15 | 0.97 |
| Glu | 42 | A | . | . | . | . | T | . | 0.12 | 0.10 | . | * | F | 0.25 | 0.97 |
| Gly | 43 | A | . | . | . | . | T | . | −0.77 | −0.09 | * | * | F | 0.85 | 0.63 |
| Ser | 44 | A | A | . | . | . | . | . | 0.04 | 0.17 | . | * | F | −0.15 | 0.31 |
| Leu | 45 | A | A | . | . | . | . | . | −0.63 | −0.31 | . | * | . | 0.30 | 0.31 |
| Ala | 46 | A | A | . | . | . | . | . | −1.02 | 0.37 | . | * | . | −0.30 | 0.22 |
| Ile | 47 | A | A | . | . | . | . | . | −1.06 | 0.73 | * | * | . | −0.60 | 0.14 |
| Glu | 48 | A | A | . | . | . | . | . | −0.71 | 0.84 | * | * | . | −0.60 | 0.23 |
| Ile | 49 | A | A | . | . | . | . | . | −0.62 | 0.56 | * | * | . | −0.60 | 0.40 |
| Phe | 50 | . | A | B | . | . | . | . | 0.23 | 0.49 | * | * | . | −0.60 | 0.88 |
| His | 51 | . | A | . | . | . | . | C | 0.61 | −0.20 | * | * | . | 0.65 | 1.01 |
| Gln | 52 | . | A | . | . | . | . | C | 1.50 | 0.23 | . | * | F | 0.54 | 2.24 |
| Pro | 53 | . | A | . | . | . | . | C | 1.19 | −0.46 | * | * | F | 1.48 | 4.31 |
| Lys | 54 | . | . | . | . | . | T | C | 2.08 | −0.76 | . | . | F | 2.52 | 4.58 |
| Pro | 55 | . | . | . | . | . | T | C | 2.78 | −1.26 | . | . | F | 2.86 | 4.58 |
| Asp | 56 | . | . | . | . | T | T | . | 2.22 | −1.26 | . | . | F | 3.40 | 5.12 |
| Thr | 57 | A | . | . | . | . | T | . | 1.92 | −1.19 | . | . | F | 2.66 | 2.59 |
| Glu | 58 | A | . | . | . | . | . | . | 2.13 | −0.80 | . | . | F | 2.12 | 2.24 |
| Gln | 59 | A | . | . | . | . | . | . | 1.79 | −1.23 | . | . | F | 1.78 | 2.24 |
| Ala | 60 | A | . | . | . | . | . | . | 1.33 | −0.84 | . | . | F | 1.44 | 2.08 |
| Ser | 61 | A | . | . | . | . | T | . | 0.52 | −0.76 | * | . | F | 1.15 | 0.64 |
| Asp | 62 | A | . | . | . | . | T | . | 0.83 | −0.07 | * | . | F | 0.85 | 0.31 |
| Ser | 63 | A | . | . | . | . | T | . | 0.94 | −0.47 | * | . | F | 0.85 | 0.53 |
| Cys | 64 | A | . | . | . | . | T | . | 0.24 | −0.97 | * | . | . | 1.00 | 0.77 |
| Leu | 65 | A | A | . | . | . | . | . | 0.83 | −0.57 | * | * | . | 0.60 | 0.40 |
| Glu | 66 | A | A | . | . | . | . | . | 0.53 | −0.17 | * | * | . | 0.30 | 0.52 |
| Arg | 67 | A | A | . | . | . | . | . | 0.53 | 0.06 | * | * | . | −0.30 | 0.95 |
| Phe | 68 | A | A | . | . | . | . | . | 0.02 | −0.51 | * | * | . | 0.75 | 1.93 |
| Gln | 69 | A | A | . | . | . | . | . | −0.01 | −0.51 | * | * | . | 0.60 | 0.92 |
| Met | 70 | A | . | . | B | . | . | . | 0.49 | 0.27 | * | * | . | −0.30 | 0.41 |
| Asp | 71 | A | . | . | B | . | . | . | −0.37 | 0.76 | * | * | . | −0.60 | 0.68 |
| Leu | 72 | A | . | . | B | . | . | . | −0.79 | 0.61 | * | * | . | −0.60 | 0.29 |
| Phe | 73 | . | . | B | B | . | . | . | −0.90 | 0.70 | . | * | . | −0.60 | 0.42 |
| Thr | 74 | . | . | B | B | . | . | . | −1.20 | 0.77 | . | . | . | −0.60 | 0.21 |
| Val | 75 | . | . | B | B | . | . | . | −0.60 | 1.16 | * | . | . | −0.60 | 0.34 |
| Thr | 76 | . | . | B | B | . | . | . | −1.46 | 0.87 | * | . | . | −0.60 | 0.68 |
| Leu | 77 | . | . | B | B | . | . | . | −0.96 | 0.73 | . | . | . | −0.60 | 0.35 |
| Ser | 78 | . | . | B | B | . | . | . | −0.96 | 0.73 | . | * | . | −0.60 | 0.68 |
| Gln | 79 | . | . | B | B | . | . | . | −0.94 | 0.87 | . | * | . | −0.60 | 0.41 |
| Val | 80 | . | . | B | B | . | . | . | −0.90 | 0.77 | . | . | . | −0.60 | 0.66 |
| Thr | 81 | . | . | B | B | . | . | . | −0.93 | 0.77 | . | . | . | −0.60 | 0.41 |
| Phe | 82 | . | . | B | B | . | . | . | −0.42 | 0.81 | * | . | . | −0.60 | 0.23 |
| Ser | 83 | . | . | B | . | . | . | . | −0.72 | 0.80 | . | * | . | −0.40 | 0.42 |
| Leu | 84 | . | . | B | . | . | . | . | −1.58 | 0.77 | . | * | . | −0.40 | 0.29 |
| Gly | 85 | . | . | . | B | . | . | C | −1.53 | 0.93 | . | . | . | −0.40 | 0.25 |
| Ser | 86 | . | . | . | B | . | . | C | −1.22 | 0.83 | . | . | . | −0.40 | 0.15 |
| Met | 87 | . | . | B | B | . | . | . | −1.38 | 0.44 | . | . | . | −0.60 | 0.32 |
| Val | 88 | . | . | B | B | . | . | . | −1.39 | 0.40 | * | . | . | −0.60 | 0.24 |
| Leu | 89 | . | . | B | B | . | . | . | −0.47 | 0.46 | * | . | . | −0.60 | 0.26 |
| Glu | 90 | . | . | B | B | . | . | . | −0.33 | 0.07 | * | . | . | −0.30 | 0.51 |
| Val | 91 | . | . | B | B | . | . | . | −0.84 | −0.11 | * | . | . | 0.45 | 1.06 |
| Thr | 92 | A | . | . | B | . | . | . | −0.54 | −0.07 | * | . | F | 0.60 | 1.06 |
| Arg | 93 | A | . | . | . | . | T | . | 0.36 | −0.37 | * | . | F | 0.85 | 0.82 |
| Pro | 94 | A | . | . | . | . | T | . | 0.88 | −0.37 | * | . | F | 1.00 | 2.21 |
| Leu | 95 | A | . | . | . | . | T | . | 0.07 | −0.10 | * | . | F | 1.00 | 1.61 |
| Ser | 96 | A | . | . | . | . | T | . | 0.97 | 0.10 | * | . | F | 0.25 | 0.68 |
| Lys | 97 | . | A | . | . | T | . | . | 1.39 | 0.10 | * | . | F | 0.49 | 0.88 |
| Trp | 98 | . | A | B | . | . | . | . | 1.07 | −0.33 | * | . | F | 1.08 | 2.09 |
| Leu | 99 | . | A | B | . | . | . | . | 0.93 | −0.59 | * | . | F | 1.62 | 2.41 |
| Lys | 100 | . | A | B | . | . | . | . | 1.16 | −0.54 | * | . | F | 1.86 | 1.19 |
| Arg | 101 | . | . | . | . | . | T | C | 0.64 | −0.04 | * | . | F | 2.40 | 1.14 |
| Pro | 102 | . | . | . | . | . | T | C | 0.60 | −0.27 | * | . | F | 2.16 | 1.14 |
| Gly | 103 | . | . | . | . | . | T | C | 0.93 | −0.96 | * | . | F | 2.07 | 0.99 |
| Ala | 104 | A | . | . | . | . | T | . | 1.74 | −0.96 | * | . | F | 1.78 | 1.01 |
| Leu | 105 | A | A | . | . | . | . | . | 1.10 | −0.56 | * | . | F | 1.14 | 1.13 |
| Glu | 106 | A | A | . | . | . | . | . | 0.69 | −0.37 | * | . | F | 0.60 | 1.13 |
| Lys | 107 | A | A | . | . | . | . | . | 1.01 | −0.41 | * | . | F | 0.60 | 1.50 |
| Gln | 108 | A | A | . | . | . | . | . | 0.50 | −0.91 | * | . | F | 0.90 | 3.57 |
| Met | 109 | A | A | . | . | . | . | . | 0.50 | −0.96 | * | . | F | 0.90 | 1.53 |
| Ser | 110 | A | A | . | . | . | . | . | 0.97 | −0.46 | * | * | F | 0.45 | 0.77 |
| Arg | 111 | . | A | B | . | . | . | . | 0.97 | −0.03 | * | * | . | 0.30 | 0.44 |
| Val | 112 | . | A | B | . | . | . | . | 0.26 | −0.43 | * | * | . | 0.30 | 0.77 |
| Ala | 113 | . | A | . | . | T | . | . | −0.03 | −0.47 | * | . | . | 0.70 | 0.31 |
| Gly | 114 | . | A | . | . | T | . | . | 0.36 | 0.06 | * | * | . | 0.35 | 0.17 |
| Glu | 115 | . | A | . | . | T | . | . | 0.77 | 0.49 | * | * | . | 0.30 | 0.35 |
| Cys | 116 | . | A | . | . | T | . | . | 0.44 | −0.16 | * | * | . | 1.45 | 0.67 |
| Trp | 117 | . | . | . | . | T | T | . | 1.09 | −0.23 | * | * | . | 2.25 | 1.05 |
| Pro | 118 | . | . | . | . | T | T | . | 1.37 | −0.23 | * | . | F | 2.50 | 0.94 |
| Arg | 119 | . | . | . | . | . | T | C | 1.50 | 0.26 | * | . | F | 1.60 | 2.52 |
| Pro | 120 | . | . | . | . | . | T | C | 1.29 | 0.11 | * | . | F | 1.35 | 3.70 |
| Pro | 121 | . | . | . | . | T | . | . | 1.37 | −0.37 | * | . | F | 1.70 | 3.70 |
| Thr | 122 | . | . | . | . | . | . | C | 1.34 | −0.30 | * | . | F | 1.25 | 1.91 |
| Pro | 123 | . | . | . | . | . | T | C | 1.56 | 0.19 | * | . | F | 0.60 | 1.78 |
| Pro | 124 | . | . | . | . | . | T | C | 0.59 | 0.16 | * | . | F | 0.60 | 1.85 |
| Ala | 125 | . | . | B | . | . | T | . | −0.01 | 0.37 | . | . | F | 0.25 | 0.95 |
| Thr | 126 | . | . | B | . | . | T | . | −0.61 | 0.57 | . | . | F | −0.05 | 0.51 |
| Asn | 127 | . | A | B | . | . | . | . | −0.90 | 0.83 | . | . | . | −0.60 | 0.27 |
| Val | 128 | . | A | B | . | . | . | . | −1.50 | 1.01 | . | . | . | −0.60 | 0.27 |
| Leu | 129 | . | A | B | . | . | . | . | −1.53 | 1.20 | . | . | . | −0.60 | 0.15 |
| Leu | 130 | . | A | B | . | . | . | . | −1.24 | 1.47 | . | . | . | −0.60 | 0.15 |
| Met | 131 | . | A | B | . | . | . | . | −0.93 | 1.46 | * | . | . | −0.60 | 0.27 |
| Leu | 132 | . | A | B | . | . | . | . | −1.74 | 1.21 | * | . | . | −0.60 | 0.52 |
| Tyr | 133 | . | . | B | . | . | T | . | −1.19 | 1.21 | * | . | . | −0.20 | 0.52 |
| Ser | 134 | . | . | . | . | . | T | C | −0.38 | 0.91 | * | . | . | 0.00 | 0.71 |
| Asn | 135 | . | . | . | . | . | T | C | 0.43 | 0.70 | . | . | F | 0.30 | 1.48 |
| Leu | 136 | . | . | . | . | . | T | C | 1.03 | 0.01 | * | * | F | 0.60 | 1.64 |
| Ser | 137 | A | A | . | . | . | . | . | 1.96 | −0.34 | * | . | F | 0.60 | 2.12 |
| Gln | 138 | A | A | . | . | . | . | . | 2.20 | −0.73 | * | . | F | 0.90 | 2.58 |
| Glu | 139 | . | A | B | . | . | . | . | 1.69 | −0.73 | * | . | F | 0.90 | 5.41 |
| Gln | 140 | . | A | B | . | . | . | . | 1.34 | −0.73 | * | . | F | 1.15 | 3.33 |
| Arg | 141 | . | A | B | . | . | . | . | 1.81 | −0.69 | * | . | F | 1.40 | 1.90 |
| Gln | 142 | . | A | B | . | . | . | . | 1.81 | −0.66 | . | . | F | 1.65 | 1.09 |
| Leu | 143 | . | . | . | . | T | T | . | 1.50 | −0.27 | . | . | F | 2.25 | 0.84 |
| Gly | 144 | . | . | . | . | T | T | . | 0.69 | −0.19 | . | . | F | 2.50 | 0.62 |
| Gly | 145 | . | . | . | . | . | T | C | −0.12 | 0.50 | . | . | F | 1.15 | 0.30 |
| Ser | 146 | . | . | . | . | . | T | C | −0.52 | 0.79 | . | . | F | 0.90 | 0.30 |
| Thr | 147 | . | A | . | . | . | . | C | −0.52 | 1.01 | . | . | F | 0.25 | 0.31 |
| Leu | 148 | . | A | B | . | . | . | . | −0.30 | 0.59 | . | . | F | −0.20 | 0.55 |
| Leu | 149 | . | A | B | . | . | . | . | 0.04 | 0.66 | . | . | . | −0.60 | 0.41 |
| Trp | 150 | A | A | . | . | . | . | . | 0.09 | 0.27 | . | . | . | −0.30 | 0.50 |
| Glu | 151 | A | A | . | . | . | . | . | 0.09 | 0.17 | . | * | . | −0.30 | 0.81 |
| Ala | 152 | A | A | . | . | . | . | . | 0.11 | −0.13 | * | * | F | 0.60 | 1.31 |
| Glu | 153 | A | . | . | . | . | T | . | 1.03 | 0.10 | * | * | F | 0.40 | 1.31 |
| Ser | 154 | A | . | . | . | . | T | . | 1.26 | −0.81 | . | * | F | 1.30 | 1.48 |
| Ser | 155 | A | . | . | . | . | T | . | 1.54 | −0.31 | . | * | F | 1.00 | 1.48 |
| Trp | 156 | A | . | . | . | . | T | . | 1.54 | −0.41 | . | * | F | 1.23 | 1.48 |
| Arg | 157 | A | . | . | . | . | . | . | 1.79 | −0.41 | . | * | . | 1.11 | 1.92 |
| Ala | 158 | A | . | . | . | . | . | . | 1.79 | −0.37 | . | * | F | 1.49 | 1.42 |
| Gln | 159 | A | . | . | . | . | . | . | 1.28 | −0.36 | . | * | F | 1.72 | 2.33 |
| Glu | 160 | . | . | . | . | . | . | C | 1.28 | −0.59 | . | * | F | 2.30 | 0.98 |
| Gly | 161 | . | . | . | . | . | . | C | 1.28 | −0.20 | . | * | F | 1.92 | 1.30 |
| Gln | 162 | . | . | . | . | . | . | C | 1.17 | 0.21 | . | * | F | 0.94 | 0.79 |
| Leu | 163 | . | . | . | . | . | . | C | 1.47 | −0.19 | . | * | . | 1.16 | 0.79 |
| Ser | 164 | . | . | . | . | . | . | C | 1.12 | 0.73 | * | * | . | 0.03 | 0.84 |
| Trp | 165 | A | . | . | . | . | . | . | 1.17 | 0.73 | * | * | . | −0.40 | 0.48 |
| Glu | 166 | A | . | . | . | . | . | . | 1.62 | 0.33 | * | * | . | 0.35 | 1.16 |
| Trp | 167 | A | . | . | . | . | . | . | 1.59 | −0.36 | * | * | . | 1.25 | 1.70 |
| Gly | 168 | A | . | . | . | . | . | . | 2.51 | −0.24 | * | * | F | 1.70 | 2.20 |
| Lys | 169 | . | . | . | . | T | . | . | 2.92 | −1.16 | * | . | F | 2.70 | 2.49 |
| Arg | 170 | . | . | . | . | T | . | . | 3.18 | −1.16 | * | . | F | 3.00 | 4.64 |
| His | 171 | . | . | . | . | T | . | . | 3.14 | −1.57 | * | . | F | 2.70 | 6.38 |
| Arg | 172 | . | . | . | . | T | . | . | 2.62 | −1.50 | * | . | F | 2.40 | 4.34 |
| Arg | 173 | . | . | . | . | T | . | . | 2.76 | −0.81 | . | . | . | 1.95 | 1.83 |
| His | 174 | . | . | . | . | T | . | . | 2.71 | −0.39 | . | . | . | 1.69 | 2.08 |
| His | 175 | . | . | . | . | . | . | C | 2.71 | −0.89 | . | * | . | 1.83 | 1.77 |
| Leu | 176 | . | . | . | . | . | T | C | 2.44 | −0.89 | . | * | . | 2.37 | 1.77 |
| Pro | 177 | . | . | . | . | T | T | . | 2.33 | −0.50 | . | * | F | 2.76 | 1.74 |
| Asp | 178 | . | . | . | . | T | T | . | 1.41 | −0.60 | . | * | F | 3.40 | 2.22 |
| Arg | 179 | . | . | . | . | T | T | . | 0.78 | −0.41 | . | . | F | 2.76 | 2.22 |
| Ser | 180 | A | . | . | B | . | . | . | 0.92 | −0.53 | . | * | F | 1.77 | 0.77 |
| Gln | 181 | A | . | . | B | . | . | . | 1.78 | −0.96 | * | * | F | 1.43 | 0.90 |
| Leu | 182 | A | . | . | B | . | . | . | 1.13 | −0.96 | * | * | F | 1.09 | 0.92 |
| Cys | 183 | . | . | B | B | . | . | . | 1.18 | −0.31 | . | * | . | 0.30 | 0.51 |
| Arg | 184 | . | . | B | B | . | . | . | 0.37 | −0.70 | . | * | . | 0.60 | 0.59 |
| Lys | 185 | . | . | B | B | . | . | . | 0.67 | −0.31 | * | * | F | 0.45 | 0.62 |
| Val | 186 | . | . | B | B | . | . | . | −0.19 | −0.60 | * | * | F | 0.90 | 2.00 |
| Lys | 187 | . | . | B | B | . | . | . | 0.62 | −0.53 | * | * | . | 0.60 | 0.76 |
| Phe | 188 | . | . | B | B | . | . | . | 0.59 | −0.53 | . | * | . | 0.60 | 0.63 |
| Gln | 189 | . | . | B | B | . | . | . | 0.48 | 0.26 | . | * | . | −0.30 | 0.74 |
| Val | 190 | . | . | B | B | . | . | . | −0.38 | 0.01 | . | * | . | −0.30 | 0.59 |
| Asp | 191 | . | . | B | B | . | . | . | −0.41 | 0.70 | . | * | . | −0.60 | 0.57 |
| Phe | 192 | . | . | B | B | . | − | . | −0.80 | 0.60 | . | * | . | −0.60 | 0.23 |
| Asn | 193 | . | . | B | B | . | . | . | −0.39 | 0.63 | . | * | . | −0.60 | 0.30 |
| Leu | 194 | . | . | B | B | . | . | . | −0.73 | 0.90 | . | * | . | −0.60 | 0.19 |
| Ile | 195 | . | . | . | B | . | . | C | −0.18 | 1.33 | . | * | . | −0.40 | 0.22 |
| Gly | 196 | . | . | . | B | T | . | . | −0.47 | 0.93 | . | . | . | −0.20 | 0.18 |
| Trp | 197 | . | . | . | . | T | T | . | −0.66 | 1.44 | . | . | . | 0.20 | 0.23 |
| Gly | 198 | . | . | . | . | . | T | C | −1.54 | 1.44 | . | . | . | 0.00 | 0.23 |
| Ser | 199 | . | . | . | . | T | T | . | −0.98 | 1.44 | . | . | . | 0.20 | 0.17 |
| Trp | 200 | . | . | B | . | . | T | . | −0.30 | 1.77 | . | . | . | −0.20 | 0.25 |
| Ile | 201 | . | . | B | . | . | . | . | 0.09 | 1.29 | . | . | . | −0.40 | 0.38 |
| Ile | 202 | . | . | B | . | . | . | . | 0.38 | 0.86 | . | . | . | −0.40 | 0.57 |
| Tyr | 203 | . | . | B | . | . | T | . | 0.48 | 0.87 | . | . | . | −0.20 | 0.95 |
| Pro | 204 | . | . | . | . | T | T | . | 0.78 | 0.71 | . | . | F | 0.50 | 2.11 |
| Lys | 205 | . | . | . | . | T | T | . | 0.48 | 0.43 | . | . | F | 0.50 | 4.85 |
| Gln | 206 | . | . | . | . | T | T | . | 1.12 | 0.24 | . | . | F | 0.80 | 3.13 |
| Tyr | 207 | . | . | . | . | T | . | . | 2.12 | 0.24 | * | . | . | 0.45 | 3.17 |
| Asn | 208 | . | . | . | . | T | T | . | 1.70 | −0.19 | . | . | . | 1.25 | 3.10 |
| Ala | 209 | . | . | B | . | . | T | . | 1.91 | 0.39 | . | . | . | 0.37 | 0.96 |
| Tyr | 210 | . | . | B | . | . | T | . | 1.52 | −0.01 | . | * | . | 1.39 | 1.06 |
| Arg | 211 | . | . | B | . | . | T | . | 1.52 | −0.34 | . | * | . | 1.51 | 0.65 |
| Cys | 212 | . | . | B | . | . | . | . | 1.10 | −0.74 | * | * | . | 2.03 | 1.12 |
| Glu | 213 | . | . | . | . | T | . | . | 0.89 | −0.67 | * | * | F | 2.70 | 0.38 |
| Gly | 214 | . | . | . | . | T | . | . | 1.48 | −1.00 | * | * | F | 2.43 | 0.30 |
| Glu | 215 | . | . | . | . | T | . | . | 1.51 | −0.60 | * | * | F | 2.16 | 0.91 |
| Cys | 216 | . | . | . | . | . | T | C | 0.54 | −0.74 | * | * | F | 2.15 | 0.81 |
| Pro | 217 | . | . | . | . | . | T | C | 0.87 | −0.10 | . | . | F | 1.84 | 0.61 |
| Asn | 218 | . | . | . | . | . | T | C | 0.87 | −0.10 | . | . | F | 1.83 | 0.35 |
| Pro | 219 | . | . | . | . | . | T | C | 1.21 | −0.10 | * | . | F | 2.24 | 1.12 |
| Val | 220 | . | . | . | . | . | . | C | 0.51 | −0.67 | * | . | F | 2.60 | 1.26 |
| Gly | 221 | A | . | . | . | . | . | . | 1.14 | −0.31 | * | . | F | 1.69 | 0.68 |
| Glu | 222 | A | . | . | . | . | . | . | 1.14 | −0.21 | * | . | F | 1.43 | 0.60 |
| Glu | 223 | A | . | . | . | . | . | . | 0.83 | −0.21 | * | . | F | 1.42 | 1.24 |
| Phe | 224 | A | . | . | . | . | . | . | 1.04 | −0.37 | . | . | F | 1.26 | 1.81 |
| His | 225 | A | . | . | . | . | T | . | 1.87 | −0.40 | . | . | F | 1.30 | 1.68 |
| Pro | 226 | A | . | . | . | . | T | . | 1.62 | 0.10 | . | . | F | 0.80 | 1.32 |
| Thr | 227 | . | . | . | . | T | T | . | 1.38 | 0.60 | . | . | F | 1.00 | 1.54 |
| Asn | 228 | A | . | . | . | . | T | . | 0.49 | 0.57 | . | * | . | 0.35 | 1.77 |
| His | 229 | A | . | . | B | . | . | . | 1.19 | 0.76 | . | . | . | −0.30 | 0.80 |
| Ala | 230 | A | . | . | B | . | . | . | 0.92 | 0.73 | . | . | . | −0.40 | 0.96 |
| Tyr | 231 | A | . | . | B | . | . | . | 0.32 | 0.63 | . | . | . | −0.50 | 0.80 |
| Ile | 232 | . | . | B | B | . | . | . | −0.18 | 0.91 | * | * | . | −0.60 | 0.49 |
| Gln | 233 | . | . | B | B | . | . | . | −0.13 | 1.10 | * | . | . | −0.60 | 0.40 |
| Ser | 234 | . | . | B | B | . | . | . | 0.01 | 0.60 | * | . | . | −0.60 | 0.51 |
| Leu | 235 | . | . | B | B | . | . | . | 0.36 | −0.16 | * | . | F | 0.60 | 1.42 |
| Leu | 236 | . | . | B | B | . | . | . | 0.60 | −0.09 | * | . | F | 0.60 | 1.28 |
| Lys | 237 | . | . | . | B | T | . | . | 1.28 | −0.09 | * | . | F | 1.00 | 1.66 |
| Arg | 238 | . | . | . | . | T | . | . | 1.24 | −0.04 | . | . | F | 1.20 | 3.11 |
| Tyr | 239 | . | . | B | . | . | . | . | 1.66 | −0.23 | . | . | F | 1.08 | 5.13 |
| Gln | 240 | . | . | B | . | . | T | . | 1.61 | −0.91 | . | . | F | 1.86 | 5.02 |
| Pro | 241 | . | . | B | . | . | T | . | 2.21 | −0.27 | . | . | F | 1.84 | 1.90 |
| His | 242 | . | . | . | . | T | T | . | 1.87 | 0.16 | . | . | . | 1.77 | 1.88 |
| Arg | 243 | . | . | . | . | T | T | . | 1.44 | −0.21 | . | . | F | 2.80 | 1.45 |
| Val | 244 | . | . | B | . | . | . | . | 1.02 | −0.13 | * | . | F | 1.92 | 1.36 |
| Pro | 245 | . | . | . | . | T | . | . | 0.36 | 0.01 | * | . | F | 1.29 | 0.53 |
| Ser | 246 | . | . | . | . | T | T | . | −0.02 | 0.09 | * | * | F | 1.21 | 0.15 |
| Thr | 247 | . | . | . | . | T | T | . | −0.20 | 0.59 | * | * | F | 0.63 | 0.20 |
| Cys | 248 | . | . | B | . | . | T | . | −1.17 | 0.37 | * | * | . | 0.10 | 0.20 |
| Cys | 249 | . | . | B | . | . | T | . | −0.27 | 0.59 | . | * | . | −0.20 | 0.11 |
| Ala | 250 | . | . | B | . | . | . | . | −0.37 | 0.20 | . | * | . | 0.06 | 0.15 |
| Pro | 251 | . | . | B | . | . | . | . | −0.02 | 0.20 | . | * | . | 0.22 | 0.41 |
| Val | 252 | . | . | B | . | . | . | . | 0.08 | −0.37 | . | * | F | 1.28 | 1.53 |
| Lys | 253 | . | . | B | . | . | . | . | −0.07 | −0.51 | . | * | F | 1.74 | 2.35 |
| Thr | 254 | . | . | B | . | . | . | . | 0.30 | −0.33 | . | * | F | 1.60 | 1.25 |
| Lys | 255 | . | . | B | . | . | . | . | 0.29 | −0.37 | . | * | F | 1.44 | 2.26 |
| Pro | 256 | . | . | B | . | . | . | . | −0.31 | −0.40 | . | . | F | 1.28 | 1.12 |
| Leu | 257 | . | A | B | B | . | . | . | 0.30 | 0.29 | . | * | . | 0.02 | 0.64 |
| Ser | 258 | . | A | B | B | . | . | . | −0.60 | 0.56 | . | . | . | −0.44 | 0.50 |
| Met | 259 | . | A | B | B | . | . | . | −0.29 | 1.20 | . | . | . | −0.60 | 0.24 |
| Leu | 260 | . | A | B | B | . | . | . | −0.33 | 0.77 | . | . | . | −0.43 | 0.49 |
| Tyr | 261 | . | . | B | B | . | . | . | −0.47 | 0.49 | . | . | . | −0.26 | 0.58 |
| Val | 262 | . | . | B | . | . | T | . | 0.46 | 0.53 | . | . | . | 0.31 | 0.58 |
| Asp | 263 | . | . | B | . | . | T | . | −0.10 | −0.09 | . | . | F | 1.68 | 1.39 |
| Asn | 264 | . | . | B | . | . | T | . | −0.31 | −0.13 | . | * | F | 1.70 | 0.66 |
| Gly | 265 | A | . | . | . | . | T | . | −0.31 | −0.20 | * | * | F | 1.53 | 0.73 |
| Arg | 266 | A | A | . | . | . | . | . | −0.07 | −0.16 | * | * | F | 0.96 | 0.36 |
| Val | 267 | A | A | . | . | . | . | . | 0.76 | −0.16 | * | * | . | 0.64 | 0.37 |
| Leu | 268 | A | A | . | . | . | . | . | 0.72 | −0.06 | * | * | . | 0.47 | 0.52 |
| Leu | 269 | A | A | . | . | . | . | . | 0.77 | 0.01 | * | * | . | −0.30 | 0.36 |
| Asp | 270 | A | A | . | . | . | . | . | 1.11 | 0.01 | * | * | . | −0.30 | 0.96 |
| His | 271 | A | A | . | . | . | . | . | 0.40 | −0.63 | * | * | . | 0.75 | 1.95 |
| His | 272 | A | A | . | . | . | . | . | 0.37 | −0.70 | . | . | . | 0.75 | 2.34 |
| Lys | 273 | A | A | . | . | . | . | . | 0.32 | −0.70 | * | . | . | 0.60 | 0.98 |
| Asp | 274 | A | A | . | . | . | . | . | 1.13 | −0.06 | . | . | . | 0.30 | 0.54 |
| Met | 275 | A | A | . | . | . | . | . | 1.13 | −0.56 | . | . | . | 0.60 | 0.68 |
| Ile | 276 | A | A | . | . | . | . | . | 0.50 | −1.06 | . | . | . | 0.60 | 0.59 |
| Val | 277 | A | A | . | . | . | . | . | 0.19 | −0.49 | . | . | . | 0.30 | 0.19 |
| Glu | 278 | A | A | . | . | . | . | . | −0.52 | −0.06 | . | . | . | 0.30 | 0.19 |
| Glu | 279 | A | A | . | . | . | . | . | −1.33 | −0.10 | * | . | . | 0.30 | 0.15 |
| Cys | 280 | A | . | . | . | . | T | . | −1.12 | −0.10 | . | . | . | 0.70 | 0.16 |
| Gly | 281 | A | . | . | . | . | T | . | −0.62 | −0.31 | . | . | . | 0.70 | 0.12 |
| Cys | 282 | A | . | . | . | . | T | . | −0.16 | 0.11 | . | . | . | 0.10 | 0.09 |
| Leu | 283 | A | . | . | . | . | T | . | −0.54 | 0.54 | . | . | . | −0.20 | 0.21 |

TABLE II

| Res | Position | I | II | III | IV | V | VI | VII | VIII | IX | X | XI | XII | XIII | XIV |
|---|---|---|---|---|---|---|---|---|---|---|---|---|---|---|---|
| Met | 1 | . | . | B | . | . | . | . | 0.03 | 0.41 | . | . | . | −0.40 | 0.82 |
| Gln | 2 | . | . | B | . | . | T | . | −0.39 | 0.90 | . | . | . | −0.20 | 0.67 |
| Pro | 3 | . | . | B | . | . | T | . | −0.67 | 1.16 | . | . | . | −0.20 | 0.43 |
| Leu | 4 | . | . | . | . | T | T | . | −0.57 | 1.30 | . | . | . | 0.20 | 0.24 |
| Trp | 5 | A | . | . | . | . | T | . | −0.77 | 1.60 | . | . | . | −0.20 | 0.14 |
| Leu | 6 | . | A | B | . | . | . | . | −0.98 | 1.70 | . | . | . | −0.60 | 0.09 |
| Cys | 7 | . | A | B | . | . | . | . | −1.27 | 1.96 | . | . | . | −0.60 | 0.09 |
| Trp | 8 | A | A | . | . | . | . | . | −1.91 | 2.19 | . | . | . | −0.60 | 0.09 |
| Ala | 9 | . | A | B | . | . | . | . | −1.91 | 1.91 | . | . | . | −0.60 | 0.08 |
| Leu | 10 | . | A | B | . | . | . | . | −1.83 | 1.91 | . | . | . | −0.60 | 0.13 |
| Trp | 11 | . | A | B | . | . | . | . | −1.83 | 1.77 | . | . | . | −0.60 | 0.19 |
| Val | 12 | . | A | B | . | . | . | . | −1.76 | 1.54 | . | . | . | −0.60 | 0.16 |
| Leu | 13 | . | A | B | . | . | . | . | −1.77 | 1.54 | . | . | . | −0.60 | 0.19 |
| Pro | 14 | . | . | B | . | . | . | . | −1.39 | 1.24 | . | . | . | −0.40 | 0.24 |
| Leu | 15 | . | . | . | . | T | . | . | −0.92 | 0.76 | . | . | . | 0.00 | 0.50 |
| Ala | 16 | . | . | . | . | . | . | C | −1.22 | 0.54 | . | . | . | −0.20 | 0.61 |
| Ser | 17 | . | . | . | . | . | T | C | −0.96 | 0.36 | . | . | F | 0.45 | 0.40 |
| Pro | 18 | . | . | . | . | . | T | C | −0.96 | 0.43 | . | . | F | 0.15 | 0.48 |
| Gly | 19 | . | . | . | . | . | T | C | −1.06 | 0.43 | . | . | . | 0.00 | 0.40 |
| Ala | 20 | A | . | . | . | . | T | . | −0.59 | 0.41 | . | . | . | −0.20 | 0.43 |
| Ala | 21 | A | A | . | . | . | . | . | −0.00 | 0.46 | . | . | . | −0.60 | 0.27 |
| Leu | 22 | . | A | B | . | . | . | . | 0.30 | 0.03 | . | . | . | −0.30 | 0.48 |
| Thr | 23 | . | A | B | . | . | . | . | −0.30 | 0.00 | . | . | F | −0.15 | 0.82 |
| Gly | 24 | A | A | . | . | . | . | . | −0.77 | 0.19 | . | . | F | −0.15 | 0.67 |
| Glu | 25 | A | A | . | . | . | . | . | −0.52 | 0.37 | . | . | F | −0.15 | 0.67 |
| Gln | 26 | A | A | . | . | . | . | . | −0.23 | 0.11 | . | . | F | −0.15 | 0.46 |
| Leu | 27 | A | A | . | . | . | . | . | −0.23 | 0.01 | . | . | F | −0.15 | 0.62 |
| Leu | 28 | A | A | . | . | . | . | . | −0.73 | 0.27 | * | . | F | −0.15 | 0.30 |
| Gly | 29 | A | A | . | . | . | . | . | −0.28 | 0.96 | * | . | F | −0.45 | 0.14 |
| Ser | 30 | A | A | . | . | . | . | . | −0.28 | 0.56 | * | . | F | −0.45 | 0.33 |
| Leu | 31 | A | A | . | . | . | . | . | −1.09 | 0.27 | * | . | F | −0.30 | 0.70 |
| Leu | 32 | A | A | . | . | . | . | . | −0.28 | 0.27 | * | . | . | −0.30 | 0.58 |
| Arg | 33 | A | A | . | . | . | . | . | −0.28 | 0.24 | * | * | . | −0.30 | 0.76 |
| Gln | 34 | A | A | . | . | . | . | . | 0.11 | 0.54 | . | . | . | −0.60 | 0.76 |
| Leu | 35 | A | A | . | . | . | . | . | 0.41 | −0.14 | . | . | . | 0.45 | 1.83 |
| Gln | 36 | . | A | B | . | . | . | . | 0.37 | −0.83 | . | . | . | 0.75 | 1.62 |
| Leu | 37 | . | A | B | . | . | . | . | 0.97 | −0.19 | . | . | . | 0.30 | 0.69 |
| Lys | 38 | . | A | B | . | . | . | . | 0.54 | −0.16 | . | . | F | 0.60 | 1.30 |
| Glu | 39 | . | A | B | . | . | . | . | −0.27 | −0.36 | . | * | F | 0.60 | 1.08 |
| Val | 40 | . | A | B | . | . | . | . | 0.54 | −0.07 | * | * | F | 0.60 | 1.08 |
| Pro | 41 | . | A | B | . | . | . | . | 0.66 | −0.76 | * | . | F | 0.75 | 0.91 |
| Thr | 42 | A | A | . | . | . | . | . | 0.88 | −0.76 | * | . | F | 0.90 | 1.02 |
| Leu | 43 | A | A | . | . | . | . | . | 0.83 | −0.26 | * | * | F | 0.60 | 1.39 |
| Asp | 44 | A | A | . | . | . | . | . | 0.23 | −0.90 | * | * | F | 0.90 | 1.51 |
| Arg | 45 | A | A | . | . | . | . | . | 1.09 | −0.71 | * | * | F | 0.90 | 1.03 |
| Ala | 46 | A | A | . | . | . | . | . | 1.30 | −1.20 | . | . | F | 0.90 | 2.17 |
| Asp | 47 | A | A | . | . | . | . | . | 0.80 | −1.89 | . | . | . | 0.75 | 2.25 |
| Met | 48 | A | A | . | . | . | . | . | 0.76 | −1.20 | . | . | . | 0.60 | 0.95 |
| Glu | 49 | A | A | . | . | . | . | . | −0.13 | −0.56 | . | * | . | 0.60 | 0.70 |
| Glu | 50 | A | A | . | B | . | . | . | −0.46 | −0.37 | . | * | . | 0.30 | 0.29 |
| Leu | 51 | A | A | . | B | . | . | . | −0.18 | 0.06 | . | . | . | −0.30 | 0.46 |
| Val | 52 | A | A | . | B | . | . | . | −0.21 | −0.07 | . | . | . | 0.30 | 0.38 |
| Ile | 53 | A | A | . | B | . | . | . | −0.47 | 0.43 | . | * | . | −0.60 | 0.30 |
| Pro | 54 | A | A | . | B | . | . | . | −0.36 | 1.07 | . | * | . | −0.60 | 0.27 |
| Thr | 55 | A | . | . | B | . | . | . | −0.94 | 0.39 | . | * | . | −0.30 | 0.71 |
| His | 56 | A | A | . | B | . | . | . | −0.13 | 0.24 | . | * | . | −0.15 | 1.02 |
| Val | 57 | A | A | . | B | . | . | . | 0.48 | −0.04 | . | * | . | 0.45 | 1.14 |
| Arg | 58 | . | A | B | B | . | . | . | 0.51 | 0.29 | . | * | . | −0.15 | 1.24 |
| Ala | 59 | . | A | B | B | . | . | . | 0.13 | 0.44 | . | * | . | −0.60 | 0.68 |
| Gln | 60 | . | A | B | B | . | . | . | −0.37 | 0.44 | . | * | . | −0.60 | 0.92 |
| Tyr | 61 | . | A | B | B | . | . | . | −1.14 | 0.49 | . | * | . | −0.60 | 0.39 |
| Val | 62 | . | A | B | B | . | . | . | −0.29 | 1.17 | . | * | . | −0.60 | 0.32 |
| Ala | 63 | . | A | B | B | . | . | . | −0.29 | 1.07 | . | * | . | −0.60 | 0.32 |
| Leu | 64 | . | A | B | B | . | . | . | −0.00 | 0.67 | * | . | . | −0.60 | 0.40 |
| Leu | 65 | . | A | B | B | . | . | . | −0.03 | 0.30 | * | . | . | 0.04 | 0.72 |
| Gln | 66 | . | A | B | B | . | . | . | −0.13 | 0.16 | * | . | . | 0.38 | 0.96 |
| Arg | 67 | . | A | B | B | . | . | . | 0.72 | 0.09 | . | . | F | 1.02 | 1.16 |
| Ser | 68 | . | A | . | B | T | . | . | 1.42 | −0.60 | . | . | F | 2.66 | 2.34 |
| His | 69 | . | . | . | . | T | T | . | 1.93 | −1.29 | * | * | F | 3.40 | 2.65 |
| Gly | 70 | . | . | . | . | T | T | . | 2.86 | −1.30 | * | * | F | 3.06 | 1.81 |
| Asp | 71 | . | . | . | . | T | T | . | 2.51 | −1.30 | . | * | F | 3.06 | 2.65 |
| Arg | 72 | . | . | . | . | T | T | . | 2.44 | −1.26 | . | . | F | 3.06 | 1.93 |
| Ser | 73 | . | . | . | . | T | T | . | 2.86 | −1.76 | . | . | F | 3.06 | 3.90 |
| Arg | 74 | . | . | . | . | T | T | . | 2.19 | −2.19 | . | . | F | 3.06 | 4.57 |
| Gly | 75 | . | . | . | . | T | T | . | 2.23 | −1.40 | * | * | F | 3.40 | 2.02 |
| Lys | 76 | . | . | . | . | T | T | . | 2.23 | −1.01 | * | * | F | 3.06 | 2.02 |
| Arg | 77 | . | . | . | . | T | . | . | 1.82 | −1.00 | * | * | F | 2.72 | 1.79 |
| Phe | 78 | . | . | B | . | . | . | . | 1.42 | −0.61 | * | * | F | 2.18 | 2.42 |
| Ser | 79 | . | . | B | . | . | T | . | 1.42 | −0.26 | * | * | F | 1.94 | 1.05 |
| Gln | 80 | . | . | B | . | . | T | . | 1.77 | −0.26 | * | * | F | 1.80 | 1.05 |
| Ser | 81 | . | . | B | . | . | T | . | 0.87 | −0.26 | * | * | F | 2.00 | 2.09 |
| Phe | 82 | . | . | B | . | . | T | . | 0.17 | −0.40 | * | * | F | 1.80 | 1.16 |
| Arg | 83 | . | A | B | . | . | . | . | 0.52 | −0.29 | * | * | F | 1.05 | 0.68 |
| Glu | 84 | A | A | . | . | . | . | . | 0.93 | −0.26 | * | * | . | 0.70 | 0.50 |
| Val | 85 | A | A | . | . | . | . | . | 0.23 | −0.64 | * | . | . | 0.95 | 1.13 |
| Ala | 86 | A | A | . | . | . | . | . | −0.28 | −0.64 | * | * | . | 0.60 | 0.50 |
| Gly | 87 | A | A | . | . | . | . | . | −0.17 | 0.04 | * | . | . | −0.30 | 0.24 |
| Arg | 88 | A | A | . | . | . | . | . | −1.09 | 0.54 | * | . | . | −0.60 | 0.32 |
| Phe | 89 | A | A | . | . | . | . | . | −1.09 | 0.59 | * | * | . | −0.60 | 0.26 |
| Leu | 90 | A | A | . | . | . | . | . | −0.82 | 0.09 | . | * | . | −0.30 | 0.46 |
| Ala | 91 | A | A | . | . | . | . | . | −0.53 | 0.16 | * | . | . | −0.30 | 0.24 |
| Leu | 92 | A | A | . | . | . | . | . | −0.50 | 0.54 | . | . | . | −0.60 | 0.37 |
| Glu | 93 | A | A | . | . | . | . | . | −0.64 | 0.24 | . | * | . | −0.30 | 0.65 |
| Ala | 94 | A | A | . | . | . | . | . | −0.76 | 0.06 | . | . | . | −0.30 | 0.87 |
| Ser | 95 | A | . | . | B | . | . | . | −0.76 | 0.24 | . | * | F | −0.15 | 0.87 |
| Thr | 96 | A | . | . | B | . | . | . | −1.02 | 0.24 | . | . | . | −0.30 | 0.41 |
| His | 97 | A | . | . | B | . | . | . | −0.91 | 0.89 | . | * | . | −0.60 | 0.30 |
| Leu | 98 | A | . | . | B | . | . | . | −1.26 | 1.17 | . | . | . | −0.60 | 0.20 |
| Leu | 99 | A | . | . | B | . | . | . | −1.27 | 1.21 | . | . | . | −0.60 | 0.13 |
| Val | 100 | A | . | . | B | . | . | . | −0.97 | 1.34 | . | . | . | −0.60 | 0.10 |
| Phe | 101 | . | . | B | B | . | . | . | −0.66 | 0.84 | . | . | . | −0.60 | 0.21 |
| Gly | 102 | . | . | B | B | . | . | . | −0.51 | 0.56 | . | * | . | −0.60 | 0.43 |
| Met | 103 | . | A | B | . | . | . | . | −0.51 | −0.13 | . | * | . | 0.45 | 1.14 |
| Glu | 104 | . | A | B | . | . | . | . | 0.09 | −0.09 | . | * | F | 0.60 | 1.09 |
| Gln | 105 | . | A | B | . | . | . | . | 0.73 | −0.44 | * | * | F | 0.90 | 1.70 |
| Arg | 106 | . | A | . | . | . | . | C | 1.43 | −0.44 | . | * | F | 1.40 | 2.66 |
| Leu | 107 | . | A | . | . | . | . | C | 1.48 | −0.66 | . | * | F | 2.00 | 2.47 |
| Pro | 108 | . | . | . | . | . | T | C | 2.08 | −0.27 | . | * | F | 2.40 | 1.91 |
| Pro | 109 | . | − | . | . | . | T | C | 1.27 | −0.67 | . | * | F | 3.00 | 1.69 |
| Asn | 110 | . | . | . | . | . | T | C | 0.41 | 0.01 | . | * | F | 1.80 | 1.69 |
| Ser | 111 | . | . | . | . | . | T | C | 0.30 | −0.03 | . | * | F | 1.95 | 0.81 |
| Glu | 112 | A | A | . | . | . | . | . | 0.52 | −0.06 | * | . | F | 1.05 | 0.91 |
| Leu | 113 | A | A | . | . | . | . | . | −0.12 | 0.01 | . | . | . | 0.00 | 0.57 |
| Val | 114 | A | A | . | . | . | . | . | −0.72 | 0.26 | * | * | . | −0.30 | 0.32 |
| Gln | 115 | A | A | . | . | . | . | . | −0.61 | 0.56 | * | * | . | −0.60 | 0.15 |
| Ala | 116 | A | A | . | . | . | . | . | −1.12 | 0.56 | * | * | . | −0.60 | 0.36 |
| Val | 117 | A | A | . | . | . | . | . | −1.82 | 0.56 | * | . | . | −0.60 | 0.40 |
| Leu | 118 | . | A | B | . | . | . | . | −1.01 | 0.70 | * | * | . | −0.60 | 0.20 |
| Arg | 119 | . | A | B | . | . | . | . | −0.16 | 0.70 | * | * | . | −0.60 | 0.34 |
| Leu | 120 | . | A | B | . | . | . | . | −0.37 | 0.20 | * | * | . | −0.30 | 0.79 |
| Phe | 121 | . | A | B | . | . | . | . | −0.63 | −0.01 | * | . | . | 0.45 | 1.49 |
| Gln | 122 | . | A | B | . | . | . | . | 0.01 | −0.06 | * | . | F | 0.45 | 0.56 |
| Glu | 123 | . | A | . | . | . | . | C | 0.87 | 0.37 | * | * | F | 0.20 | 1.06 |
| Pro | 124 | A | A | . | . | . | . | . | 0.17 | −0.31 | * | . | F | 0.60 | 2.44 |
| Val | 125 | A | A | . | . | . | . | . | 0.39 | −0.60 | * | . | F | 0.90 | 1.42 |
| Pro | 126 | A | A | . | . | . | . | . | 0.28 | −0.50 | * | . | F | 0.45 | 0.83 |
| Lys | 127 | A | A | . | . | . | . | . | 0.24 | 0.19 | . | . | F | −0.15 | 0.44 |
| Ala | 128 | A | A | . | . | . | . | . | 0.36 | 0.26 | . | . | . | −0.30 | 0.81 |
| Ala | 129 | A | A | . | . | . | . | . | 0.53 | −0.39 | . | . | . | 0.45 | 1.03 |
| Leu | 130 | A | A | . | . | . | . | . | 1.04 | −0.31 | * | . | . | 0.30 | 0.70 |
| His | 131 | A | . | . | . | . | T | . | 1.37 | 0.11 | * | * | . | 0.10 | 0.69 |
| Arg | 132 | . | . | B | . | . | T | . | 0.51 | −0.39 | * | * | . | 0.85 | 1.33 |
| His | 133 | . | . | . | . | T | T | . | 0.80 | −0.20 | * | * | . | 1.25 | 1.33 |
| Gly | 134 | . | . | . | . | T | T | . | 1.18 | −0.50 | * | * | . | 1.25 | 1.31 |
| Arg | 135 | . | . | . | . | T | . | . | 2.10 | −0.57 | * | * | F | 1.84 | 1.03 |
| Leu | 136 | . | . | . | . | . | . | C | 1.83 | −0.57 | * | * | F | 1.98 | 1.49 |
| Ser | 137 | . | . | . | . | . | T | C | 1.13 | −0.69 | * | * | F | 2.52 | 2.01 |
| Pro | 138 | . | . | . | . | . | T | C | 1.28 | −0.61 | * | * | F | 2.86 | 1.04 |
| Arg | 139 | . | . | . | . | T | T | . | 1.03 | −0.61 | * | * | F | 3.40 | 2.47 |
| Ser | 140 | . | . | . | . | . | T | C | 1.03 | −0.80 | * | * | F | 2.86 | 1.86 |
| Ala | 141 | . | . | B | . | . | . | . | 0.99 | −1.19 | . | * | F | 2.12 | 2.36 |
| Arg | 142 | . | . | B | B | . | . | . | 0.98 | −0.97 | . | * | . | 1.28 | 0.89 |
| Ala | 143 | . | . | B | B | . | . | . | 0.33 | −0.49 | . | * | . | 0.64 | 0.96 |
| Arg | 144 | . | . | B | B | . | . | . | 0.22 | −0.23 | . | * | . | 0.30 | 0.71 |
| Val | 145 | . | . | B | B | . | . | . | 0.23 | −0.73 | . | * | . | 0.60 | 0.62 |
| Thr | 146 | . | . | B | B | . | . | . | 0.01 | 0.19 | * | * | . | −0.30 | 0.65 |
| Val | 147 | . | . | B | B | . | . | . | 0.01 | 0.37 | * | * | . | −0.30 | 0.27 |
| Glu | 148 | . | . | B | B | . | . | . | −0.26 | 0.37 | * | * | . | −0.30 | 0.72 |
| Trp | 149 | . | . | B | B | . | . | . | −0.26 | 0.37 | * | * | . | −0.30 | 0.37 |
| Leu | 150 | . | . | B | B | . | . | . | 0.60 | −0.11 | . | * | . | 0.64 | 0.98 |
| Arg | 151 | . | . | B | B | . | . | . | 0.91 | −0.76 | . | * | . | 1.28 | 0.95 |
| Val | 152 | . | . | B | B | . | . | . | 1.42 | −0.76 | . | * | . | 1.77 | 1.50 |
| Arg | 153 | . | . | . | B | T | . | . | 1.12 | −1.24 | * | * | F | 2.66 | 1.80 |
| Asp | 154 | . | . | . | . | T | T | . | 1.41 | −1.54 | * | * | F | 3.40 | 1.23 |
| Asp | 155 | . | . | . | . | T | T | . | 2.33 | −1.14 | * | * | F | 3.06 | 2.67 |
| Gly | 156 | . | . | . | . | T | T | . | 1.91 | −1.79 | . | * | F | 2.72 | 2.67 |
| Ser | 157 | . | . | . | . | . | T | C | 2.47 | −1.30 | . | * | F | 2.35 | 2.31 |
| Asn | 158 | . | . | . | . | . | T | C | 1.54 | −0.91 | . | * | F | 2.18 | 1.85 |
| Arg | 159 | . | . | B | . | . | T | . | 0.66 | −0.23 | . | . | F | 1.51 | 1.54 |
| Thr | 160 | . | . | B | . | . | T | . | 0.66 | 0.03 | . | . | F | 0.93 | 0.81 |
| Ser | 161 | . | . | B | . | . | T | . | 0.70 | −0.36 | . | * | F | 1.70 | 0.84 |
| Leu | 162 | . | . | B | . | . | . | . | 1.11 | −0.37 | . | * | F | 1.33 | 0.57 |
| Ile | 163 | . | . | B | . | . | . | . | 0.30 | −0.37 | * | . | F | 1.16 | 0.78 |
| Asp | 164 | . | . | B | . | . | T | . | −0.67 | −0.17 | * | * | F | 1.19 | 0.48 |
| Ser | 165 | . | . | B | . | . | T | . | −0.66 | 0.09 | . | . | F | 0.42 | 0.43 |
| Arg | 166 | . | . | B | . | . | T | . | −1.21 | −0.21 | . | . | F | 0.85 | 0.82 |
| Leu | 167 | . | . | B | . | . | T | . | −0.43 | −0.26 | . | . | . | 0.70 | 0.37 |
| Val | 168 | . | . | B | . | . | . | . | 0.46 | 0.24 | * | * | . | −0.10 | 0.37 |
| Ser | 169 | . | . | B | . | . | . | . | 0.16 | −0.14 | . | . | . | 0.50 | 0.33 |
| Val | 170 | . | . | B | . | . | . | . | 0.11 | 0.24 | * | . | . | 0.18 | 0.53 |
| His | 171 | . | . | B | . | . | . | . | −0.29 | −0.01 | * | . | . | 1.06 | 0.71 |
| Glu | 172 | A | . | . | . | . | T | . | 0.57 | 0.26 | * | . | F | 1.09 | 0.56 |
| Ser | 173 | A | . | . | . | . | T | . | 0.83 | −0.13 | * | . | F | 2.12 | 1.51 |
| Gly | 174 | . | . | . | . | T | T | . | 0.43 | −0.27 | * | . | F | 2.80 | 1.12 |
| Trp | 175 | A | . | . | . | . | T | . | 1.29 | 0.01 | * | . | F | 1.37 | 0.56 |
| Lys | 176 | A | A | . | . | . | . | . | 0.47 | 0.01 | * | . | . | 0.54 | 0.70 |
| Ma | 177 | A | A | . | . | . | . | . | 0.16 | 0.27 | * | . | . | 0.26 | 0.52 |
| Phe | 178 | A | A | . | . | . | . | . | 0.46 | 0.33 | . | . | . | −0.02 | 0.72 |
| Asp | 179 | A | A | . | . | . | . | . | 0.21 | −0.59 | * | . | . | 0.60 | 0.62 |
| Val | 180 | A | A | . | . | . | . | . | −0.36 | −0.09 | . | . | . | 0.30 | 0.62 |
| Thr | 181 | A | A | . | . | . | . | . | −0.40 | 0.06 | . | * | . | −0.30 | 0.53 |
| Glu | 182 | A | A | . | . | . | . | . | −0.51 | −0.33 | * | * | . | 0.30 | 0.51 |
| Ala | 183 | A | A | . | . | . | . | . | −0.10 | 0.46 | . | * | . | −0.60 | 0.60 |
| Val | 184 | A | A | . | . | . | . | . | −0.10 | 0.73 | * | . | . | −0.60 | 0.44 |
| Asn | 185 | A | A | . | . | . | . | . | 0.76 | 0.64 | * | . | . | −0.60 | 0.44 |
| Phe | 186 | A | A | . | . | . | . | . | 0.26 | 1.04 | * | . | . | −0.60 | 0.75 |
| Trp | 187 | A | A | . | . | . | . | . | −0.04 | 1.23 | * | . | . | −0.60 | 0.83 |
| Gln | 188 | A | A | . | . | . | . | . | 0.66 | 0.97 | * | . | . | −0.60 | 0.69 |
| Gln | 189 | . | A | . | . | T | . | . | 1.30 | 0.57 | * | * | . | 0.29 | 1.56 |
| Leu | 190 | . | A | . | . | T | . | . | 1.41 | 0.21 | * | * | F | 1.08 | 2.30 |
| Ser | 191 | . | A | . | . | . | . | C | 2.11 | −0.70 | * | . | F | 2.12 | 2.60 |
| Arg | 192 | . | . | . | . | . | T | C | 2.19 | −0.70 | * | * | F | 2.86 | 2.60 |
| Pro | 193 | . | . | . | . | T | T | . | 1.38 | −0.67 | * | . | F | 3.40 | 4.88 |
| Arg | 194 | . | . | . | . | T | T | . | 0.57 | −0.67 | . | * | F | 3.06 | 3.00 |
| Gln | 195 | . | . | B | . | . | T | . | 0.57 | −0.37 | . | * | F | 2.02 | 1.26 |
| Pro | 196 | . | A | B | . | . | . | . | 0.87 | 0.31 | . | * | F | 0.53 | 0.67 |
| Leu | 197 | . | A | B | . | . | . | . | −0.10 | 0.29 | . | * | F | 0.19 | 0.60 |
| Leu | 198 | . | A | B | . | . | . | . | 0.19 | 0.93 | . | * | . | −0.60 | 0.26 |
| Leu | 199 | . | A | B | . | . | . | . | −1.16 | 0.91 | . | * | . | −0.60 | 0.22 |
| Gln | 200 | . | A | B | . | . | . | . | −1.16 | 1.13 | . | . | . | −0.60 | 0.20 |
| Val | 201 | . | A | B | . | . | . | . | −0.83 | 0.84 | . | * | . | −0.60 | 0.42 |
| Ser | 202 | . | . | B | B | . | . | . | −0.02 | 0.16 | . | * | . | −0.30 | 0.99 |
| Val | 203 | . | A | B | B | . | . | . | 0.76 | −0.53 | . | . | . | 0.60 | 0.99 |
| Gln | 204 | . | A | B | B | . | . | . | 0.76 | −0.43 | . | * | F | 0.60 | 1.82 |
| Arg | 205 | . | A | B | B | . | . | . | 0.41 | −0.39 | . | . | F | 0.60 | 1.12 |
| Glu | 206 | . | A | B | B | . | . | . | 1.06 | −0.34 | . | . | F | 0.60 | 1.50 |
| His | 207 | . | A | B | . | . | . | . | 0.54 | −0.56 | . | . | F | 0.90 | 1.34 |
| Leu | 208 | . | A | . | . | . | . | C | 0.81 | −0.27 | . | . | F | 0.65 | 0.56 |
| Gly | 209 | . | A | . | . | . | . | C | 0.51 | 0.23 | . | . | F | 0.05 | 0.33 |
| Pro | 210 | . | . | . | . | . | . | C | 0.06 | 0.61 | * | . | F | −0.05 | 0.32 |
| Leu | 211 | A | . | . | . | . | . | . | −0.53 | 0.54 | * | . | F | −0.25 | 0.39 |
| Ala | 212 | A | . | . | . | . | T | . | −0.53 | 0.36 | * | . | F | 0.25 | 0.40 |
| Ser | 213 | A | . | . | . | . | T | . | 0.32 | 0.43 | * | . | F | −0.05 | 0.35 |
| Gly | 214 | A | . | . | . | . | T | . | −0.14 | −0.00 | * | . | . | 0.70 | 0.84 |
| Ala | 215 | A | . | . | . | . | T | . | −0.79 | −0.00 | * | . | . | 0.70 | 0.69 |
| His | 216 | A | A | . | . | . | . | . | 0.13 | 0.14 | * | . | . | −0.30 | 0.38 |
| Lys | 217 | A | A | . | . | . | . | . | 0.02 | −0.24 | * | . | . | 0.30 | 0.76 |
| Leu | 218 | . | A | B | . | . | . | . | −0.27 | 0.11 | * | . | . | −0.30 | 0.65 |
| Val | 219 | . | A | B | . | . | . | . | −0.22 | 0.11 | * | . | . | −0.30 | 0.48 |
| Arg | 220 | . | A | B | . | . | . | . | 0.37 | −0.00 | * | * | . | 0.30 | 0.32 |
| Phe | 221 | . | A | B | . | . | . | . | 0.06 | 0.40 | * | . | . | −0.30 | 0.68 |
| Ala | 222 | . | A | B | . | . | . | . | −0.58 | 0.14 | * | . | . | −0.30 | 0.90 |
| Ser | 223 | . | . | . | . | . | T | C | 0.02 | −0.00 | * | * | F | 1.05 | 0.47 |
| Gln | 224 | . | . | . | . | T | T | . | 0.29 | 0.43 | * | * | F | 0.35 | 0.83 |
| Gly | 225 | . | . | . | . | . | T | C | −0.17 | 0.14 | * | * | F | 0.45 | 0.83 |
| Ala | 226 | . | . | . | . | . | T | C | −0.28 | 0.07 | . | . | F | 0.66 | 0.61 |
| Pro | 227 | . | . | . | . | . | T | C | −0.03 | 0.37 | . | . | F | 0.87 | 0.29 |
| Ala | 228 | . | . | . | . | . | T | C | 0.27 | 0.40 | . | . | . | 0.93 | 0.29 |
| Gly | 229 | . | . | . | . | . | T | C | 0.06 | −0.03 | . | . | . | 1.74 | 0.50 |
| Leu | 230 | . | . | . | . | . | T | C | 0.40 | −0.10 | . | . | F | 2.10 | 0.50 |
| Gly | 231 | . | . | . | . | . | . | C | 0.18 | −0.13 | . | * | F | 1.69 | 0.86 |
| Glu | 232 | . | A | . | . | . | . | C | 0.39 | 0.06 | . | * | F | 0.68 | 0.72 |
| Pro | 233 | A | A | . | . | . | . | . | 0.17 | −0.37 | . | * | F | 1.02 | 1.50 |
| Gln | 234 | A | A | . | . | . | . | . | 0.48 | −0.37 | . | * | F | 0.81 | 1.25 |
| Leu | 235 | A | A | . | . | . | . | . | 0.98 | −0.30 | . | * | . | 0.30 | 0.98 |
| Glu | 236 | A | A | . | . | . | . | . | 0.51 | 0.19 | . | * | . | −0.30 | 0.92 |
| Leu | 237 | A | A | . | . | . | . | . | 0.51 | 0.44 | . | * | . | −0.60 | 0.44 |
| His | 238 | A | A | . | . | . | . | . | −0.09 | 0.04 | . | * | . | −0.30 | 0.89 |
| Thr | 239 | A | A | . | . | . | . | . | −0.43 | 0.04 | . | . | . | −0.30 | 0.42 |
| Leu | 240 | . | A | B | . | . | . | . | 0.38 | 0.47 | . | . | . | −0.60 | 0.51 |
| Asp | 241 | . | A | B | . | . | . | . | 0.13 | −0.21 | . | . | . | 0.30 | 0.62 |
| Leu | 242 | . | A | B | . | . | . | . | 0.60 | 0.04 | . | . | . | −0.30 | 0.67 |
| Gly | 243 | . | . | . | . | T | T | . | 0.04 | −0.01 | * | . | F | 1.25 | 0.81 |
| Asp | 244 | . | . | . | . | T | T | . | 0.36 | −0.20 | * | . | F | 1.25 | 0.49 |
| Tyr | 245 | . | . | . | . | T | T | . | 0.82 | 0.20 | . | * | F | 1.11 | 1.03 |
| Gly | 246 | . | . | . | . | T | T | . | 0.82 | −0.06 | * | * | F | 2.02 | 1.03 |
| Ala | 247 | . | . | . | . | T | . | . | 0.97 | −0.49 | . | * | F | 2.13 | 1.03 |
| Gln | 248 | . | . | B | . | . | T | . | 1.31 | 0.09 | . | * | F | 1.49 | 0.35 |
| Gly | 249 | . | . | . | . | T | T | . | 1.10 | −0.67 | . | * | F | 3.10 | 0.59 |
| Asp | 250 | . | . | . | . | T | T | . | 1.34 | −0.67 | . | * | F | 2.79 | 0.91 |
| Cys | 251 | . | . | . | . | . | T | C | 1.10 | −1.17 | . | * | F | 2.28 | 0.91 |
| Asp | 252 | . | . | . | . | . | . | C | 1.48 | −1.07 | . | * | F | 1.77 | 0.93 |
| Pro | 253 | . | . | . | . | . | . | C | 0.88 | −1.07 | . | * | F | 1.46 | 0.86 |
| Glu | 254 | . | A | . | . | . | . | C | 0.91 | −0.46 | . | * | F | 0.80 | 1.58 |
| Ala | 255 | A | A | . | . | . | . | . | 0.91 | −0.54 | . | * | F | 0.90 | 1.37 |
| Pro | 256 | A | A | . | . | . | . | . | 1.23 | −0.54 | . | . | F | 0.90 | 1.53 |
| Met | 257 | A | A | . | . | . | . | . | 0.92 | −0.54 | * | . | F | 0.75 | 0.88 |
| Thr | 258 | A | A | . | . | . | . | . | 1.24 | −0.06 | * | . | F | 0.60 | 1.25 |
| Glu | 259 | A | A | . | . | . | . | . | 0.58 | −0.56 | * | . | F | 0.90 | 1.59 |
| Gly | 260 | . | . | . | . | T | T | . | 0.50 | −0.41 | * | . | F | 1.25 | 0.86 |
| Thr | 261 | A | . | . | . | . | T | . | 0.82 | −0.46 | * | . | F | 0.85 | 0.32 |
| Arg | 262 | A | . | . | . | . | T | . | 1.42 | −0.94 | * | . | F | 1.15 | 0.36 |
| Cys | 263 | A | . | . | . | . | T | . | 1.73 | −0.54 | * | . | . | 1.00 | 0.63 |
| Cys | 264 | A | A | . | . | . | . | . | 1.13 | −0.97 | * | . | . | 0.60 | 0.76 |
| Arg | 265 | A | A | . | . | . | . | . | 1.23 | −0.84 | * | . | . | 0.60 | 0.38 |
| Gln | 266 | . | A | B | . | . | . | . | 0.66 | −0.09 | * | * | F | 0.60 | 1.12 |
| Glu | 267 | . | A | B | . | . | . | . | 0.54 | 0.03 | . | * | . | −0.15 | 1.46 |
| Met | 268 | . | A | B | . | . | . | . | 0.40 | −0.54 | . | * | . | 0.75 | 1.25 |
| Tyr | 269 | . | A | B | . | . | . | . | 1.07 | 0.14 | . | * | . | −0.30 | 0.59 |
| Ile | 270 | A | A | . | . | . | . | . | 0.61 | 0.14 | . | * | . | −0.30 | 0.59 |
| Asp | 271 | A | A | . | . | . | . | . | 0.01 | 0.57 | . | * | . | −0.60 | 0.59 |
| Leu | 272 | A | A | . | . | . | . | . | 0.06 | 0.57 | . | * | . | −0.60 | 0.38 |
| Gln | 273 | A | A | . | . | . | . | . | 0.37 | −0.19 | . | * | . | 0.45 | 1.07 |
| Gly | 274 | A | A | . | . | . | . | . | 0.02 | 0.04 | . | . | . | −0.30 | 0.67 |
| Met | 275 | A | A | . | . | . | . | . | 0.91 | 0.54 | * | * | . | −0.60 | 0.83 |
| Lys | 276 | A | A | . | . | . | . | . | 0.91 | −0.14 | * | . | . | 0.30 | 0.83 |
| Trp | 277 | A | A | . | . | . | . | . | 1.43 | −0.14 | * | . | . | 0.45 | 1.34 |
| Ala | 278 | A | A | . | . | . | . | . | 0.58 | 0.34 | * | . | . | −0.15 | 1.43 |
| Glu | 279 | A | A | . | . | . | . | . | 0.11 | 0.37 | * | . | . | −0.30 | 0.53 |
| Asn | 280 | A | A | . | . | . | . | . | 0.71 | 1.06 | * | * | . | −0.60 | 0.42 |
| Trp | 281 | . | A | B | . | . | . | . | 0.46 | 0.14 | * | . | . | −0.30 | 0.71 |
| Val | 282 | . | A | . | . | . | . | C | 0.53 | 0.07 | . | . | . | −0.10 | 0.64 |
| Leu | 283 | . | A | . | . | . | . | C | 0.78 | 0.50 | . | . | . | −0.40 | 0.61 |
| Glu | 284 | . | A | . | . | . | . | C | 0.08 | 0.53 | . | . | F | −0.25 | 0.57 |
| Pro | 285 | . | . | . | . | . | T | C | −0.73 | 0.40 | . | . | F | 0.45 | 0.67 |
| Pro | 286 | . | . | . | . | T | T | . | −1.03 | 0.44 | . | . | F | 0.35 | 0.67 |
| Gly | 287 | . | . | . | . | T | T | . | −0.42 | 0.26 | . | . | . | 0.50 | 0.39 |
| Phe | 288 | A | . | . | . | . | T | . | 0.39 | 1.01 | . | . | . | −0.20 | 0.40 |
| Leu | 289 | A | A | . | . | . | . | . | −0.28 | 0.59 | . | . | . | −0.60 | 0.44 |
| Ala | 290 | A | A | B | . | . | . | . | −0.92 | 0.73 | . | . | . | −0.60 | 0.24 |
| Tyr | 291 | . | A | B | . | . | . | . | −1.06 | 0.94 | . | . | . | −0.60 | 0.21 |
| Glu | 292 | . | A | B | . | . | . | . | −1.02 | 0.59 | . | . | . | −0.60 | 0.25 |
| Cys | 293 | . | A | . | . | T | . | . | −0.99 | 0.39 | . | * | . | 0.10 | 0.35 |
| Val | 294 | . | . | . | . | T | . | . | −0.07 | 0.46 | * | . | . | 0.00 | 0.12 |
| Gly | 295 | . | . | . | . | T | T | . | 0.52 | −0.30 | . | . | . | 1.10 | 0.14 |
| Thr | 296 | . | . | . | . | T | T | . | 0.56 | 0.10 | . | . | F | 0.95 | 0.44 |
| Cys | 297 | . | . | . | . | T | T | . | 0.34 | −0.04 | * | . | F | 1.85 | 0.92 |
| Arg | 298 | . | . | . | . | T | T | . | 1.01 | −0.26 | * | . | F | 2.30 | 1.44 |
| Gln | 299 | . | . | . | . | . | . | C | 1.28 | −0.69 | * | . | F | 2.50 | 1.73 |
| Pro | 300 | . | . | . | . | . | T | C | 0.81 | −0.67 | * | . | F | 3.00 | 3.25 |
| Pro | 301 | . | . | . | . | . | T | C | 0.53 | −0.56 | * | . | F | 2.70 | 1.37 |
| Glu | 302 | A | . | . | . | . | T | . | 0.50 | −0.06 | . | * | F | 1.75 | 0.80 |
| Ala | 303 | A | . | . | . | . | T | . | 0.43 | 0.33 | . | * | . | 0.70 | 0.45 |
| Leu | 304 | A | A | . | . | . | . | . | 0.14 | −0.10 | . | . | . | 0.60 | 0.58 |
| Ala | 305 | A | A | . | . | . | . | . | 0.14 | 0.39 | . | * | . | −0.30 | 0.35 |
| Phe | 306 | A | A | . | . | . | . | . | −0.34 | 0.81 | . | . | . | −0.60 | 0.54 |
| Lys | 307 | A | A | . | . | . | . | . | −1.16 | 1.10 | . | * | . | −0.60 | 0.56 |
| Trp | 308 | . | A | B | . | . | . | . | −0.91 | 1.10 | . | * | . | −0.60 | 0.46 |
| Pro | 309 | . | A | . | . | . | . | C | −0.31 | 1.03 | * | * | . | −0.40 | 0.53 |
| Phe | 310 | . | . | . | . | T | . | . | 0.39 | 0.67 | * | . | . | 0.00 | 0.41 |
| Leu | 311 | . | . | . | . | . | . | C | 1.09 | 0.67 | * | * | . | −0.20 | 0.76 |
| Gly | 312 | . | . | . | . | . | T | C | 0.38 | 0.16 | * | * | F | 0.45 | 0.85 |
| Pro | 313 | . | . | . | . | T | T | . | −0.22 | 0.30 | . | . | F | 0.65 | 0.53 |
| Arg | 314 | . | . | . | . | T | T | . | −0.60 | 0.20 | . | . | F | 0.65 | 0.45 |
| Gln | 315 | . | . | . | . | T | T | . | −0.20 | 0.01 | . | . | . | 0.50 | 0.46 |
| Cys | 316 | . | . | B | B | . | . | . | 0.61 | −0.03 | . | . | . | 0.30 | 0.40 |
| Ile | 317 | . | . | B | B | . | . | . | 0.64 | −0.46 | . | . | . | 0.64 | 0.35 |
| Ala | 318 | . | . | B | B | . | . | . | 0.86 | 0.03 | . | . | . | 0.38 | 0.29 |
| Ser | 319 | . | . | B | B | . | . | . | 0.44 | −0.37 | * | . | F | 1.47 | 0.91 |
| Glu | 320 | . | . | B | . | . | T | . | −0.37 | −0.56 | * | . | F | 2.66 | 1.74 |
| Thr | 321 | . | . | . | . | T | T | . | 0.09 | −0.56 | . | . | F | 3.40 | 1.42 |
| Asp | 322 | . | . | . | . | T | T | . | 0.38 | −0.63 | . | . | F | 3.06 | 1.64 |
| Ser | 323 | A | . | . | . | . | T | . | 0.08 | −0.40 | . | . | F | 1.87 | 0.93 |
| Leu | 324 | A | . | . | B | . | . | . | −0.48 | 0.29 | . | . | . | 0.38 | 0.45 |
| Pro | 325 | A | . | . | B | . | . | . | −0.78 | 0.44 | . | . | . | −0.26 | 0.20 |
| Met | 326 | A | . | . | B | . | . | . | −1.36 | 0.83 | . | * | . | −0.60 | 0.20 |
| Ile | 327 | . | . | B | B | . | . | . | −1.31 | 1.13 | . | . | . | −0.60 | 0.17 |
| Val | 328 | . | . | B | B | . | . | . | −1.01 | 0.44 | . | * | . | −0.60 | 0.22 |
| Ser | 329 | . | . | B | . | . | . | . | −0.54 | 0.01 | . | * | . | 0.24 | 0.39 |
| Ile | 330 | . | . | B | . | . | . | . | −0.68 | −0.17 | * | * | F | 1.33 | 0.55 |
| Lys | 331 | . | . | B | . | . | T | . | 0.03 | −0.43 | * | * | F | 1.87 | 0.73 |
| Glu | 332 | . | . | . | . | T | T | . | 0.61 | −1.07 | . | * | F | 3.06 | 1.07 |
| Gly | 333 | . | . | . | . | T | T | . | 1.58 | −0.97 | . | * | F | 3.40 | 2.20 |
| Gly | 334 | . | . | . | . | T | T | . | 1.67 | −1.66 | * | * | F | 3.06 | 2.15 |
| Arg | 335 | . | . | . | . | T | . | . | 2.56 | −1.23 | * | * | F | 2.52 | 1.92 |
| Thr | 336 | . | . | . | . | . | . | C | 1.66 | −0.83 | * | * | F | 1.98 | 3.36 |
| Arg | 337 | . | . | B | B | . | . | . | 0.80 | −0.61 | * | * | F | 1.24 | 2.52 |
| Pro | 338 | . | . | B | B | . | . | . | 0.84 | −0.40 | . | * | F | 0.45 | 0.96 |
| Gln | 339 | . | . | B | B | . | . | . | 0.38 | −0.01 | . | * | . | 0.30 | 0.89 |
| Val | 340 | . | . | B | B | . | . | . | 0.06 | 0.19 | . | * | . | −0.30 | 0.37 |
| Val | 341 | . | . | B | B | . | . | . | 0.37 | 0.61 | . | . | . | −0.60 | 0.37 |
| Ser | 342 | . | . | B | . | . | . | . | −0.34 | 0.59 | . | * | . | −0.40 | 0.35 |
| Leu | 343 | . | . | B | . | . | T | . | −0.02 | 0.80 | . | * | . | −0.20 | 0.46 |
| Pro | 344 | . | . | B | . | . | T | . | −0.88 | 0.16 | . | * | . | 0.25 | 1.22 |
| Asn | 345 | . | . | . | . | T | T | . | −0.02 | 0.16 | . | * | . | 0.50 | 0.68 |
| Met | 346 | A | . | . | . | . | T | . | 0.88 | 0.17 | . | . | . | 0.25 | 1.42 |
| Arg | 347 | A | . | . | . | . | . | . | 0.51 | −0.51 | . | * | . | 0.95 | 1.84 |
| Val | 348 | . | . | B | . | . | . | . | 1.02 | −0.37 | . | * | . | 0.50 | 0.61 |
| Gln | 349 | . | . | B | . | . | T | . | 0.57 | −0.39 | . | * | . | 0.70 | 0.83 |
| Lys | 350 | . | . | B | . | . | T | . | −0.02 | −0.43 | . | * | . | 0.70 | 0.23 |
| Cys | 351 | . | . | B | . | . | T | . | 0.28 | 0.07 | . | * | . | 0.10 | 0.31 |
| Ser | 352 | . | . | B | . | . | T | . | 0.17 | −0.19 | . | * | . | 0.70 | 0.24 |
| Cys | 353 | . | . | B | . | . | . | . | 0.68 | −0.59 | . | . | . | 0.80 | 0.20 |
| Ala | 354 | . | . | B | . | . | T | . | 0.09 | −0.16 | . | . | . | 0.70 | 0.37 |
| Ser | 355 | . | . | . | . | T | T | . | −0.77 | −0.23 | . | . | . | 1.10 | 0.28 |
| Asp | 356 | . | . | . | . | T | T | . | −0.96 | 0.07 | . | . | . | 0.50 | 0.43 |
| Gly | 357 | . | . | . | . | T | T | . | −0.87 | 0.14 | . | . | . | 0.50 | 0.31 |
| Ala | 358 | . | . | B | . | . | . | . | −0.09 | 0.07 | * | . | . | 0.06 | 0.36 |
| Leu | 359 | . | . | B | . | . | . | . | 0.61 | −0.31 | * | . | . | 0.82 | 0.42 |
| Val | 360 | . | . | B | . | . | . | . | 0.10 | −0.31 | * | . | . | 0.98 | 0.84 |
| Pro | 361 | . | . | B | . | . | . | . | 0.10 | −0.06 | * | . | F | 1.29 | 0.69 |
| Arg | 362 | . | . | B | . | . | . | . | 0.23 | −0.16 | * | . | F | 1.60 | 1.44 |
| Arg | 363 | . | . | B | . | . | . | . | 0.43 | −0.41 | * | . | F | 1.44 | 3.00 |
| Leu | 364 | . | . | B | . | . | . | . | 0.86 | −0.63 | * | . | . | 1.43 | 2.48 |
| Gln | 365 | . | . | B | . | . | . | . | 1.32 | −0.63 | * | . | . | 1.27 | 1.62 |
| Pro | 366 | . | . | B | . | . | . | . | 1.14 | −0.20 | * | . | . | 0.81 | 1.06 |

Among highly preferred fragments in this regard are those that comprise regions of Human Nodal or Human Lefty that combine several structural features, such as, two, three, four, five or more of the features set out above.

In another embodiment, the invention provides isolated nucleic acid molecules comprising polynucleotides which hybridize under stringent hybridization conditions to a portion of the polynucleotide in a nucleic acid molecule of the inventions described above, for instance, the cDNA clones contained in ATCC Deposit Nos. 209092, 209135, and 209091 and/or a polynucleotide fragment described above. By “stringent hybridization conditions” is intended overnight incubation at 42° C. in a solution comprising: 50% formamide, 5×SSC (750 mM NaCl, 75 mM trisodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65° C.

Further specific embodiments are directed to polynucleotides corresponding to nucleotides 1-125, 1-90, 1-60, 1-30, 30-125, 30-90, 30-60, 60-125, 60-90, 90-125, 310-930, 350-930, 400-930, 450-930, 500-930, 550-930, 600-930, 650-930, 700-930, 750-930, 800-930, 850-930, 900-930, 310-900, 350-900, 400-900, 450-900, 500-900, 550-900, 600-900, 650-900, 700-900, 750-900, 800-900, 850-900, 310-850, 350-850, 400-850, 450-850, 500-850, 550-850, 600-850, 650-850, 700-850, 750-850, 800-850, 310-800, 350-800, 400-800, 450-800, 500-800, 550-800, 600-800, 650-800, 700-800, 750-800, 310-750, 350-750, 400-750, 450-750, 500-750, 550-750, 600-750, 650-750, 700-750, 310-700, 350-700, 400-700, 450-700, 500-700, 550-700, 600-700, 650-700, 310-650, 350-650, 400-650, 450-650, 500-650, 550-650, 600-650, 310-600, 350-600, 400-600, 450-600, 500-600, 550-600, 310-500, 350-500, 400-500, 450-500, 310-450, 350-450, 400-450, 310-400, 350,-400, 310-350, 1050-1596, 1100-1596, 1150-1596, 1200-1596, 1250-1596, 1300-1596, 1350-1596, 1400-1596, 1450-1596, 1500-1596, 1550-1596, 1050-1550, 1100-1550, 1150-1550, 1200-1550, 1250-1550, 1300-1550, 1350-1550, 1400-1550, 1450-1550, 1500-1550, 1050-1500, 1100-1500, 1150-1500, 1200-1500, 1250-1500, 1300-1500, 1350-1500, 1400-1500, 1450-1500, 1050-1450, 1100-1450, 1150-1450, 1200-1450, 1250-1450, 1300-1450, 1350-1450, 1400-1450, 1050-1400, 1100-1400, 1150-1400, 1200-1400, 1250-1400, 1300-1400, 1350-1400, 1050-1350, 1100-1350, 1150-1350, 1200-1350, 1250-1350, 1300-1350, 1050-1300, 1100-1300, 1150-1300, 1200-1300, 1250-1300, 1050-1250, 1100-1250, 1150-1250, 1200-1250, 1050-1200, 1100-1200, 1150-1200, 1050-1150, 1100-1150, and 1050-1100 of SEQ ID NO:3.

By a polynucleotide which hybridizes to a “portion” of a polynucleotide is intended a polynucleotide (either DNA or RNA) hybridizing to at least about 15 nucleotides (nt), and more preferably at least about 20 nt, still more preferably at least about 30 nt, and even more preferably about 30-70 (e.g., 50) nt of the reference polynucleotide. These are useful as diagnostic probes and primers as discussed above and in more detail below.

By a portion of a polynucleotide of “at least 20 nt in length,” for example, is intended 20 or more contiguous nucleotides from the nucleotide sequence of the reference polynucleotides (e.g., the deposited cDNAs or the nucleotide sequences as shown in FIGS. 1A and B and 2A and B (SEQ ID NO:1 and SEQ ID NO:3, respectively)). Of course, a polynucleotide which hybridizes only to a poly A sequence (such as the 3′ terminal poly(A) tract of the Nodal and Lefty cDNAs shown in FIGS. 1A and B and 2A and B (SEQ ID NO:1 and SEQ ID NO:3, respectively)), or to a complementary stretch of T (or U) residues, would not be included in a polynucleotide of the invention used to hybridize to a portion of a nucleic acid of the invention, since such a polynucleotide would hybridize to any nucleic acid molecule containing a poly (A) stretch or the complement thereof (e.g., practically any double-stranded cDNA clone generated using oligo dT as a primer).

In preferred embodiments, polynucleotides which hybridize to the reference polynucleotides disclosed herein encode polypeptides which either retain substantially the same biological function or activity as the mature form or TGF-β-like active form of the Nodal polypeptide encoded by the polynucleotide sequences depicted in FIGS. 1A and 1B (SEQ ID NO:1) and/or substantially the same biological function or activity as the mature form or TGF-β-like active forms of the Lefty polypeptide encoded by the polynucleotide sequences depicted in FIGS. 2A and 2B (SEQ ID NO:1) depicted in FIGS. 2A and 2B (SEQ ID NO:3), or the cDNAs contained in the deposit (HTLFA20, HNGEF08, and HUKEJ46).

Alternative embodiments are directed to polynucleotides which hybridize to the reference polynucleotide (i.e., a polynucleotide sequence disclosed herein), but do not retain biological activity. While these polynucleotides do not retain biological activity, they have uses, such as, for example, as probes for the polynucleotides of SEQ ID NO:1 or SEQ ID NO:3, for recovery of the polynucleotides, as diagnostic probes, and as PCR primers.

As indicated, nucleic acid molecules of the present invention which encode a Lefty polypeptide may include, but are not limited to those encoding the amino acid sequence of the mature form of the polypeptide, by itself; and the coding sequence for the mature form of the polypeptide and additional sequences, such as those encoding the about 18 amino acid leader or secretory sequence, such as a pre-, or pro- or prepro-protein sequence; the coding sequence of the mature polypeptide, with or without the aforementioned additional coding sequences.

As indicated, nucleic acid molecules of the present invention which encode a Nodal polypeptide may include, but are not limited to, those encoding the amino acid sequence of the complete polypeptide, by itself; and the coding sequence for the complete polypeptide and additional sequences, such as those encoding an added secretory leader sequence, such as a pre-, or pro- or prepro-protein sequence.

Also encoded by nucleic acids of the invention are the above protein sequences together with additional, non-coding sequences, including for example, but not limited to introns and non-coding 5′ and 3′ sequences, such as the transcribed, non-translated sequences that play a role in transcription, mRNA processing, including splicing and polyadenylation signals, for example—ribosome binding and stability of mRNA; an additional coding sequence which codes for additional amino acids, such as those which provide additional functionalities.

Thus, the sequences encoding the polypeptides may be fused to a marker sequence, such as a sequence encoding a peptide which facilitates purification of the fused polypeptide. In certain preferred embodiments of the invention, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described by Gentz and colleagues (Proc. Natl. Acad. Sci. USA 86:821-824 (1989)), for instance, hexa-histidine provides for convenient purification of the fusion protein. The “HA” tag is another peptide useful for purification which corresponds to an epitope derived from the influenza hemagglutinin protein, which has been described by Wilson and coworkers (Cell 37:767 (1984)). As discussed below, other such fusion proteins include the Nodal and Lefty fused to Fc at the N- or C-terminus.

The present invention further relates to variants of the nucleic acid molecules of the present invention, which encode portions, analogs or derivatives of the Nodal and Lefty proteins. Variants may occur naturally, such as a natural allelic variant. By an “allelic variant” is intended one of several alternate forms of a gene occupying a given locus on a chromosome of an organism (Genes II, Lewin, B., ed., John Wiley & Sons, New York (1985)). Non-naturally occurring variants may be produced using art-known mutagenesis techniques.

Such variants include those produced by nucleotide substitutions, deletions or additions. The substitutions, deletions or additions may involve one or more nucleotides. The variants may be altered in coding regions, non-coding regions, or both. Alterations in the coding regions may produce conservative or non-conservative amino acid substitutions, deletions or additions. Especially preferred among these are silent substitutions, additions and deletions, which do not alter the properties and activities of the Nodal and Lefty proteins or portions thereof. Also especially preferred in this regard are conservative substitutions.

Most highly preferred are nucleic acid molecules encoding the mature form of the protein having the amino acid sequence shown in SEQ ID NO:4 or the mature Lefty amino acid sequence encoded by the deposited cDNA clone.

Most highly preferred are nucleic acid molecules encoding the active domain of the proteins having the amino acid sequence shown in SEQ ID NO:2 or SEQ ID NO:4 or the active domains of the Nodal and Lefty amino acid sequences encoded by the deposited cDNA clones. By “active domain”, is meant the C-terminal region of a Nodal or Lefty polypeptide, or fragment thereof, which has been processed either in vitro or in vivo such that the C-terminal region has been cleaved from the remainder of the molecule just C-terminal to one or more of the TGF-β cleavage consensus sites as indicated in FIGS. 1A and 1B and 2A and 2B.

Further embodiments include an isolated nucleic acid molecule comprising a polynucleotide having a nucleotide sequence at least 90% identical, and more preferably at least 95%, 96%, 97%, 98% or 99% identical to a polynucleotide selected from the group consisting of: (a) a nucleotide sequence encoding the Nodal polypeptide having the complete amino acid sequence in SEQ ID NO:2 (i.e., positions 1 to 283 of SEQ ID NO:2); (b) a nucleotide sequence encoding the predicted active Nodal polypeptide having the amino acid sequence at positions 173 to 283 of SEQ ID NO:2; (c) a nucleotide sequence encoding the Nodal polypeptide having the complete amino acid sequence encoded by the cDNA clone contained in ATCC Deposit No. 209092 and/or 209135; (d) a nucleotide sequence encoding the active domain of the Nodal polypeptide having the amino acid sequence encoded by the cDNA clone contained in ATCC Deposit No. 209092 and/or 209135; (e) a nucleotide sequence encoding the Lefty polypeptide having the complete amino acid sequence in SEQ ID NO:4 (i.e., positions −18 to 348 of SEQ ID NO:4); (f) a nucleotide sequence encoding the Lefty polypeptide having the complete amino acid sequence in SEQ ID NO:4 excepting the N-terminal methionine (i.e., positions −17 to 348 of SEQ ID NO:4); (g) a nucleotide sequence encoding the predicted active domain of the Lefty polypeptide having the amino acid sequence at positions 60 to 348 of SEQ ID NO:4; (h) a nucleotide sequence encoding the predicted active domain of the Lefty polypeptide having the amino acid sequence at positions 118 to 348 of SEQ ID NO:4; (i) a nucleotide sequence encoding the predicted active domain of the Lefty polypeptide having the amino acid sequence at positions 125 to 348 of SEQ ID NO:4; (j) a nucleotide sequence encoding the Lefty polypeptide having the complete amino acid sequence encoded by the cDNA clone contained in ATCC Deposit No. 209091; (k) a nucleotide sequence encoding the Lefty polypeptide having the complete amino acid sequence excepting the N-terminal methionine encoded by the cDNA clone contained in ATCC Deposit No. 209091; (l) a nucleotide sequence encoding the active domain of the Lefty polypeptide having the amino acid sequence encoded by the cDNA clone contained in ATCC Deposit No. 209091; and (m) a nucleotide sequence complementary to any of the nucleotide sequences in (a) through (l) above.

Further embodiments of the invention include isolated nucleic acid molecules that comprise a polynucleotide having a nucleotide sequence at least 90% identical, and more preferably at least 95%, 96%, 97%, 98% or 99% identical, to any of the nucleotide sequences in (a) through (m) above, or a polynucleotide which hybridizes under stringent hybridization conditions to a polynucleotide in (a) through (m) above. This polynucleotide which hybridizes does not hybridize under stringent hybridization conditions to a polynucleotide having a nucleotide sequence consisting of only A residues or of only T residues. An additional nucleic acid embodiment of the invention relates to an isolated nucleic acid molecule comprising a polynucleotide which encodes the amino acid sequence of an epitope-bearing portion of a Nodal and Lefty polypeptide having an amino acid sequence in (a) through (l) above. A further nucleic acid embodiment of the invention relates to an isolated nucleic acid molecule comprising a polynucleotide which encodes the amino acid sequence of a Human Nodal or Human Lefty polypeptide having an amino acid sequence which contains at least one conservative amino acid substitution, but not more than 50 conservative amino acid substitutions, even more preferably, not more than 40 conservative amino acid substitutions, still more preferably not more than 30 conservative amino acid substitutions, and still even more preferably not more than 20 conservative amino acid substitutions. Of course, in order of ever-increasing preference, it is highly preferable for a polynucleotide which encodes the amino acid sequence of a Human Nodal or Human Lefty polypeptide to have an amino acid sequence which contains not more than 7-10, 5-10, 3-7, 3-5, 2-5, 1-5, 1-3, 10, 9, 8, 7, 6, 5, 4, 3, 2 or 1 conservative amino acid substitutions.

By a polynucleotide having a nucleotide sequence at least, for example, 95% “identical” to a reference nucleotide sequence encoding a Nodal or Lefty polypeptide is intended that the nucleotide sequence of the polynucleotide is identical to the reference sequence except that the polynucleotide sequence may include up to five point mutations per each 100 nucleotides of the reference nucleotide sequences encoding the Nodal and Lefty polypeptides. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 90%, 95%, 96%, 97%, 98% or 99% identical to, for instance, the nucleotide sequences shown in FIGS. 1A and B and 2A and B or to the nucleotides sequence of the deposited cDNA clones can be determined conventionally using known computer programs such as the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman to find the best segment of homology between two sequences (Advances in Applied Mathematics 2:482-489 (1981)). When using Bestfit or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed. A preferred method for determining the best overall match between a query sequence (a sequence of the present invention) and a subject sequence, also referred to as a global sequence alignment, can be determined using the FASTDB computer program based on the algorithm of Brutlag and colleagues (Comp. App. Biosci. 6:237-245 (1990)). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500 or the length of the subject nucleotide sequence, whichever is shorter.

If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for. No other manual corrections are to made for the purposes of the present invention.

The present application is directed to nucleic acid molecules at least 90%, 95%, 96%, 97%, 98% or 99% identical to the nucleic acid sequences shown in FIGS. 1A and B and 2A and B (SEQ ID NO:1 and SEQ ID NO:3, respectively) or to the nucleic acid sequences of the deposited cDNAs, irrespective of whether they encode a polypeptide having Nodal or Lefty activity. This is because even where a particular nucleic acid molecule does not encode a polypeptide having Nodal or Lefty activity, one of skill in the art would still know how to use the nucleic acid molecule, for instance, as a hybridization probe or a polymerase chain reaction (PCR) primer. Uses of the nucleic acid molecules of the present invention that do not encode a polypeptide having Nodal or Lefty activity include, inter alia, (1) isolating the Nodal or Lefty genes or allelic variants thereof in a cDNA library; (2) in situ hybridization (e.g., “FISH”) to metaphase chromosomal spreads to provide precise chromosomal location of the Nodal or Lefty genes, as described by Verma and colleagues (Human Chromosomes: A Manual of Basic Techniques, Pergamon Press, New York (1988)); and Northern Blot analysis for detecting Nodal or Lefty mRNA expression in specific tissues.

Preferred, however, are nucleic acid molecules having sequences at least 90%, 95%, 96%, 97%, 98% or 99% identical to the nucleic acid sequences shown in FIGS. 1A and B and 2A and B (SEQ ID NO:1 and SEQ ID NO:3, respectively) or to the nucleic acid sequences of the deposited cDNAs or to fragments of these polynucleotides as described herein, which do, in fact, encode polypeptides having Nodal or Lefty activity. By “a polypeptide having Nodal or Lefty activity” is intended polypeptides exhibiting activity similar, but not necessarily identical, to an activity of the active forms of Nodal or Lefty proteins of the invention, as measured in a particular biological assay. For example, the Nodal and Lefty proteins of the present invention are involved in the regulation of cell growth and differentiation. Other TGF-β-like molecules have the capacity to stimulate the proliferation of human endothelial cells in the presence of the comitogen concanavalin A (conA). Such an activity may be easily assayed by directly examining the effects of Nodal or Lefty or any muteins thereof on the proliferation of human endothelial cells as follows. Endothelial cells are obtained and cultured in 96 well flat-bottomed culture dishes (Costar, Cambridge, Mass.) in RPMI 1640 medium supplemented with 10% heat-inactivated fetal bovine serum (HyClone Labs, Logan, Utah), 1% L-glutamine, 100 U/mL penicillin, 100 μg/mL streptomycin, 0.1% gentamicin (Life Technologies, Inc., Rockville, Md.) in the presence of 2 μg/mL conA (Calbiochem, La Jolla, Calif.). ConA and the polypeptide to be analyzed are added to a final volume of medium of 0.2 mL. After 60 h at 37 C., cultures are pulsed with 1 μCi of [³H]-thymidine (5 Ci/mmol; 1 Ci=37 BGq; NEN) for 12-18 h and harvested onto glass fiber filters (PhD; Cambridge Technology, Watertown, Mass.). Mean [³H]-thymidine incorporation (CPM) of triplicate cultures is determined using a liquid scintillation counter (Beckman Instruments, Irvine, Calif.). Significant [³H]-thymidine incorporation indicates stimulation of endothelial cell proliferation. Such activity is useful for determining the potential for inducing or repressing the capacity for cellular growth and proliferation that Nodal or Lefty or a mutein thereof may possess.

Nodal and Lefty proteins regulate cellular proliferation and differentiation in a dose-dependent manner in the above-described assays. Although the compositions of the invention need not regulate cellular proliferation and differentiation in a dose-dependent manner, it is preferred that “a polypeptide having Nodal or Lefty activity” includes polypeptides that also exhibit any of the same cellular proliferation and differentiation regulatory activities in the above-described assays in a dose-dependent manner. Although the degree of dose-dependent activity need not be identical to that of the Nodal or Lefty proteins, preferably, “a polypeptide having Nodal or Lefty protein activity” will exhibit substantially similar dose-dependence in a given activity as compared to the Nodal or Lefty proteins (i.e., the candidate polypeptide will exhibit greater activity or not more than about 25-fold less and, preferably, not more than about tenfold less activity relative to the reference Nodal and Lefty proteins).

Further analysis of the ability of polypeptides of the invention to regulate cellular growth or differentiation of a particular cell type may be ascertained through the use of an in vitro colony forming assay to measure the extent of inhibition of myeloid progenitor cells (Youn, et al., J. Immunol. 155:2661-2667 (1995)). Briefly, this assay involves collecting human or mouse bone marrow cells and plating the same on agar, adding one or more growth factors and either (1) transfected host cell-supematant containing Nodal or Lefty protein (or a candidate polypeptide) or (2) nontransfected host cell-supematant control, and measuring the effect on colony formation by murine and human CFU-granulocyte-macrophages (CFU-GM), by human burst-forming unit-erythroid (BFU-E), or by human CFU granulocyte-erythroid-macrophage-megakaryocyte (CFU-GEMM).

Like other TGF-β-related molecules, Nodal and Lefty may exhibit an activity on leukocytes including, for example, monocytes, lymphocytes and neutrophils. For this reason, Nodal and Lefty are active in directing the proliferation and differentiation of these cell types. Such activity is useful, for example, for immune enhancement or suppression, myeloprotection, stem cell mobilization, acute and chronic inflammatory control and treatment of leukemia. Assays for measuring such activity are well known in the art (Peters, et al., Immun. Today 17:273 (1996); Young, et al., J. Exp. Med. 182:1111 (1995); Caux, et al., Nature 390:258 (1992); and Santiago-Schwarz, et al, Adv. Exp. Med. Biol. 378:7 (1995).

Of course, due to the degeneracy of the genetic code, one of ordinary skill in the art will immediately recognize that a large number of the nucleic acid molecules having a sequence at least 90%, 95%, 96%, 97%, 98%, or 99% identical to the nucleic acid sequence of the deposited cDNA or the nucleic acid sequences shown in FIGS. 1A and B and 2A and B (SEQ ID NO:1 and SEQ ID NO:3, respectively), or fragments thereof, will encode polypeptides “having Nodal or Lefty protein activity.” In fact, since degenerate variants of these nucleotide sequences all encode the same polypeptides, this will be clear to the skilled artisan even without performing the above described comparison assay. It will be further recognized in the art that, for such nucleic acid molecules that are not degenerate variants, a reasonable number will also encode a polypeptide having Nodal or Lefty activity. This is because the skilled artisan is fully aware of amino acid substitutions that are either less likely or not likely to significantly effect protein function (e.g., replacing one aliphatic amino acid with a second aliphatic amino acid), as further described below.

Polynucleotide Assays

The invention also encompasses the use of Nodal and Lefty polynucleotides to detect complementary polynucleotides, such as, for example, as a diagnostic reagent for detecting diseases or susceptibility to diseases related to the presence of mutated Nodal and Lefty. Such diseases are related to an under-expression of Nodal and Lefty, such as, for example, abnormal cellular proliferation such as tumors and cancers.

Individuals carrying mutations in the human Nodal or Lefty genes may be detected at the DNA level by a variety of techniques. Nucleic acids for diagnosis may be obtained from a patient's cells, such as from blood, urine, saliva, tissue biopsy and autopsy material. The genomic DNA may be used directly for detection or may be amplified enzymatically by using PCR (Saiki et al., Nature 324:163-166 (1986)) prior to analysis. RNA or cDNA may also be used for the same purpose. As an example, PCR primers complementary to the nucleic acid encoding Nodal or Lefty can be used to identify and analyze Nodal or Lefty mutations. For example, deletions and insertions can be detected by a change in size of the amplified product in comparison to the normal genotype. Point mutations can be identified by hybridizing amplified DNA to radiolabeled Nodal or Lefty RNA or alternatively, radiolabeled Nodal or Lefty antisense DNA sequences. Perfectly matched sequences can be distinguished from mismatched duplexes by RNase A digestion or by differences in melting temperatures.

Genetic testing based on DNA sequence differences may be achieved by detection of alteration in electrophoretic mobility of DNA fragments in gels with or without denaturing agents. Small sequence deletions and insertions can be visualized by high resolution gel electrophoresis. DNA fragments of different sequences may be distinguished on denaturing formamide gradient gels in which the mobilities of different DNA fragments are retarded in the gel at different positions according to their specific melting or partial melting temperatures (see, e.g., Myers et al., Science 230:1242 (1985)).

Sequence changes at specific locations may also be revealed by nuclease protection assays, such as RNase and S 1 protection or the chemical cleavage method (e.g., Cotton et al., Proc. Natl. Acad. Sci., USA, 85:4397-4401 (1985)).

Thus, the detection of a specific DNA sequence may be achieved by methods such as hybridization, RNase protection, chemical cleavage, direct DNA sequencing or the use of restriction enzymes, (e.g., Restriction Fragment Length Polymorphisms (RFLP)) and Southern blotting of genomic DNA.

In addition to more conventional gel-electrophoresis and DNA sequencing, mutations can also be detected by in situ analysis.

Vectors and Host Cells

While the Lefty and Nodal polypeptides (including fragments, variants derivatives, and analogs) of the invention can be chemically synthesized (e.g., see Creighton, 1983, Proteins: Structures and Molecular Principles, W. H. Freeman & Co., N.Y.), Lefty and Nodal polypeptides may advantageously be produced by recombinant DNA technology using techniques well known in the art for expressing gene sequences and/or nucleic acid coding sequences. Such methods can be used to construct expression vectors containing the polynucleotides of the invention and appropriate transcriptional and translational control signals. These methods include, for example, in vitro recombinant DNA techniques, synthetic techniques, and in vivo genetic recombination. See, for example, the techniques described in Sambrook et al., 1989, supra; Ausubel et al., 1989, supra; Caruthers et al., 1980, Nuc. Acids Res. Symp. Ser. 7:215-233; Crea and Horn, 1980, Nuc. Acids Res. 9(10):2331; Matteucci and Caruthers, 1980, Tetrahedron Letters 21:719; and Chow and Kempe, 1981, Nuc. Acids Res. 9(12):2807-2817. Alternatively, RNA capable of Lefty or Nodal sequences may be chemically synthesized using, for example, synthesizers. See, for example, the techniques described in “Oligonucleotide Synthesis”, 1984, Gait, M. J. ed., IRL Press, Oxford, which is incorporated by reference herein in its entirety.

Thus, in one embodiment, the present invention relates to vectors which include the isolated DNA molecules (i.e., polynucleotides) of the present invention, host cells which are genetically engineered with the recombinant vectors, and the production of Nodal or Lefty polypeptides or fragments thereof by recombinant techniques using these host cells or host cells that have otherwise been genetically engineered using techniques known in art to express a polypeptide of the invention. The vector may be, for example, a phage, plasmid, viral or retroviral vector. Retroviral vectors may be replication competent or replication defective. In the latter case, viral propagation generally will occur only in complementing host cells.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

In one embodiment, the polynucleotide of the invention is operatively associated with an appropriate heterologous regulatory element (e.g., a promoter or enhancer or both), such as the phage lambda PL promoter, the E. coli lac, trp, phoA and tac promoters, the SV40 early and late promoters and promoters of retroviral LTRs, to name a few. Other suitable promoters will be known to the skilled artisan.

In embodiments in which vectors contain expression constructs, these constructs will further contain sites for transcription initiation, termination and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS, 293 and Bowes melanoma cells; and plant cells. Appropriate culture mediums and conditions for the above-described host cells are known in the art.

Vectors preferred for use in bacteria include pHE4-5, pQE70, pQE60 and pQE-9 (QIAGEN, Inc., supra); pBS vectors, Phagescript vectors, Bluescript vectors, pNH8A, pNH16a, pNH18A, pNH46A (Stratagene); and ptrc99a, pKK223-3, pKK233-3, pDR540, pRIT5 (Pharmacia). Among preferred eukaryotic vectors are pWLNEO, pSV2CAT, pOG44, pXT1, and pSG (Stratagene); and pSVK3, pBPV, pMSG and pSVL (Pharmacia). Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals (for example, Davis, et al., Basic Methods In Molecular Biology (1986)).

In addition to encompassing host cells containing the vector constructs discussed herein, the invention also encompasses primary, secondary, and immortalized host cells of vertebrate origin, particularly those of mammalian origin, that have been engineered to delete or replace endogenous genetic material (e.g., Human Nodal or Human Lefty coding sequence), and/or to include genetic material (e.g. heterologous polynucleotide sequences) that is operably associated with Human Nodal or Human Lefty polynucleotides of the invention, and which activates, alters, and/or amplifies endogenous Human Nodal or Human Lefty polynucleotides. For example, techniques known in the art may be used to operably associate heterologous control regions (e.g. promoter and/or enhancer) and endogenous Human Nodal or Human Lefty polynucleotide sequences via homologous recombination (see, e.g. U.S. Pat. No. 5,641,670, issued Jun. 24, 1997; International Publication No. WO 96/29411, published Sep. 26, 1996; International Publication No. WO 94/12650, published Aug. 4, 1994; Koller et al., Proc. Natl. Acad. Sci. USA 86:8932-8935 (1989); and Zijlstra, et al., Nature 342:435-438 (1989), the disclosures of each of which are hereby incorporated by reference in their entireties).

The polypeptide may be expressed in a modified form, such as a fusion protein, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art. A preferred fusion protein comprises a heterologous region from immunoglobulin that is useful to stabilize and purify proteins. For example, EP-A-O 464 533 (Canadian counterpart 2045869) discloses fusion proteins comprising various portions of constant region of immunoglobulin molecules together with another human protein or part thereof. In many cases, the Fc part in a fusion protein is thoroughly advantageous for use in therapy and diagnosis and thus results, for example, in improved pharmacokinetic properties (EP-A 0232 262). On the other hand, for some uses it would be desirable to be able to delete the Fc part after the fusion protein has been expressed, detected and purified in the advantageous manner described. This is the case when Fc portion proves to be a hindrance to use in therapy and diagnosis, for example when the fusion protein is to be used as antigen for immunizations. In drug discovery, for example, human proteins, such as hIL-5, have been fused with Fc portions for the purpose of high-throughput screening assays to identify antagonists of hIL-5 (Bennett, D., et al., J. Molecular Recognition 8:52-58 (1995); Johanson, K., et al., J. Biol. Chem. 270:9459-9471 (1995)).

The Nodal and Lefty proteins can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification. Polypeptides of the present invention include: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins this prokaryotic removal process is inefficient, depending on the nature of the amino acid to which the N-terminal methionine is covalently linked.

Included within the scope of the invention are Lefty and Nodal polypeptides (including fragments, variants, derivatives and analogs) which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including, but not limited to, specific chemical cleavage by cyanogen bromide, trypsin, chymotrypsin, papain, V8 protease, NaBH4; acetylation, formylation, oxidation, reduction; metabolic synthesis in the presence of tunicamycin; etc. In a specific embodiment, the compositions of the invention are conjugated to other molecules to increase their water-solubility (e.g., polyethylene glycol), half-life, or ability to bind targeted tissue (e.g., bisphosphonates and fluorochromes to target the proteins to bony sites).

### POLYPEPTIDES AND FRAGMENTS

The invention further provides isolated Nodal and Lefty polypeptides having the amino acid sequences encoded by the deposited cDNAs, or the amino acid sequences in SEQ ID NO:2 and SEQ ID NO:4, respectively, or a peptide or polypeptide comprising a fragment (i.e., a portion) of the above polypeptides.

The polypeptides and polynucleotides of the present invention are preferably provided in an isolated form, and preferably are purified to a point within the range of near complete (e.g., >90% pure) to complete (e.g., >99% pure) homogeneity. The term “isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated. Also intended as an “isolated polypeptide” are polypeptides that have been purified partially or substantially from a recombinant host cell. For example, a recombinantly produced version of a Nodal or Lefty polypeptide can be substantially purified by the one-step method described by Smith and Johnson (Gene 67:31-40 (1988)). Such polynucleotides could be part of a vector and/or such polynucleotides or polypeptides could be part of a composition, and still be isolated in that such vector or composition is not part of its natural environment. Isolated polypeptides and polynucleotides according to the present invention also include such molecules produced naturally or synthetically. Polypeptides and polynucleotides of the invention also can be purified from natural or recombinant sources using anti-Nodal or anti-Lefty antibodies of the invention which may routinely be generated and utilized using methods known in the art.

To improve or alter the characteristics of Nodal and Lefty polypeptides, protein engineering may be employed. Recombinant DNA technology known to those skilled in the art can be used to create novel mutant proteins or muteins including single or multiple amino acid substitutions, deletions, additions or fusion proteins. Such modified polypeptides can show, e.g., enhanced activity or increased stability. In addition, they may be purified in higher yields and show better solubility than the corresponding natural polypeptide, at least under certain purification and storage conditions.

The present invention also encompasses fragments of the above-described Nodal and Lefty polypeptides. Polypeptide fragments of the present invention include polypeptides comprising an amino acid sequence contained in SEQ ID NO:2, SEQ ID NO:4, encoded by the cDNA contained in the deposited clones (HTLFA20 and HNGEF08, (encoding Nodal) and HUKEJ46 (encoding Lefty)), or encoded by nucleic acids which hybridize (e.g., under stringent hybridization conditions) to the nucleotide sequence contained in the deposited clones, that shown in FIGS. 1A and 1B (SEQ ID NO:1) and/or FIGS. 2A and 2B (SEQ ID NO:3), or the complementary strand thereto.

Polypeptide fragments may be “freestanding” or comprised within a larger polypeptide of which the fragment forms a part or region, most preferably as a single continuous region. Representative examples of polypeptide fragments of the invention, included, for example, fragments that comprise or alternatively, consist of, from about amino acid residues, 1 to 20, 21 to 40, 41 to 60, 61 to 83, 84 to 100, 101 to 120, 121 to 140, 141 to 160, 161 to 180, 181 to 200, 201 to 220, 201 to 224, 210 to 231, 221 to 240, 241 to 260, 261 to 280, 261 to 283, 281 to 289, 281 to 300, 301 to 320, 321 to 340, 341 to 348, 341 to 360, and 341 to 366 of SEQ ID NO:2 and/or SEQ ID NO:4. Moreover, polypeptide fragments can be at least about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350 or 360 amino acids in length. In this context “about” includes the particularly recited ranges, larger or smaller by several (i.e. 5, 4, 3, 2 or 1) amino acids, at either extreme or at both extremes.

In other embodiments, the fragments or polypeptides of the invention (i.e., those described herein) are not larger than 325, 300, 250, 225, 200, 185, 175, 170, 165, 160, 155, 150, 145, 140, 135, 130, 125, 120, 115, 110, 105, 100,90, 80, 75, 60, 50,40,30 or 25 amino acids residues in length.

Additional embodiments encompass polypeptide fragments comprising one or more functional regions of Nodal or Lefty polypeptides of the invention, such as, one or more Garnier-Robson alpha-regions, beta-regions, turn-regions, and coil-regions, Chou-Fasman alpha-regions, beta-regions, and coil-regions, Kyte-Doolittle hydrophilic regions and hydrophobic regions, Eisenberg alpha- and beta-amphipathic regions, Karplus-Schulz flexible regions, Emini surface-forming regions and Jameson-Wolf regions of high antigenic index, or any combination thereof, as disclosed in FIGS. 5 and 6 and in Tables I and II and as described herein.

Further preferred embodiments encompass polypeptide fragments comprising, or alternatively consisting of, the TGF-β-like domain of Nodal (amino acid residues 174-283 of SEQ ID NO:2).

Additional preferred embodiments encompass polypeptide fragments comprising, or alternatively consisting of, the mature domain of Lefty (amino acid residues 1-348 of SEQ ID NO:4), the first predicted TGF-β-like domain of Lefty (amino acid residues 60-348 of SEQ ID NO:4), the second predicted TGF-β-like domain of Lefty (amino acid residues 118-348 of SEQ ID NO:4), and/or the third predicted TGF-β-like domain of Lefty (amino acid residues 125-348 of SEQ ID NO:4).

In specific embodiments, polypeptide fragments of the invention comprise, or alternatively, consist of, amino acid residues aspartic acid-1 to alanine-27, arginine-30 to glutamic acid-58, cysteine-64 to phenylalanine-82, glycine-85 to serine-110, and leucine-130 to leucine-283 of the Nodal sequence recited in SEQ ID NO:2. In additional specific embodiments, polypeptide fragments of the invention comprise, or alternatively, consist of, amino acid residues leucine-(−15) to serine-(−2), alanine-3 to leucine-19, valine-34 to histidine-51, arginine-54 to leucine-72, glutamic acid-75 to arginine-114, arginine-117 to proline-192, histidine-198 to proline-209, glycine-211 to leucine-286, tryptophan-290 to glutamic acid-302, and serine-305 to proline-348 of the Lefty amino acid sequence recited in SEQ ID NO:4. These domains are regions of high identity identified by comparison of the TNF family member polypeptides shown in FIGS. 3 and 4.

In additional specific embodiments, the polypeptides of the invention comprise, or alternatively consist of, amino acid residues 19 to 25, 84 to 104, 105-125, 126 to 150, 151 to 170, 171 to 200, 201-250, 251 to 270, 271 to 297, 329 to 339, and/or 340 363 of the Lefty amino acid sequence depicted in FIGS. 2A and 2B. Polynucleotides encoding these polypeptides are also encompassed by the invention, as are polynucleotides that hybridize to the complementary strand of these encoding polynucleotides under high stringency conditions (e.g., as described herein) and polypeptides encoded by these hybridizing polynucleotides.

The polypeptides of the present invention have uses which include, but are not limited to, a molecular weight marker on SDS-PAGE gels or on molecular sieve gel filtration columns using methods well known to those of skill in the art.

As described in detail below, the polypeptides of the present invention can also be used to raise polyclonal and monoclonal antibodies, which are useful in assays for detecting Nodal or Lefty protein expression as described below or as agonists and antagonists capable of enhancing or inhibiting Nodal or Lefty protein function. Further, such polypeptides can be used in the yeast two-hybrid system to “capture” Nodal or Lefty protein binding proteins which are also candidate agonists and antagonists according to the present invention. The yeast two hybrid system is described by Fields and Song (Nature 340:245-246 (1989)).

In another embodiment, the invention provides peptides or polypeptides comprising epitope-bearing portions of a polypeptide of the invention. The epitope of this polypeptide portion is an immunogenic or antigenic epitope of a polypeptide of the invention. An “immunogenic epitope” is defined as a part of a protein that elicits an antibody response when the whole protein is the immunogen. On the other hand, a region of a protein molecule to which an antibody can bind is defined as an “antigenic epitope”. The number of immunogenic epitopes of a protein generally is less than the number of antigenic epitopes (see, for instance, Geysen, et al., Proc. Natl. Acad. Sci. USA 81:3998-4002 (1983)).

As to the selection of peptides or polypeptides bearing an antigenic epitope (i.e., that contain a region of a protein molecule to which an antibody can bind), it is well known in that art that relatively short synthetic peptides that mimic part of a protein sequence are routinely capable of eliciting an antiserum that reacts with the partially mimicked protein (see, for instance, Sutcliffe, J. G., et al., Science 219:660-666 (1983)). Peptides capable of eliciting protein-reactive sera are frequently represented in the primary sequence of a protein, can be characterized by a set of simple chemical rules, and are confined neither to immunodominant regions of intact proteins (i.e., immunogenic epitopes) nor to the amino or carboxyl terminals. Antigenic epitope-bearing peptides and polypeptides of the invention are therefore useful to raise antibodies, including monoclonal antibodies, that bind specifically to a polypeptide of the invention (see, for instance, Wilson, et al., Cell 37:767-778 (1984)).

Antigenic epitope-bearing peptides and polypeptides of the invention preferably contain a sequence of at least seven, more preferably at least nine and most preferably between about 15 to about 30 amino acids contained within the amino acid sequence of a polypeptide of the invention. Non-limiting examples of antigenic polypeptides or peptides that can be used to generate Nodal-specific antibodies include: a polypeptide comprising amino acid residues from about Lys-54 to about Asp-62, from about Val-91 to about Leu-99, from about Lys-100 to about Gln-108, from about Cys-116 to about Pro-124, from about Gln-140 to about Leu-148, from about Trp-156 to about Ser-164, from about Arg-170, to about Gln-181, from about Cys-212 to about Phe-224, from about Tyr-239, to about Thr-247, from about Pro-251, to about Met-259, and from about Asp-263, to about His-271. Non-limiting examples of antigenic polypeptides or peptides that can be used to generate Lefty-specific antibodies include: a polypeptide comprising amino acid residues from about Asp-71 to about Ser-79, from about Arg-106 to about Val-114, from about Leu-136 to about Arg-144, from about Asp-154 to about Asp-164, from about His-171 to about Asp-179, from about Gln-189 to about Leu-197, from about Pro-227 to about Glu-236, from about Gly-246 to about Glu-254, from about Pro-256 to about Gln-266, from about Cys-297 to about Ala-305, from about Ile-317 to about Pro-325, from about Ile-330 to about Val-340, and from about Val-348 to about Pro-366. These polypeptide fragments have been determined to bear antigenic epitopes of the Nodal and Lefty proteins by the analysis of the Jameson-Wolf antigenic index, as shown in FIGS. 5 and 6, and Tables I and II, above.

The epitope-bearing peptides and polypeptides of the invention may be produced by any conventional means (see, for example, Houghten, R. A., et al., Proc. Natl. Acad. Sci. USA 82:5131-5135 (1985); and U.S. Pat. No. 4,631,211 to Houghten, et al. (1986)).

Epitope-bearing peptides and polypeptides of the invention are used to induce antibodies according to methods well known in the art (see, for instance, Sutcliffe, et al., supra; Wilson, et al., supra; Chow, M., et al, Proc. Natl. Acad. Sci. USA 82:910-914; and Bittle, F. J., et al., J. Gen. Virol. 66:2347-2354 (1985)). Immunogenic epitope-bearing peptides of the invention, i.e., those parts of a protein that elicit an antibody response when the whole protein is the immunogen, are identified according to methods known in the art (see, for instance, Geysen, et al., supra). Further still, U.S. Pat. No. 5,194,392, issued to Geysen, describes a general method of detecting or determining the sequence of monomers (amino acids or other compounds) which is a topological equivalent of the epitope (i.e., a “mimotope”) which is complementary to a particular paratope (antigen binding site) of an antibody of interest. More generally, U.S. Pat. No. 4,433,092, issued to Geysen, describes a method of detecting or determining a sequence of monomers which is a topographical equivalent of a ligand which is complementary to the ligand binding site of a particular receptor of interest. Similarly, U.S. Pat. No. 5,480,971, issued to Houghten and colleagues, on Peralkylated Oligopeptide Mixtures discloses linear C1-C7-alkyl peralkylated oligopeptides and sets and libraries of such peptides, as well as methods for using such oligopeptide sets and libraries for determining the sequence of a peralkylated oligopeptide that preferentially binds to an acceptor molecule of interest. Thus, non-peptide analogs of the epitope-bearing peptides of the invention also can be made routinely by these methods.

For many proteins, including the extracellular domain of a membrane associated protein or the mature form(s) of a secreted protein, it is known in the art that one or more amino acids may be deleted from the N-terminus or C-terminus without substantial loss of biological function. For instance, Ron and colleagues (J.Biol. Chem., 268:2984-2988 (1993)) reported modified KGF proteins that had heparin binding activity even if 3, 8, or 27 N-terminal amino acid residues were missing. In the present case, since the Nodal and Lefty proteins of the invention are members of the TGF-β polypeptide superfamily, deletions of N-terminal amino acids up to the N-terminal-most cysteine of the predicted active form of the proteins at positions 183 and 233 of SEQ ID NO:2 and SEQ ID NO:4, respectively, may retain some biological activity such as receptor binding or modulation of target cell activities. Polypeptides having further N-terminal deletions including the Cys-183 and Cys-233 residues in SEQ ID NO:2 and SEQ ID NO:4, respectively, would not be expected to retain such biological activities because it is known that this residue in a TGF-β-related polypeptide is required for forming an integral part of the “cysteine knot motif” required for biological activities of the active form of TGF-β family members (McDonald, N. Q. and Hendrickson, W. A. Cell 73:303-304 (1993)).

However, even if deletion of one or more amino acids from the N-terminus of a protein results in modification of loss of one or more biological functions of the protein, other biological activities may still be retained. Thus, the ability of the shortened proteins to induce and/or bind to antibodies which recognize the complete or mature or active domains of the proteins generally will be retained when less than the majority of the residues of the complete or mature or active domains of the proteins are removed from the N-termini. Whether a particular polypeptide lacking N-terminal residues of a complete protein retains such immunologic activities can readily be determined by routine methods described herein and otherwise known in the art.

Accordingly, the present invention further provides polypeptides having one or more residues deleted from the amino terminus of the amino acid sequence of Nodal shown in SEQ ID NO:2, up to the cysteine residue at position number 183, and polynucleotides encoding such polypeptides. In particular, the present invention provides polypeptides comprising the amino acid sequence of residues n¹-283 of SEQ ID NO:2, where n¹ is an integer in the range of 173-183, and 183 is the position of the first residue from the N-terminus of the complete Nodal polypeptide (shown in SEQ ID NO:2) believed to be required for receptor binding activity of the Nodal protein.

More in particular, the invention provides polynucleotides encoding polypeptides having the amino acid sequence of residues of 173-283, 174-283, 175-283, 176-283, 177-283, 178-283, 179-283, 180-283, 181-283, 182-283, and 183-283 of SEQ ID NO:2. Polynucleotides encoding these polypeptides also are provided.

Further, the present invention also provides polypeptides having one or more residues deleted from the amino terminus of the amino acid sequence of Lefty shown in SEQ ID NO:4, up to the cysteine residue at position number 233, and polynucleotides encoding such polypeptides. In particular, the present invention provides polypeptides comprising the amino acid sequence of residues n²-348 of SEQ ID NO:4, where n² is an integer in the range of 125-233, and 233 is the position of the first residue from the N-terminus of the complete Nodal polypeptide (shown in SEQ ID NO:4) believed to be required for receptor binding activity of the Lefty protein.

More in particular, the invention provides polynucleotides encoding polypeptides having the amino acid sequence of residues of 125-348, 126-348, 127-348, 128-348, 129-348, 130-348, 131-348, 132-348, 133-348, 134-348, 135-348, 136-348, 137-348, 138-348, 139-348, 140-348, 141-348, 142-348, 143-348, 144-348, 145-348, 146-348, 147-348, 148-348, 149-348, 150-348, 151-348, 152-348, 153-348, 154-348, 155-348, 156-348, 157-348, 158-348, 159-348, 160-348, 161-348, 162-348, 163-348, 164-348, 165-348, 166-348, 167-348, 168-348, 169-348, 170-348, 171-348, 172-348, 173-348, 174-348, 175-348, 176-348, 177-348, 178-348, 179-348, 180-348, 181-348, 182-348, 183-348, 184-348, 185-348, 186-348, 187-348, 188-348, 189-348, 190-348, 191-348, 192-348, 193-348, 194-348, 195-348, 196-348, 197-348, 198-348, 199-348, 200-348, 201-348, 202-348, 203-348, 204-348, 205-348, 206-348, 207-348, 208-348, 209-348, 210-348, 211-348, 212-348, 213-348, 214-348, 215-348, 216-348, 217-348, 218-348, 219-348, 220-348, 221-348, 222-348, 223-348, 224-348, 225-348, 226-348, 227-348, 228-348, 229-348, 230-348, 231-348, 232-348, and 233-348 of SEQ ID NO:4. Polynucleotides encoding these polypeptides also are provided.

Similarly, many examples of biologically functional C-terminal deletion muteins are known. For instance, Interferon gamma shows up to ten times higher activities by deleting 8-10 amino acid residues from the carboxy terminus of the protein (Dobeli, et al., J. Biotechnology 7:199-216 (1988)). In the present case, since the proteins of the invention are members of the TGF-β polypeptide family, deletions of C-terminal amino acids up to the cysteine residues at positions 249 and 335 of SEQ ID NO:2 and SEQ ID NO:4, respectively, may retain some biological activity such as receptor binding or modulation of target cell activities. Polypeptides having further C-terminal deletions including Cys-249 and Cys-335 of SEQ ID NO:2 and SEQ ID NO:4, respectively, would not be expected to retain such biological activities because it is known that this residue in a TGF-β-related polypeptide is required for forming an integral part of the “cysteine knot motif” required for biological activities of the active form of TGF-β family members (McDonald, N. Q. and Hendrickson, W. A. Cell 73:303-304 (1993)).

However, even if deletion of one or more amino acids from the C-terminus of a protein results in modification of loss of one or more biological functions of the protein, other biological activities may still be retained. Thus, the ability of the shortened protein to induce and/or bind to antibodies which recognize the complete, mature or active forms of the protein generally will be retained when less than the majority of the residues of the complete, mature or active forms of the protein are removed from the C-terminus. Whether a particular polypeptide lacking C-terminal residues of a complete protein retains such immunologic activities can readily be determined by routine methods described herein and otherwise known in the art.

Accordingly, the present invention further provides polypeptides having one or more residues from the carboxy terminus of the amino acid sequence of Nodal shown in SEQ ID NO:2, up to the cysteine residue at position 249 of SEQ ID NO:2, and polynucleotides encoding such polypeptides. In particular, the present invention provides polypeptides having the amino acid sequence of residues 1-m¹ of the amino acid sequence in SEQ ID NO:2, where m¹ is any integer in the range of 249 to 283, and residue 249 is the position of the first residue from the C-terminus of the complete Nodal polypeptide (shown in SEQ ID NO:2) believed to be required for receptor binding or modulation of cellular growth and differentiation activities of the Nodal protein.

More in particular, the invention provides polynucleotides encoding polypeptides having the amino acid sequence of residues 1-249, 1-250, 1-251, 1-252, 1-253, 1-254, 1-255, 1-256, 1-257, 1-258, 1-259, 1-260, 1-261, 1-262, 1-263, 1-264, 1-265, 1-266, 1-267, 1-268, 1-269, 1-270, 1-271, 1-272, 1-273, 1-274, 1-275, 1-276, 1-277, 1-278, 1-279, 1-280, 1-281, 1-282, and 1-283 of SEQ ID NO:2. Polynucleotides encoding these polypeptides also are provided.

Further, the present invention also provides polypeptides having one or more residues from the carboxy terminus of the amino acid sequence of Lefty shown in SEQ ID NO:4, up to the cysteine residue at position 335 of SEQ ID NO:4, and polynucleotides encoding such polypeptides. In particular, the present invention provides polypeptides having the amino acid sequence of residues 1-m² of the amino acid sequence in SEQ ID NO:4, where m² is any integer in the range of 335 to 348, and residue 335 is the position of the first residue from the C-terminus of the complete Lefty polypeptide (shown in SEQ ID NO:4) believed to be required for receptor binding or modulation of cellular growth and differentiation activities of the Lefty protein.

More in particular, the invention provides polynucleotides encoding polypeptides having the amino acid sequence of residues 1-335, 1-336, 1-337, 1-338, 1-339, 1-340, 1-341, 1-342, 1-343, 1-344, 1-345, 1-346, 1-347, and 1-348 of SEQ ID NO:4. Polynucleotides encoding these polypeptides also are provided.

The invention also provides polypeptides having one or more amino acids deleted from both the amino and the carboxyl termini, which may be described generally as having residues n¹-m¹ of SEQ ID NO:2 or n²-m² SEQ ID NO:4, where n¹, m¹, n², and m² are integers as described above.

Also included is a nucleotide sequence encoding a polypeptide consisting of a portion of the complete Nodal amino acid sequence encoded by the cDNA clone contained in ATCC Deposit No. 209092 and/or 209135, where this portion excludes from 1 to about 183 amino acids from the amino terminus of the complete amino acid sequence encoded by the cDNA clone contained in ATCC Deposit No. 209092 and/or 209135, or from 1 to about 34 amino acids from the carboxy terminus, or any combination of the above amino terminal and carboxy terminal deletions, of the complete amino acid sequence encoded by the cDNA clone contained in ATCC Deposit No. 209092 and/or 209135.

In addition, a nucleotide sequence encoding a polypeptide consisting of a portion of the complete Lefty amino acid sequence encoded by the cDNA clone contained in ATCC Deposit No. 209091 is included, where this portion excludes from 1 to about 250 amino acids from the amino terminus of the complete amino acid sequence encoded by the cDNA clone contained in ATCC Deposit No. 209091, or from 1 to about 12 amino acids from the carboxy terminus, or any combination of the above amino terminal and carboxy terminal deletions, of the complete amino acid sequence encoded by the cDNA clone contained in ATCC Deposit No. 209091. Polynucleotides encoding all of the above deletion mutant polypeptide forms also are provided.

As mentioned above, even if deletion of one or more amino acids from the N-terminus of a protein results in modification of loss of one or more biological functions of the protein, other biological activities may still be retained. Thus, the ability of the shortened Human Nodal or Human Lefty mutein to induce and/or bind to antibodies which recognize the complete or mature of the protein generally will be retained when less than the majority of the residues of the complete or mature protein are removed from the N-terminus. Whether a particular polypeptide lacking N-terminal residues of a complete protein retains such immunologic activities can readily be determined by routine methods described herein and otherwise known in the art. It is not unlikely that a Human Nodal or Human Lefty mutein with a large number of deleted N-terminal amino acid residues may retain some biological or immungenic activities. In fact, peptides composed of as few as six Human Nodal or Human Lefty amino acid residues may often evoke an immune response.

Accordingly, the present invention further provides polypeptides having one or more residues deleted from the amino terminus of the Human Nodal amino acid sequence shown in SEQ ID NO:2, up to the glutamic acid residue at position number 278 and polynucleotides encoding such polypeptides. In particular, the present invention provides polypeptides comprising the amino acid sequence of residues n³-283 of FIGS. 1A and B (SEQ ID NO:2), where n³ is an integer in the range of 2 to 278, and 279 is the position of the first residue from the N-terminus of the complete Human Nodal polypeptide believed to be required for at least immunogenic activity of the Human Nodal protein.

More in particular, the invention provides polynucleotides encoding polypeptides comprising, or alternatively consisting of, the amino acid sequence of residues of V-2 to L-283; A-3 to L-283; V-4 to L-283; D-5 to L-283; G-6 to L-283; Q-7 to L-283; N-8 to L-283; W-9 to L-283; T-10 to L-283; F-11 to L-283; A-12 to L-283; F-13 to L-283; D-14 to L-283; F-15 to L-283; S-16 to L-283; F-17 to L-283; L-18 to L-283; S-19 to L-283; Q-20 to L-283; Q-21 to L-283; E-22 to L-283; D-23 to L-283; L-24 to L-283; A-25 to L-283; W-26 to L-283; A-27 to L-283; E-28 to L-283; L-29 to L-283; R-30 to L-283; L-31 to L-283; Q-32 to L-283; L-33 to L-283; S-34 to L-283; S-35 to L-283; P-36 to L-283; V-37 to L-283; D-38 to L-283; L-39 to L-283; P-40 to L-283; T-41 to L-283; E-42 to L-283; G-43 to L-283; S-44 to L-283; L-45 to L-283; A-46 to L-283; I-47 to L-283; E-48 to L-283; 1-49 to L-283; F-50 to L-283; H-51 to L-283; Q-52 to L-283; P-53 to L-283; K-54 to L-283; P-55 to L-283; D-56 to L-283; T-57 to L-283; E-58 to L-283; Q-59 to L-283; A-60 to L-283; S-61 to L-283; D-62 to L-283; S-63 to L-283; C-64 to L-283; L-65 to L-283; E-66 to L-283; R-67 to L-283; F-68 to L-283; Q-69 to L-283; M-70 to L-283; D-71 to L-283; L-72 to L-283; F-73 to L-283; T-74 to L-283; V-75 to L-283; T-76 to L-283; L-77 to L-283; S-78 to L-283; Q-79 to L-283; V-80 to L-283; T-81 to L-283; F-82 to L-283; S-83 to L-283; L-84 to L-283; G-85 to L-283; S-86 to L-283; M-87 to L-283; V-88 to L-283; L-89 to L-283; E-90 to L-283; V-91 to L-283; T-92 to L-283; R-93 to L-283; P-94 to L-283; L-95 to L-283; S-96 to L-283; K-97 to L-283; W-98 to L-283; L-99 to L-283; K-100 to L-283; R-101 to L-283; P-102 to L-283; G-103 to L-283; A-104 to L-283; L-105 to L-283; E-106 to L-283; K-107 to L-283; Q-108 to L-283; M-109 to L-283; S-110 to L-283; R-111 to L-283; V-112 to L-283; A-113 to L-283; G-114 to L-283; E-115 to L-283; C-116 to L-283; W-117 to L-283; P-118 to L-283; R-119 to L-283; P-120 to L-283; P-121 to L-283; T-122 to L-283; P-123 to L-283; P-124 to L-283; A-125 to L-283; T-126 to L-283; N-127 to L-283; V-128 to L-283; L-129 to L-283; L-130 to L-283; M-131 to L-283; L-132 to L-283; Y-133 to L-283; S-134 to L-283; N-135 to L-283; L-136 to L-283; S-137 to L-283; Q-138 to L-283; E-139 to L-283; Q-140 to L-283; R-141 to L-283; Q-142 to L-283; L-143 to L-283; G-144 to L-283; G-145 to L-283; S-146 to L-283; T-147 to L-283; L-148 to L-283; L-149 to L-283; W-150 to L-283; E-151 to L-283; A-152 to L-283; E-153 to L-283; S-154 to L-283; S-155 to L-283; W-156 to L-283; R-157 to L-283; A-158 to L-283; Q-159 to L-283; E-160 to L-283; G-161 to L-283; Q-162 to L-283; L-163 to L-283; S-164 to L-283; W-165 to L-283; E-166 to L-283; W-167 to L-283; G-168 to L-283; K-169 to L-283; R-170 to L-283; H-171 to L-283; R-172 to L-283; R-173 to L-283; H-174 to L-283; H-175 to L-283; L-176 to L-283; P-177 to L-283; D-178 to L-283; R-179 to L-283; S-180 to L-283; Q-181 to L-283; L-182 to L-283; C-183 to L-283; R-184 to L-283; K-185 to L-283; V-186 to L-283; K-187 to L-283; F-188 to L-283; Q-189 to L-283; V-190 to L-283; D-191 to L-283; F-192 to L-283; N-193 to L-283; L-194 to L-283; I-195 to L-283; G-196 to L-283; W-197 to L-283; G-198 to L-283; S-199 to L-283; W-200 to L-283; 1-201 to L-283; I-202 to L-283; Y-203 to L-283; P-204 to L-283; K-205 to L-283; Q-206 to L-283; Y-207 to L-283; N-208 to L-283; A-209 to L-283; Y-210 to L-283; R-211 to L-283; C-212 to L-283; E-213 to L-283; G-214 to L-283; E-215 to L-283; C-216 to L-283; P-217 to L-283; N-218 to L-283; P-219 to L-283; V-220 to L-283; G-221 to L-283; E-222 to L-283; E-223 to L-283; F-224 to L-283; H-225 to L-283; P-226 to L-283; T-227 to L-283; N-228 to L-283; H-229 to L-283; A-230 to L-283; Y-231 to L-283; 1-232 to L-283; Q-233 to L-283; S-234 to L-283; L-235 to L-283; L-236 to L-283; K-237 to L-283; R-238 to L-283; Y-239 to L-283; Q-240 to L-283; P-241 to L-283; H-242 to L-283; R-243 to L-283; V-244 to L-283; P-245 to L-283; S-246 to L-283; T-247 to L-283; C-248 to L-283; C-249 to L-283; A-250 to L-283; P-251 to L-283; V-252 to L-283; K-253 to L-283; T-254 to L-283; K-255 to L-283; P-256 to L-283; L-257 to L-283; S-258 to L-283; M-259 to L-283; L-260 to L-283; Y-261 to L-283; V-262 to L-283; D-263 to L-283; N-264 to L-283; G-265 to L-283; R-266 to L-283; V-267 to L-283; L-268 to L-283; L-269 to L-283; D-270 to L-283; H-271 to L-283; H-272 to L-283; K-273 to L-283; D-274 to L-283; M-275 to L-283; 1-276 to L-283; V-277 to L-283; and E-278 to L-283 of the Human Nodal sequence shown in FIGS. 1A and B (which is identical to the Human Nodal sequence in SEQ ID NO:2). Polynucleotides encoding these polypeptides are also encompassed by the invention.

Also as mentioned above, even if deletion of one or more amino acids from the C-terminus of a protein results in modification of loss of one or more biological functions of the protein, other biological activities may still be retained. Thus, the ability of the shortened Human Nodal mutein to induce and/or bind to antibodies which recognize the complete or mature of the protein generally will be retained when less than the majority of the residues of the complete or mature protein are removed from the C-terminus. Whether a particular polypeptide lacking C-terminal residues of a complete protein retains such immunologic activities can readily be determined by routine methods described herein and otherwise known in the art. It is not unlikely that a Human Nodal imutein with a large number of deleted C-terminal amino acid residues may retain some biological or immungenic activities. In fact, peptides composed of as few as six Human Nodal amino acid residues may often evoke an immune response.

Accordingly, the present invention further provides polypeptides having one or more residues deleted from the carboxy terminus of the amino acid sequence of the Human Nodal shown in SEQ ID NO:2, up to the glycine residue at position number 6, and polynucleotides encoding such polypeptides. In particular, the present invention provides polypeptides comprising the amino acid sequence of residues 1-m³ of SEQ ID NO:2, where m³ is an integer in the range of 6 to 283, and 6 is the position of the first residue from the C-terminus of the complete Human Nodal polypeptide believed to be required for at least immunogenic activity of the Human Nodal protein.

More in particular, the invention provides polynucleotides encoding polypeptides comprising, or alternatively consisting of, the amino acid sequence of residues D-1 to C-282; D-1 to G-281; D-1 to C-280; D-1 to E-279; D-1 to E-278; D-1 to V-277; D-1 to 1-276; D-1 to M-275; D-1 to D-274; D-1 to K-273; D-1 to H-272; D-1 to H-271; D-1 to D-270; D-1 to L-269; D-1 to L-268; D-1 to V-267; D-1 to R-266; D-1 to G-265; D-1 to N-264; D-1 to D-263; D-1 to V-262; D-1 to Y-261; D-1 to L-260; D-1 to M-259; D-1 to S-258; D-1 to L-257; D-1 to P-256; D-1 to K-255; D-1 to T-254; D-1 to K-283; D-1 to V-252; D-1 to P-251; D-1 to A-250; D-1 to C-249; D-1 to C-248; D-1 to T-247; D-1 to S-246; D-1 to P-245; D-1 to V-244; D-1 to R-243; D-1 to H-242; D-1 to P-241; D-1 to Q-240; D-1 to Y-239; D-1 to R-238; D-1 to K-237; D-1 to L-236; D-1 to L-235; D-1 to S-234; D-1 to Q-233; D-1 to 1-232; D-1 to Y-231; D-1 to A-230; D-1 to H-229; D-1 to N-228; D-1 to T-227; D-1 to P-226; D-1 to H-225; D-1 to F-224; D-1 to E-223; D-1 to E-222; D-1 to G-221; D-1 to V-220; D-1 to P-219; D-1 to N-218; D-1 to P-217; D-1 to C-216; D-1 to E-215; D-1 to G-214; D-1 to E-213; D-1 to C-212; D-1 to R-211; D-1 to Y-210; D-1 to A-209; D-1 to N-208; D-1 to Y-207; D-1 to Q-206; D-1 to K-205; D-1 to P-204; D-1 to Y-203; D-1 to 1-202; D-1 to 1-201; D-1 to W-200; D-1 to S-199; D-1 to G-198; D-1 to W-197; D-1 to G-196; D-1 to 1-195; D-1 to L-194; D-1 to N-193; D-1 to F-192; D-1 to D-191; D-1 to V-190; D-1 to Q-189; D-1 to F-188; D-1 to K-187; D-1 to V-186; D-1 to K-185; D-1 to R-184; D-1 to C-183; D-1 to L-182; D-1 to Q-181; D-1 to S-180; D-1 to R-179; D-1 to D-178; D-1 to P-177; D-1 to L-176; D-1 to H-175; D-1 to S-174; D-1 to R-173; D-1 to R-172; D-1 to P-171; D-1 to R-170; D-1 to K-169; D-1 to G-168; D-1 to W-167; D-1 to E-166; D-1 to W-165; D-1 to S-164; D-1 to L-163; D-1 to Q-162; D-1 to G-161; D-1 to E-160; D-1 to Q-159; D-1 to A-158; D-1 to R-157; D-1 to W-156; D-1 to S-155; D-1 to S-154; D-1 to E-153; D-1 to A-152; D-1 to E-151; D-1 to W-150; D-1 to L-149; D-1 to L-148; D-1 to T-147; D-1 to S-146; D-1 to G-145; D-1 to G-144; D-1 to L-143; D-1 to Q-142; D-1 to R-141; D-1 to Q-140; D-1 to E-139; D-1 to Q-138; D-1 to S-137; D-1 to L-136; D-1 to N-135; D-1 to S-134; D-1 to Y-133; D-1 to L-132; D-1 to M-131; D-1 to L-130; D-1 to L-129; D-1 to V-128; D-1 to N-127; D-1 to T-126; D-1 to A-125; D-1 to P-124; D-1 to P-123; D-1 to T-122; D-1 to P-121; D-1 to P-120; D-1 to R-119; D-1 to P-118; D-1 to W-117; D-1 to C-116; D-1 to E-115; D-1 to G-114; D-1 to A-113; D-1 to V-112; D-1 to R-111; D-1 to S-140; D-1 to M-109; D-1 to Q-108; D-1 to K-107; D-1 to E-106; D-1 to L-105; D-1 to A-104; D-1 to G-103; D-1 to P-102; D-1 to R-101; D-1 to K-100; D-1 to L-99; D-1 to W-98; D-1 to K-97; D-1 to S-96; D-1 to L-95; D-1 to P-94; D-1 to R-93; D-1 to T-92; D-1 to V-91; D-1 to to E-90; D-1 to L-89; D-1 to V-88; D-1 to M-87; D-1 to S-86; D-1 to G-85; D-85; D-1 to L-84; D-1 to S-83; D-1 to F-82; D-1 to T-81; D-1 to V-80; D-1 to Q-79; D-1 to S-78; D-1 to L-77; D-1 to T-76; D-1 to V-75; D-1 to T-74; D-1 to F-73; D-1 to L-72; D-1 to D-71; D-1 to M-70; D-1 to Q-69; D-1 to F-68; D-1 to R-67; D-1 to E-66; D-1 to L-65; D-1 to C-64; D-1 to S-63; D-1 to D-62; D-1 to S-61; D-1 to A-60; D-1 to Q-59; D-1 to E-58; D-1 to T-57; D-1 to D-56; D-1 to P-55; D-1 to K-54; D-1 to P-53; D-1 to Q-52; D-1 to H-51; D-1 to F-50; D-1 to I-49; D-1 to E-48; D-1 to 1-47; D-1 to A-46; D-1 to L-45; D-1 to S-44; D-1 to G-43; D-1 to E-42; D-1 to T-41; D-1 to P-40; D-1 to L-39; D-1 to D-38; D-1 to V-37; D-1 to P-36; D-1 to S-35; D-1 to S-34; D-1 to L-33; D-1 to Q-32; D-1 to L-31; D-1 to R-30; D-1 to L-29; D-1 to E-28; D-1 to A-27; D-1 to W-26; D-1 to A-25; D-1 to L-24; D-1 to D-23; D-1 to E-22; D-1 to Q-21; D-1 to Q-20; D-1 to S-19; D-1 to L-18; D-1 to F-17; D-1 to S-16; D-1 to F-15; D-1 to D-14; D-1 to F-13; D-1 to A-12; D-1 to F-11; D-1 to T-10; D-1 to W-9; D-1 to N-8; D-1 to Q-7; D-1 to G-6 of the sequence of the Human Nodal sequence shown in FIGS. 1A and B (which is identical to the Human Nodal sequence shown in SEQ ID NO:2). Polynucleotides encoding these polypeptides also are provided.

The invention also provides polypeptides having one or more amino acids deleted from both the amino and the carboxyl termini of a Human Nodal polypeptide, which may be described generally as having residues n³-m³ of FIGS. 1A and B (SEQ ID NO:2), where n³ and m³ are integers as described above.

Again as mentioned above, even if deletion of one or more amino acids from the N-terminus of a protein results in modification of loss of one or more biological functions of the protein, other biological activities may still be retained. Thus, the ability of the shortened Human Lefty mutein to induce and/or bind to antibodies which recognize the complete or mature of the protein generally will be retained when less than the majority of the residues of the complete or mature protein are removed from the N-terminus. Whether a particular polypeptide lacking N-terminal residues of a complete protein retains such immunologic activities can readily be determined by routine methods described herein and otherwise known in the art. It is not unlikely that a Human Lefty mutein with a large number of deleted N-terminal amino acid residues may retain some biological or immungenic activities. In fact, peptides composed of as few as six Human Lefty amino acid residues may often evoke an immune response.

Accordingly, the present invention further provides polypeptides having one or more residues deleted from the amino terminus of the Human Lefty amino acid sequence shown in SEQ ID NO:4, up to the proline residue at position number 361 and polynucleotides encoding such polypeptides. In particular, the present invention provides polypeptides comprising the amino acid sequence of residues n⁴-180 of FIGS. 2A and B (SEQ ID NO:4), where n⁴ is an integer in the range of 2 to 361, and 362 is the position of the first residue from the N-terminus of the complete Human Lefty polypeptide believed to be required for at least immunogenic activity of the Human Lefty protein.

More in particular, the invention provides polynucleotides encoding polypeptides comprising, or alternatively consisting of, the amino acid sequence of residues of Q-2 to P-366; P-3 to P-366; L-4 to P-366; W-5 to P-366; L-6 to P-366; C-7 to P-366; W-8 to P-366; A-9 to P-366; L-10 to P-366; W-11 to P-366; V-12 to P-366; L-13 to P-366; P-14 to P-366; L-15 to P-366; A-16 to P-366; S-17 to P-366; P-18 to P-366; G-19 to P-366; A-20 to P-366; A-21 to P-366; L-22 to P-366; T-23 to P-366; G-24 to P-366; E-25 to P-366; Q-26 to P-366; L-27 to P-366; L-28 to P-366; G-29 to P-366; S-30 to P-366; L-31 to P-366; L-32 to P-366; R-33 to P-366; Q-34 to P-366; L-35 to P-366; Q-36 to P-366; L-3 7 to P-366 ; K-3 8 to P-366 ; E-3 9 to P-366 ; V-40 to P-366 ; P-41 to P-366; T-42 to P-366; L-43 to P-366; D-44 to P-366; R-45 to P-366; A-46 to P-366; D-47 to P-366; M-48 to P-366; E-49 to P-366; E-50 to P-366; L-51 to P-366; V-52 to P-366; I-53 to P-366; P-54 to P-366; T-55 to P-366; H-56 to P-366; V-57 to P-366; R-58 to P-366; A-59 to P-366; Q-60 to P-366; Y-61 to P-366; V-62 to P-366; A-63 to P-366; L-64 to P-366; L-65 to P-366; Q-66 to P-366; R-67 to P-366; S-68 to P-366; H-69 to P-366; G-70 to P-366; D-71 to P-366; R-72 to P-366; S-73 to P-366; R-74 to P-366; G-75 to P-366; K-76 to P-366; R-77 to P-366; F-78 to P-366; S-79 to P-366; Q-80 to P-366; S-81 to P-366; F-82 to P-366; R-83 to P-366; E-84 to P-366; V-85 to P-366; A-86 to P-366; G-87 to P-366; R-88 to P-366; F-89 to P-366; L-90 to P-366; A-91 to P-366; L-92 to P-366; E-93 to P-366; A-94 to P-366; S-95 to P-366; T-96 to P-366; H-97 to P-366; L-98 to P-366; L-99 to P-366; V-100 to P-366; F-101 to P-366; G-102 to P-366; M-103 to P-366; E-104 to P-366; Q-105 to P-366; R-106 to P-366; L-107 to P-366; P-i08 to P-366; P-109 to P-366; N-i 10 to P-366; S-i 1 1 to P-366; E-1 12 to P-366; L-1 13 to P-366; V-114 to P-366; Q-115 to P-366; A-116 to P-366; V-117 to P-366; L-118 to P-366; R-119 to P-366; L-120 to P-366; F-121 to P-366; Q-122 to P-366; E-123 to P-366; P-124 to P-366; V-325 to P-366; P-126 to P-366; K-127 to P-366; A-128 to P-366; A-129 to P-366; L-130 to P-366; H-131 to P-366; R-132 to P-366; H-133 to P-366; G-134 to P-366; R-135 to P-366; L-136 to P-366; S-137 to P-366; P-138 to P-366; R-139 to P-366; S-140 to P-366; A-124 to P-366; R-142 to P-366; A-143 to P-366; R-144 to P-366; V-145 to P-366; T-146 to P-366; V-147 to P-366; E-148 to P-366; W-149 to P-366; L-150 to P-366; R-151 to P-366; V-152 to P-366; R-153 to P-366; D-154 to P-366; D-155 to P-366; G-156 to P-366; S-157 to P-366; N-158 to P-366; R-159 to P-366; T-160 to P-366; S-161 to P-366; L-162 to P-366; 1-163 to P-366; D-164 to P-366; S-165 to P-366; R-166 to P-366; L-167 to P-366; V-168 to P-366; S-169 to P-366; V-170 to P-366; H-171 to P-366; E-172 to P-366; S-173 to P-366; G-174 to P-366; W-175 to P-366; K-176 to P-366; A-177 to P-366; F-178 to P-366; D-179 to P-366; V-180 to P-366; T-181 to P-366; E-182 to P-366; A-183 to P-366; V-184 to P-366; N-185 to P-366; F-186 to P-366; W-187 to P-366; Q-188 to P-366; Q-189 to P-366; L-190 to P-366; S-191 to P-366; R-192 to P-366; P-193 to P-366; R-194 to P-366; Q-195 to P-366; P-196 to P-366; L-197 to P-366; L-198 to P-366; L-199 to P-366; Q-200 to P-366; V-201 to P-366; S-202 to P-366; V-203 to P-366; Q-204 to P-366; R-205 to P-366; E-206 to P-366; H-207 to P-366; L-208 to P-366; G-209 to P-366; P-2F0 to P-366; L-211 to P-366; A-212 to P-366; S-213 to P-366; G-214 to P-366; A-215 to P-366; H-216 to P-366; K-217 to P-366; L-218 to P-366; V-219 to P-366; R-220 to P-366; F-221 to P-366; A-222 to P-366; S-223 to P-366; Q-224 to P-366; G-225 to P-366; A-226 to P-366; P-227 to P-366; A-228 to P-366; G-229 to P-366; L-230 to P-366; G-231 to P-366; E-232 to P-366; P-233 to P-366; Q-234 to P-366; L-235 to P-366; E-236 to P-366; L-237 to P-366; 11-238 to P-366; T-239 to P-366; L-240 to P-366; D-241 to P-366; L-242 to P-366; G-243 to P-366; D-244 to P-366; Y-245 to P-366; G-246 to P-366; A-247 to P-366; Q-248 to P-366; G-249 to P-366; D-250 to P-366; C-251 to P-366; D-252 to P-366; P-253 to P-366; E-254 to P-366; A-255 to P-366; P-256 to P-366; M-257 to P-366; T-258 to P-366; E-259 to P-366; G-260 to P-366; T-261 to P-366; R-262 to P-366; C-263 to P-366; C-264 to P-366; R-265 to P-366; Q-266 to P-366; E-267 to P-366; M-268 to P-366; Y-269 to P-366; 1-270 to P-366; D-271 to P-366; L-272 to P-366; Q-273 to P-366; G-274 to P-366; M-275 to P-366; K-276 to P-366; W-277 to P-366; A-278 to P-366; E-279 to P-366; N-280 to P-366; W-281 to P-366; V-282 to P-366; L-283 to P-366; E-284 to P-366; P-285 to P-366; P-286 to P-366; G-287 to P-366; F-288 to P-366; L-289 to P-366; A-290 to P-366; Y-291 to P-366; E-292 to P-366; C-293 to P-366; V-294 to P-366; G-295 to P-366; T-296 to P-366; C-297 to P-366; R-298 to P-366; Q-299 to P-366; P-300 to P-366; P-301 to P-366; E-302 to P-366; A-303 to P-366; L-304 to P-366; A-305 to P-366; F-306 to P-366; K-307 to P-366; W-308 to P-366; P-309 to P-366; F-310 to P-366; L-321 to P-366; G-312 to P-366; P-313 to P-366; R-314 to P-366; Q-315 to P-366; C-316 to P-366; I-317 to P-366; A-318 to P-366; S-319 to P-366; E-320 to P-366; T-321 to P-366; D-322 to P-366; S-323 to P-366; L-324 to P-366; P-325 to P-366; M-326 to P-366; 1-327 to P-366; V-328 to P-366; S-329 to P-366; 1-330 to P-366; K-331 to P-366; E-332 to P-366; G-333 to P-366; G-334 to P-366; R-335 to P-366; T-336 to P-366; R-337 to P-366; P-338 to P-366; Q-339 to P-366; V-340 to P-366; V-341 to P-366; S-342 to P-366; L-343 to P-366; P-344 to P-366; N-345 to P-366; M-346 to P-366; R-347 to P-366; V-348 to P-366; Q-349 to P-366; K-350 to P-366; C-351 to P-366; S-352 to P-366; C-353 to P-366; A-354 to P-366; S-355 to P-366; D-356 to P-366; G-357 to P-366; A-358 to P-366; L-359 to P-366; V-360 to P-366; and P-361 to P-366 of the Human Lefty sequence shown in FIGS. 2A and B (which is identical to the sequence shown as SEQ ID NO:4, with the exception that the amino acid residues in FIGS. 2A and B are numbered consecutively from 1 through 366 from the N-terminus to the C-terminus, while the amino acid residues in SEQ ID NO:4 are numbered consecutively from −18 through 348 to reflect the position of the predicted signal peptide). Polynucleotides encoding these polypeptides are also encompassed by the invention.

Also as mentioned above, even if deletion of one or more amino acids from the C-terminus of a protein results in modification of loss of one or more biological functions of the protein, other biological activities may still be retained. Thus, the ability of the shortened Human Lefty mutein to induce and/or bind to antibodies which recognize the complete or mature of the protein generally will be retained when less than the majority of the residues of the complete or mature protein are removed from the C-terminus. Whether a particular polypeptide lacking C-terminal residues of a complete protein retains such immunologic activities can readily be determined by routine methods described herein and otherwise known in the art. It is not unlikely that a Human Lefty mutein with a large number of deleted C-terminal amino acid residues may retain some biological or immungenic activities. In fact, peptides composed of as few as six Human Lefty amino acid residues may often evoke an immune response.

Accordingly, the present invention further provides polypeptides having one or more residues deleted from the carboxy terminus of the amino acid sequence of the Human Lefty shown in SEQ ID NO:4, up to the leucine residue at position number 6, and polynucleotides encoding such polypeptides. In particular, the present invention provides polypeptides comprising the amino acid sequence of residues 1-m⁴ of SEQ ID NO:4, where m⁴ is an integer in the range of 6 to 366, and 6 is the position of the first residue from the C-terminus of the complete Human Lefty polypeptide believed to be required for at least immunogenic activity of the Human Lefty protein.

More in particular, the invention provides polynucleotides encoding polypeptides comprising, or alternatively consisting of, the amino acid sequence of residues M-1 to Q-365; M-1 to L-364; M-1 to R-363; M-1 to R-362; M-1 to P-361; M-1 to V-360; M-1 to L-359; M-1 to A-358; M-1 to G-357; M-1 to D-356; M-1 to S-355; M-1 to A-354; M-1 to C-353; M-1 to S-352; M-1 to C-351; M-1 to K-350; M-1 to Q-349; M-1 to V-348; M-1 to R-347; M-1 to M-346; M-1 to N-345; M-1 to P-344; M-1 to L-343; M-1 to S-342; M-1 to V-341; M-1 to V-340; M-1 to Q-339; M-1 to P-338; M-1 to R-337; M-1 to T-336; M-1 to R-335; M-1 to G-334; M-1 to G-333; M-1 to E-332; M-1 to K-331; M-1 to I-330; M-1 to S-329; M-1 to V-328; M-1 to I-327; M-1 to M-326; M-1 to P-325; M-1 to L-324; M-1 to S-323; M-1 to D-322; M-1 to T-321; M-1 to E-320; M-1 to S-319; M-1 to A-318; M-1 to I-317; M-1 to C-316; M-1 to Q-315; M-1 to R-314; M-1 to P-313; M-1 to G-312; M-1 to L-311; M-1 to F-310; M-1 to P-309; M-1 to W-308; M-1 to K-307; M-1 to F-306; M-1 to A-305; M-1 to L-304; M-1 to A-303; M-1 to E-302; M-1 to P-301; M-1 to P-300; M-1 to Q-299; M-1 to R-298; M-1 to C-297; M-1 to T-296; M-1 to G-295; M-1 to V-294; M-1 to C-293; M-1 to E-292; M-1 to Y-291; M-1 to A-290; M-1 to L-289; M-1 to F-288; M-1 to G-287; M-1 to P-286; M-1 to P-285; M-1 to E-284; M-1 to L-283; M-1 to V-282; M-1 to W-281; M-1 to N-280; M-1 to E-279; M-1 to A-278; M-1 to W-277; M-1 to K-276; M-1 to M-275; M-1 to G-274; M-1 to Q-273; M-1 to L-272; M-1 to D-271; M-1 to I-270; M-1 to Y-269; M-1 to M-268; M-1 to E-267; M-1 to Q-266; M-1 to R-265; M-1 to C-264; M-1 to C-263; M-1 to R-262; M-1 to T-261; M-1 to G-260; M-1 to E-259; M-1 to T-258; M-1 to M-257; M-1 to P-256; M-1 to A-255; M-1 to E-254; M-1 to P-253; M-1 to D-252; M-1 to C-251; M-1 to D-250; M-1 to G-249; M-1 to Q-248; M-1 to A-247; M-1 to G-246; M-1 to Y-245; M-1 to D-244; M-1 to G-243; M-1 to L-242; M-1 to D-241; M-1 to L-240; M-1 to T-239; M-1 to H-238; M-1 to L-237; M-1 to E-236; M-1 to L-235; M-1 to Q-234; M-1 to P-233; M-1 to E-232; M-1 to G-231; M-1 to L-230; M-1 to G-229; M-1 to A-228; M-1 to P-227; M-1 to A-226; M-1 to G-225; M-1 to Q-224; M-1 to S-223; M-1 to A-222; M-1 to F-221; M-1 to R-220; M-1 to V-219; M-1 to L-218; M-1 to K-217; M-1 to 1-216; M-1 to A-215; M-1 to G-214; M-1 to S-213; M-1 to A-212; M-1 to L-211; M-1 to P-210; M-1 to G-209; M-1 to L-208; M-1 to H-207; M-1 to E-206; M-1 to R-205; M-1 to Q-204; M-1 to V-203; M-1 to S-202; M-1 to V-201; M-1 to Q-200; M-1 to L-199; M-1 to L-198; M-1 to L-197; M-1 to P-196; M-1 to Q-195; M-1 to R-194; M-1 to P-193; M-1 to R-192; M-1 to S-191; M-1 to L-190; M-1 to Q-189; M-1 to Q-188; M-1 to W-187; M-1 to F-186; M-1 to N-185; M-1 to V-184; M-1 to A-183; M-1 to E-182; M-1 to T-181; M-1 to V-180; M-1 to D-179; M-1 to F-178; M-1 to A-177; M-1 to K-176; M-1 to W-175; M-1 to G-174; M-1 to S-173; M-1 to E-172; M-1 to H-171; M-1 to V-170; M-1 to S-169; M-1 to V-168; M-1 to L-167; M-1 to R-166; M-1 to S-165; M-1 to D-164; M-1 to I-163; M-1 to L-162; M-1 to S-161; M-1 to T-160; M-1 to R-159; M-1 to N-158; M-1 to S-157; M-1 to G-156; M-1 to D-155; M-1 to D-154; M-1 to R-153; M-1 to V-152; M-1 to R-151; M-1 to L-150; M-1 to W-149; M-1 to E-148; M-1 to V-147; M-1 to T-146; M-1 to V-145; M-1 to R-144; M-1 to A-143; M-1 to R-142; M-1 to A-141; M-1 to S-140; M-1 to R-139; to P-138; M-1 to S-137; M-1 to L-136; M-1 to R-135; M-1 to G-134; M-1 to H-133; M-1 to R-132; M-1 to H-131; M-1 to L-130; M-1 to A-129; M-1 to A-128; M-1 to K-127; M-1 to P-126; M-1 to V-125; M-1 to P-124; M-1 to E-123; M-1 to Q-122; M-1 to F-121; M-1 to L-120; M-1 to R-119; M-1 to L-118; M-1 to V-117; M-1 to A-116; M-1 to Q-115; M-1 to V-1 14; M-1 to L-113; M-1 to E-112; M-1 to S-111; M-1 to N-110; M-1 to P-109; M-1 to P-108; M-1 to L-107; M-1 to R-106; M-1 to Q-105; M-1 to E-104; M-1 to M-103; M-1 to G-102; M-1 to F-101; M-1 to V-100; M-1 to L-99; M-1 to L-98; M-1 to H-97; M-1 to T-96; M-1 to S-95; M-1 to A-94; M-1 to E-93; M-1 to L-92; M-1 to A-91; M-1 L-90 M-1 to F-89; M-1 to R-88; M-1 to G-87; M-1 to A-86; M-1 to V-85; M-1 to E-84; M-1 to R-83; M-1 to F-82; M-1 to S-81; M-1 to Q-80; M-1 to S-79; M-1 to F-78; M-1 to R-77; M-1 to K-76; M-1 to G-75; M-1 to R-74; M-1 to S-73; M-1 to R-72; M-1 to D-71; M-1 to G-70; M-1 to H-69; M-1 to S-68; M-1 to R-67; M-1 to Q-66; M-1 to L-65; M-1 to L-64; M-1 to A-63; M-1 to V-62; M-1 to Y-61; M-1 to Q-60; M-1 to A-59; M-1 to R-58; M-1 to V-57; M-1 to H-56; M-1 to T-55; M-1 to P-54; M-1 to 1-53; M-1 to V-52; M-1 to L-51; M-1 to E-50; M-1 to E-49; M-1 to M-48; M-1 to D-47; M-1 to A-46; M-1 to R-45; M-1 to D-44; M-1 to L-43; M-1 to T-42; M-1 to P-41; M-1 to V-40; M-1 to E-39; M-1 to K-38; M-1 to L-37; M-1 to Q-36; M-1 to L-35; M-1 to Q-34; M-1 to R-33; M-1 to L-32; M-1 to L-31; M-1 to S-30; M-1 to G-29; M-1 to L-28; M-1 to L-27; M-1 to Q-26; M-1 to E-25; M-1 to G-24; M-1 to T-23; M-1 to L-22; M-1 to A-21; M-1 to A-20; M-1 to G-19; M-1 to P-18; M-1 to S-17; M-1 to A-16; M-1 to L-15; M-1 to P-14; M-1 to L-13; M-1 to V-12; M-1 to W-11; M-1 to L-10; M-1 to A-9; M-1 to W-8; M-1 to C-7; and M-1 to L-6 of the sequence of the Human Lefty sequence shown in FIGS. 2A and B (which is identical to the sequence shown as SEQ ID NO:4, with the exception that the amino acid residues in FIGS. 2A and B are numbered consecutively from 1 through 366 from the N-terminus to the C-terminus, while the amino acid residues in SEQ ID NO:4 are numbered consecutively from −18 through 348 to reflect the position of the predicted signal peptide). Polynucleotides encoding these polypeptides also are provided.

The invention also provides polypeptides having one or more amino acids deleted from both the amino and the carboxyl termini of a Human Lefty polypeptide, which may be described generally as having residues n⁴-m⁴ of FIGS. 2A and B (SEQ ID NO:4), where n⁴ and m⁴ are integers as described above.

In addition to terminal deletion forms of the proteins discussed above, it also will be recognized by one of ordinary skill in the art that some amino acid sequences of the Nodal and Lefty polypeptides can be varied without significant effect of the structure or function of the proteins. If such differences in sequence are contemplated, it should be remembered that there will be critical areas on the protein which determine activity.

Thus, the invention further includes variations of the Nodal and Lefty polypeptides which show substantial Nodal or Lefty polypeptide activity or which include regions of Nodal or Lefty proteins such as the protein portions discussed below. Such mutants include deletions, insertions, inversions, repeats, and type substitutions selected according to general rules known in the art so as have little effect on activity. For example, guidance concerning how to make phenotypically silent amino acid substitutions is provided wherein the authors indicate that there are two main approaches for studying the tolerance of an amino acid sequence to change (Bowie, J. U., et al., Science 247:1306-1310 (1990)),. The first method relies on the process of evolution, in which mutations are either accepted or rejected by natural selection. The second approach uses genetic engineering to introduce amino acid changes at specific positions of a cloned gene and selections or screens to identify sequences that maintain functionality.

As the authors state, these studies have revealed that proteins are surprisingly tolerant of amino acid substitutions. The authors further indicate which amino acid changes are likely to be permissive at a certain position of the protein. For example, most buried amino acid residues require nonpolar side chains, whereas few features of surface side chains are generally conserved. Other such phenotypically silent substitutions are described by Bowie and coworkers (supra) and the references cited therein. Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr.

Thus, the fragment, derivative or analog of the polypeptides of SEQ ID NO:2 or SEQ ID NO:4, or those encoded by the deposited cDNAs, may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the active form of the polypeptide is fused with another compound, such as a compound to increase the half-life of the polypeptide (for example, polyethylene glycol), or (iv) one in which the additional amino acids are fused to the above form of the polypeptide, such as an IgG Fc fusion region peptide or leader or secretory sequence or a sequence which is employed for purification of the above form of the polypeptide or a proprotein sequence. Such fragments, derivatives and analogs are deemed to be within the scope of those skilled in the art from the teachings herein.

Thus, the Nodal and Lefty proteins of the present invention may include one or more amino acid substitutions, deletions or additions, either from natural mutations or human manipulation. As indicated, changes are preferably of a minor nature, such as conservative amino acid substitutions that do not significantly affect the folding or activity of the protein (see Table III).

TABLE III
Conservative Amino Acid Substitutions.

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

Embodiments of the invention are directed to polypeptides which comprise the amino acid sequence of a Nodal or Lefty polypeptide described herein, but having an amino acid sequence which contains at least one conservative amino acid substitution, but not more than 50 conservative amino acid substitutions, even more preferably, not more than 40 conservative amino acid substitutions, still more preferably, not more than 30 conservative amino acid substitutions, and still even more preferably, not more than 20 conservative amino acid substitutions, when compared with the Nodal or Lefty polynucleotide sequence described herein. Of course, in order of ever-increasing preference, it is highly preferable for a peptide or polypeptide to have an amino acid sequence which comprises the amino acid sequence of a Nodal or Lefty polypeptide, which contains at least one, but not more than 10, 9, 8, 7, 6, 5, 4, 3, 2 or 1 conservative amino acid substitutions.

In further specific embodiments, the number of substitutions, additions or deletions in the amino acid sequence of FIGS. 1A and B (SEQ ID NO:2), FIGS. 2A and B (SEQ ID NO:4), a polypeptide sequence encoded by the deposited clones, and/or any of the polypeptide fragments described herein (e.g., the mature forms or the active TGF-β consensus cleavage domains) is 75, 70, 60, 50, 40, 35, 30, 25, 20, 15, 10, 9, 8, 7, 6, 5, 4, 3, 2, 1 or 150-50, 100-50, 50-20, 30-20, 20-15, 20-10, 15-10, 10-1, 5-10, 1-5, 1-or 1-2.

To improve or alter the characteristics of Nodal or Lefty polypeptides, protein engineering may be employed. Recombinant DNA technology known to those skilled in the art can be used to create novel mutant polypeptides or muteins including single or multiple amino acid substitutions, deletions, additions or fusion proteins. Such modified polypeptides can show, e.g., enhanced activity or increased stability. In addition, they may be purified in higher yields and show better solubility than the corresponding natural polypeptide, at least under certain purification and storage conditions.

Thus, the invention also encompasses Nodal and Lefty derivatives and analogs that have one or more amino acid residues deleted, added, or substituted to generate Nodal and Lefty polypeptides that are better suited for expression, scale up, etc., in the host cells chosen. For example, cysteine residues can be deleted or substituted with another amino acid residue in order to eliminate disulfide bridges; N-linked glycosylation sites can be altered or eliminated to achieve, for example, expression of a homogeneous product that is more easily recovered and purified from yeast hosts which are known to hyperglycosylate N-linked sites. To this end, a variety of amino acid substitutions at one or both of the first or third amino acid positions on any one or more of the glycosylation recognition sequences in the Nodal and Lefty polypeptides of the invention, and/or an amino acid deletion at the second position of any one or more such recognition sequences will prevent glycosylation of the Nodal or Lefty polypeptide at the modified tripeptide sequence (see, e.g., Miyajima, A., et al., EMBO J. 5(6):1193-1197 (1986)).

Amino acids in the Nodal and Lefty polypeptides of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as receptor binding or in vitro proliferative activity.

Of special interest are substitutions of charged amino acids with other charged or neutral amino acids which may produce proteins with highly desirable improved characteristics, such as less aggregation. Aggregation may not only reduce activity but also be problematic when preparing pharmaceutical formulations, because aggregates can be immunogenic (Pinckard, et al., Clin. Exp. Immunol. 2:331-340 (1967); Robbins, et al., Diabetes 36:838-845 (1987); Cleland, et al., Crit. Rev. Therapeutic Drug Carrier Systems 10:307-377 (1993)).

Replacement of amino acids can also change the selectivity of the binding of a ligand to cell surface receptors (for example, Ostade, et al., Nature 361:266-268 (1993)) describes certain mutations resulting in selective binding of TNF-α to only one of the two known types of TNF receptors. Sites that are critical for ligand-receptor binding can also be determined by structural analysis such as crystallization, nuclear magnetic resonance or photoaffinity labeling (Smith, et al., J. Mol. Biol. 224:899-904 (1992); de Vos, et al. Science 255:306-312 (1992)).

Since Nodal and Lefty are members of the TGF-β-related protein family, to modulate rather than completely eliminate biological activities of Nodal and Lefty preferably mutations are made in sequences encoding amino acids in the Nodal and Lefty conserved domain, i.e., in positions 173 to 283 or SEQ ID NO:2 or positions 125 to 348 of SEQ ID NO:4, more preferably in residues within this region which are not conserved in all members of the TGF-β-related protein family. In particular, mutations to the Nodal and Lefty polypeptides are mad in positions other than the conserved cysteine residues comprising the “cysteine knot” motif characteristic of TGF-β-related protein family members. Also forming part of the present invention are isolated polynucleotides comprising nucleic acid sequences which encode the above Nodal and Lefty mutants.

The polypeptides of the present invention are preferably provided in an isolated form, and preferably are substantially purified. Recombinantly produced versions of the Nodal and Lefty polypeptides can be substantially purified by the one-step method described by Smith and Johnson (Gene 67:31-40 (1988)). Polypeptides of the invention also can be purified from natural or recombinant sources using anti-Nodal or anti-Lefty antibodies of the invention in methods which are well known in the art of protein purification.

The invention farther provides isolated Nodal and Lefty polypeptides comprising an amino acid sequence selected from the group consisting of: (a) the amino acid sequence of the full-length Nodal polypeptide having the complete amino acid sequence shown in SEQ ID NO:2 (i.e., positions 1 to 283 of SEQ ID NO:2); (b) the amino acid sequence of the predicted active Nodal polypeptide having the amino acid sequence at positions 173 to 283 of SEQ ID NO:2; (c) the amino acid sequence of the Nodal polypeptide having the complete amino acid sequence encoded by the cDNA clone contained in ATCC Deposit No. 209092 and/or 209135; (d) the amino acid sequence of the active domain of the Nodal polypeptide having the amino acid sequence encoded by the cDNA clone contained in ATCC Deposit No. 209092 and/or 209135; (e) the amino acid sequence of the Lefty polypeptide having the complete amino acid sequence in SEQ ID NO:4 (i.e., positions −18 to 348 of SEQ ID NO:4); (f) the amino acid sequence of the Lefty polypeptide having the complete amino acid sequence in SEQ ID NO:4 excepting the N-terminal methionine (i.e., positions −17 to 348 of SEQ ID NO:4); (g) the amino acid sequence of the predicted active domain of the Lefty polypeptide having the amino acid sequence at positions 60 to 348 of SEQ ID NO:4; (h) the amino acid sequence of the predicted active domain of the Lefty polypeptide having the amino acid sequence at positions 118 to 348 of SEQ ID NO:4; (i) the amino acid sequence of the predicted active domain of the Lefty polypeptide having the amino acid sequence at positions 125 to 348 of SEQ ID NO:4; (j) the amino acid sequence of the Lefty polypeptide having the complete amino acid sequence encoded by the cDNA clone contained in ATCC Deposit No. 209091; (k) the amino acid sequence of the Lefty polypeptide having the complete amino acid sequence excepting the N-terminal methionine encoded by the cDNA clone contained in ATCC Deposit No. 209091, and; (1) the amino acid sequence of the active domain of the Lefty polypeptide having the amino acid sequence encoded by the cDNA clone contained in ATCC Deposit No. 209091.

Further polypeptides of the present invention include polypeptides which have at least 90% similarity, more preferably at least 95% similarity, and still more preferably at least 96%, 97%, 98% or 99% similarity to those described above. The polypeptides of the invention also comprise those which are at least 80% identical, more preferably at least 90% or 95% identical, still more preferably at least 96%, 97%, 98% or 99% identical to the polypeptide encoded by the deposited cDNAs or to the polypeptides of SEQ ID NO:2 or SEQ ID NO:4, and also include portions of such polypeptides with at least 30 amino acids and more preferably at least 50 amino acids.

By “% similarity” for two polypeptides is intended a similarity score produced by comparing the amino acid sequences of the two polypeptides using the Bestfit program (Wisconsin Sequence Analysis Package, Version 8 for Unix, Genetics Computer Group, University Research Park, 575 Science Drive, Madison, Wis. 53711) and the default settings for determining similarity. Bestfit uses the local homology algorithm of Smith and Waterman (Advances in Applied Mathematics 2:482-489, 1981) to find the best segment of similarity between two sequences.

By a polypeptide having an amino acid sequence at least, for example, 95% “identical” to a reference amino acid sequence of a Nodal or Lefty polypeptide is intended that the amino acid sequence of the polypeptide is identical to the reference sequence except that the polypeptide sequence may include up to five amino acid alterations per each 100 amino acids of the reference amino acid of the Nodal or Lefty polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to, for instance, the amino acid sequence shown in FIGS. 1A and B (SEQ ID NO:2), the amino acid sequence shown in FIGS. 2A and B (SEQ ID NO:4), the amino acid sequence encoded by deposited cDNA clones HTLFA20, HNGEF08, and HUKEJ46, or fragments thereof, can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

In a specific embodiment, the identity between a reference (query) sequence (a sequence of the present invention) and a subject sequence, also referred to as a global sequence alignment, is determined using the FASTDB computer program based on the algorithm of Brutlag et al. (Comp. App. Biosci. 6:237-245 (1990)). Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty0.05, Window Size=500 or the length of the subject amino acid sequence, whichever is shorter. According to this embodiment, if the subject sequence is shorter than the query sequence due to N- or C-terminal deletions, not because of internal deletions, a manual correction is made to the results to take into consideration the fact that the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. A determination of whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of this embodiment. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence. For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. No other manual corrections are made for the purposes of this embodiment.

The invention also encompasses fusion proteins in which the full-length Nodal or Lefty polypeptide or fragment, variant, derivative, or analog thereof is fused to an unrelated protein. These fusion proteins can be routinely designed on the basis of the Nodal or Lefty nucleotide and polypeptide sequences disclosed herein. For example, as one of skill in the art will appreciate, Nodal and/or Lefty polypeptides and fragments (including epitope-bearing fragments) thereof described herein can be combined with parts of the constant domain of immunoglobulins (IgG), resulting in chimeric (fusion) polypeptides. These fusion proteins facilitate purification and show an increased half-life in vivo. This has been shown, e.g., for chimeric proteins consisting of the first two domains of the human CD4-polypeptide and various domains of the constant regions of the heavy or light chains of mammalian immunoglobulins (EP A 394,827; Traunecker, et al., Nature 331:84-86 (1988)). Fusion proteins that have a disulfide-linked dimeric structure due to the IgG part can also be more efficient in binding and neutralizing other molecules than the monomeric Nodal or Lefty proteins or protein fragments alone (Fountoulakis, et al., J. Biochem. 270:3958-3964 (1995)). Examples of Nodal and Lefty fusion proteins that are encompassed by the invention include, but are not limited to, fusion of the Nodal or Lefty polypeptide sequences to any amino acid sequence that allows the fusion proteins to be displayed on the cell surface (e.g. the IgG Fc domain); or fusions to an enzyme, fluorescent protein, or luminescent protein which provides a marker function.

Antibodies

Nodal or Lefty polypeptide-specific antibodies for use in the present invention can be raised against the intact Nodal or Lefty protein or an antigenic polypeptide fragment thereof, which may be presented together with a carrier protein, such as an albumin, to an animal system (such as rabbit or mouse) or, if it is long enough (at least about 25 amino acids), without a carrier.

As used herein, the term “antibody” (Ab) or “monoclonal antibody” (Nab) is meant to include intact molecules as well as antibody fragments (such as, for example, Fab and F(ab′)2 fragments) which are capable of specifically binding to Nodal or Lefty protein. Fab and F(ab′)2 fragments lack the Fc fragment of intact antibody, clear more rapidly from the circulation, and may have less non-specific tissue binding of an intact antibody (Wahl, et al., J. Nucl. Med. 24:316-325 (1983)). Thus, these fragments are preferred.

The antibodies of the present invention may be prepared by any of a variety of methods. For example, cells expressing the Nodal or Lefty protein or an antigenic fragment thereof can be administered to an animal in order to induce the production of sera containing polyclonal antibodies. In a preferred method, a preparation of Nodal and Lefty protein is prepared and purified to render it substantially free of natural contaminants. Such a preparation is then introduced into an animal in order to produce polyclonal antisera of greater specific activity.

In the most preferred method, the antibodies of the present invention are monoclonal antibodies (or Nodal or Lefty protein binding fragments thereof). Such monoclonal antibodies can be prepared using hybridoma technology (Kohler, et al., Nature 256:495 (1975); Kohler, et al., Eur. J.Immunol. 6:511 (1976); Kohler, et al., Eur. J Immunol. 6:292 (1976); Hammerling, et al., in: Monoclonal Antibodies and T-Cell Hybridomas, Elsevier, N.Y., (1981) pp. 563-681)). In general, such procedures involve immunizing an animal (preferably a mouse) with a Nodal or Lefty protein antigen or, more preferably, with a Nodal or Lefty protein-expressing cell. Suitable cells can be recognized by their capacity to bind anti-Nodal or anti-Lefty protein antibody. Such cells may be cultured in any suitable tissue culture medium; however, it is preferable to culture cells in Earle's modified Eagle's medium supplemented with 10% fetal bovine serum (inactivated at about 56° C.), and supplemented with about 10 μg/ml of nonessential amino acids, about 1,000 U/ml of penicillin, and about 100 μg/ml of streptomycin. The splenocytes of such mice are extracted and fused with a suitable myeloma cell line. Any suitable myeloma cell line may be employed in accordance with the present invention; however, it is preferable to employ the parent myeloma cell line (SP2O), available from the American Type Culture Collection, Rockville, Md. After fusion, the resulting hybridoma cells are selectively maintained in HAT medium, and then cloned by limiting dilution as described by Wands and colleagues (Gastroenterology 80:225-232 (1981)). The hybridoma cells obtained through such a selection are then assayed to identify clones which secrete antibodies capable of binding the Nodal or Lefty protein antigen.

Alternatively, additional antibodies capable of binding to the Nodal or Lefty protein antigens may be produced in a two-step procedure through the use of anti-idiotypic antibodies. Such a method makes use of the fact that antibodies are themselves antigens, and that, therefore, it is possible to obtain an antibody which binds to a second antibody. In accordance with this method, Nodal or Lefty protein-specific antibodies are used to immunize an animal, preferably a mouse. The splenocytes of such an animal are then used to produce hybridoma cells, and the hybridoma cells are screened to identify clones which produce an antibody whose ability to bind to the Nodal or Lefty protein-specific antibody can be blocked by the Nodal or Lefty protein antigen. Such antibodies comprise anti-idiotypic antibodies to the Nodal or Lefty protein-specific antibodies and can be used to immunize an animal to induce formation of further Nodal or Lefty protein-specific antibodies.

It will be appreciated that Fab and F(ab′)2 and other fragments of the antibodies of the present invention may be used according to the methods disclosed herein. Such fragments are typically produced by proteolytic cleavage, using enzymes such as papain (to produce Fab fragments) or pepsin (to produce F(ab′)2 fragments). Alternatively, Nodal or Lefty protein-binding fragments can be produced through the application of recombinant DNA technology or through synthetic chemistry.

For in vivo use of anti-Nodal and anti-Lefty in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art (Morrison, Science 229:1202 (1985); Oi, et al., BioTechniques 4:214 (1986); Cabilly, et al., U.S. Pat. No. 4,816,567; Taniguchi, et al., EP 171496; Morrison, et al., EP 173494; Neuberger, et al., WO 8601533; Robinson, et al., WO 8702671; Boulianne, et al., Nature 312:643 (1984); Neuberger, et al., Nature 314:268 (1985).

### CELLULAR GROWTH AND DIFFERENTIATION-RELATED DISORDERS

Diagnosis

The present inventors have discovered that Nodal is expressed in neutrophils and testes. In addition, the present inventors have discovered that Lefty is expressed in uterine cancer, colon cancer, apoptotic T-cells, fetal heart, Wilm's Tumor tissue, frontal lobe of the brain from a patient with dementia, neutrophils, salivary gland, small intestine, 7, 8, and 12 week old human embryos, frontal cortex and hypothalamus from a patient with schizophrenia, brain from a patient with Alzheimer's Disease, adipose tissue, brown fat, TNF- and LPS-induced and uninduced bone marrow stroma, activated monocytes and macrophages, rhabdomyosarcoma, cycloheximide-treated Raji cells, breast lymph nodes, hemangiopericytoma, testes, fetal epithelium (skin), and IL-5-induced eosinophils.. For a number of cell growth and differentiation-related disorders, substantially altered (increased or decreased) levels of Nodal or Lefty gene expression can be detected in affected tissues, cells, or bodily fluids (e.g., sera, plasma, urine, synovial fluid or spinal fluid) taken from an individual having such a disorder, relative to a “standard” Nodal or Lefty gene expression level, that is, the Nodal and Lefty expression level in affected tissues or bodily fluids from an individual not having the cell growth and differentiation disorder. Thus, the invention provides a diagnostic method useful during diagnosis of a cell growth and differentiation disorder, which involves measuring the expression level of the gene encoding the Nodal or Lefty proteins in affected tissues, cells, or body fluids from an individual and comparing the measured gene expression level with a standard Nodal or Lefty gene expression level, whereby an increase or decrease in the gene expression level compared to the standard is indicative of a cell growth and differentiation disorder.

In particular, it is believed that certain tissues in mammals with cancer of the immune or reproductive systems express significantly reduced levels of the Nodal or Lefty proteins and mRNA encoding the Nodal or Lefty proteins when compared to corresponding “standard” levels. Further, it is believed that enhanced levels of the Nodal or Lefty proteins can be detected in certain body fluids (e.g., sera, plasma, urine, and spinal fluid) from mammals with such a cancer when compared to sera from mammals of the same species not having the cancer.

Thus, the invention provides a diagnostic method useful during diagnosis of a cellular growth and differentiation disorder, including cancers, which involves measuring the expression level of the genes encoding the Nodal and Lefty proteins in tissues, cells, or body fluids from an individual and comparing the measured gene expression levels with standard Nodal and Lefty gene expression levels, whereby an increase or decrease in the gene expression level compared to the standard is indicative of a cell growth and differentiation disorder.

Where a diagnosis of a disorder in the regulation of cell growth and differentiation, including diagnosis of a tumor, has already been made according to conventional methods, the present invention is useful as a prognostic indicator, whereby patients exhibiting depressed Nodal or Lefty gene expression will experience a worse clinical outcome relative to patients expressing the gene at a level nearer the standard level.

By “assaying the expression level of the genes encoding the Nodal and Lefty polypeptides” is intended qualitatively or quantitatively measuring or estimating the level of the Nodal and Lefty polypeptides or the level of the mRNA encoding the Nodal and Lefty polypeptides in a first biological sample either directly (e.g., by determining or estimating absolute protein level or mRNA level) or relatively (e.g., by comparing to the Nodal and Lefty polypeptides levels or mRNA level in a second biological sample). Preferably, the Nodal and Lefty polypeptides levels or mRNA levels in the first biological sample is measured or estimated and compared to a standard Nodal and Lefty polypeptide level or mRNA level, the standard being taken from a second biological sample obtained from an individual not having the disorder or being determined by averaging levels from a population of individuals not having a disorder of cellular growth and differentiation. As will be appreciated in the art, once standard Nodal and Lefty polypeptides levels or mRNA levels are known, they can be used repeatedly as a standard for comparison.

By “biological sample” is intended any biological sample obtained from an individual, body fluid, cell line, tissue culture, or other source which contains Nodal and Lefty protein or mRNA. As indicated, biological samples include body fluids (such as sera, plasma, urine, synovial fluid and spinal fluid) which contain free active forms of Nodal or Lefty protein, tissues exhibiting the effects of abnormally regulated cell growth or differentiation, and other tissue sources found to express complete, mature, or active forms of the Nodal or Lefty proteins or a Nodal or Lefty receptor. Methods for obtaining tissue biopsies and body fluids from mammals are well known in the art. Where the biological sample is to include mRNA, a tissue biopsy is the preferred source.

The present invention is useful for diagnosis or treatment of various cell growth and differentiation-related disorders in mammals, preferably humans. Such disorders include tumors, cancers, interstitial lung disease, and any disregulation of the growth and differentiation patterns of cell function including, but not limited to, autoimmunity, arthritis, leukemias, lymphomas, immunosuppression, immunity, humoral immunity, inflammatory bowel disease, myelosuppression, and the like.

Total cellular RNA can be isolated from a biological sample using any suitable technique such as the single-step guanidinium-thiocyanate-phenol-chloroform method described by Chomczynski and Sacchi (Anal. Biochem. 162:156-159 (1987)). Levels of mRNA encoding the Nodal and Lefty polypeptides are then assayed using any appropriate method. These include Northern blot analysis, S 1 nuclease mapping, the polymerase chain reaction (PCR), reverse transcription in combination with the polymerase chain reaction reaction (RT-PCR), and reverse transcription in combination with the ligase chain reaction (RT-LCR).

Assaying Nodal and Lefty polypeptides levels in a biological sample can occur using antibody-based techniques. For example, Nodal and Lefty protein expression in tissues can be studied with classical immunohistological methods (Jalkanen, M., et al., J. Cell. Biol. 101:976-985 (1985); Jalkanen, M., et al., J. Cell. Biol. 105:3087-3096 (1987)). Other antibody-based methods useful for detecting Nodal and Lefty polypeptides gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes, such as iodine (¹²⁵I, ¹²¹I), carbon (¹⁴C), sulfur (³⁵S), tritium (³H), indium (¹¹²In), and technetium (^{99m}Tc), and fluorescent labels, such as fluorescein and rhodamine, and biotin.

In addition to assaying Nodal and Lefty protein levels in a biological sample obtained from an individual, Nodal and Lefty polypeptides can also be detected in vivo by imaging. Antibody labels or markers for in vivo imaging of Nodal or Lefty protein include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma.

A Nodal or Lefty polypeptide-specific antibody or antibody fragment which has been labeled with an appropriate detectable imaging moiety, such as a radioisotope (for example, ¹³¹I, ¹¹²In, ^{99m}Tc), a radio-opaque substance, or a material detectable by nuclear magnetic resonance, is introduced (for example, parenterally, subcutaneously or intraperitoneally) into the mammal to be examined for immune system disorder. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ^{99m}Tc. The labeled antibody or antibody fragment will then preferentially accumulate at the location of cells which contain Nodal and Lefty protein. in vivo tumor imaging is described by Burchiel and coworkers (Chapter 13 in Tumor Imaging: The Radiochemical Detection of Cancer, Burchiel, S. W. and Rhodes, B. A., eds., Masson Publishing Inc. (1982)).

Treatment

As noted above, Nodal and Lefty polynucleotides and polypeptides are useful for diagnosis of conditions involving abnormally high or low expression of Nodal and Lefty activities. Given the cells and tissues where Nodal and Lefty are expressed as well as the activities modulated by Nodal and Lefty, it is readily apparent that a substantially altered (increased or decreased) level of expression of Nodal and Lefty in an individual compared to the standard or “normal” level produces pathological conditions related to the bodily system(s) in which Nodal and Lefty are expressed and/or are active.

It will also be appreciated by one of ordinary skill that, since the Nodal and Lefty proteins of the invention are members of the TGF-β superfamily the active domains of the proteins may be released in soluble form from the cells which express the Nodal and Lefty by proteolytic cleavage. Therefore, when Nodal or Lefty active domain is added from an exogenous source to cells, tissues or the body of an individual, the protein will exert its physiological activities on its target cells of that individual.

Therefore, it will be appreciated that conditions caused by a decrease in the standard or normal level of Nodal or Lefty activity in an individual, particularly disorders of cell growth and differentiation, can be treated by administration of the active form of Nodal or Lefty polypeptides. Thus, the invention also provides a method of treatment of an individual in need of an increased level of Nodal or Lefty activity comprising administering to such an individual a pharmaceutical composition comprising an amount of an isolated Nodal or Lefty polypeptide of the invention, particularly the active form of the Nodal and Lefty protein of the invention, effective to increase the Nodal and Lefty activity level in such an individual.

Since Nodal and Lefty inhibit endothelial cell function, compositions (e.g., polynucleotides, polypeptides, and fragments variants, derivatives and analogs thereof, and antibodies thereto, and agonists and antagonists thereto) corresponding to these genes may be used as anti-inflammatories. Nodal and Lefty compositions may also be employed to inhibit T-cell proliferation by the inhibition of IL-2 biosynthesis for the treatment of T-cell mediated auto-immune diseases and lymphocytic leukemias. In addition, compositions corresponding to Nodal and Lefty regulate T_{H1}/T_{H2} cytokine production. Further, Nodal and Lefty compositions may also be administered to treat or prevent inflammation, allergy, and infectious diseases or as an adjuvant for immunotherapy of tumors. Nodal and Lefty compositions may also be employed to stimulate wound healing. In this same manner, Nodal and Lefty compounds may also be employed to regulate hematopoiesis, by regulating the activation and differentiation of various hematopoietic progenitor cells, such as for example, to stimulate erythropoiesis or to stimulate the release of mature leukocytes from the bone marrow following chemotherapy, i.e., in stem cell mobilization.

Since Nodal is essential for mesoderm formation and subsequent organization of axial structures in early mouse development, the human Nodal homologue of the present invention is also likely involved developmental processes such as the correct formation of various structures or in one or more post-developmental capacities including sexual development, pituitary hormone production, and the creation of bone and cartilage, as are many of the other members of the TGF-β superfamily. Accordingly, the invention encompasses the use of Nodal compositions to regulate these processes, such as, for example, in stimulating bone and/or cartilage formation, and stimulating the production of pituitary hormone.

Since murine Lefty is important in left/right handedness of the developing organism. The homology between murine Lefty and the novel human Lefty homologue of the present invention indicates that the novel human Lefty homologue of the present invention may also be involved in correct formation of various structures with respect to the rest of the developing organism or Lefty may also be involved in one or more post-developmental capacities including sexual development, pituitary hormone production, and the creation of bone and cartilage, as are many of the other members of the TGF-β superfamily. Accordingly, the invention encompasses the use of Nodal compositions to regulate these processes, such as, for example, in stimulating bone and/or cartilage formation, and stimulating the production of hormones in the pituitary.

Nodal and Lefty compounds may also be administered regulate or modulate cell growth and differentiation which is not necessarily associated with endogenously high or low levels of Nodal and/or Lefty. For example, Nodal and Lefty polypeptides of the present invention are useful for enhancing or enriching the growth and/or differentiation of specific cell populations, e.g., embryonic cells or stem cells.

Formulations and Administration

The Nodal and/or Lefty polypeptide composition will be formulated and dosed in a fashion consistent with good medical practice, taking into account the clinical condition of the individual patient (especially the side effects of treatment with Nodal and/or Lefty polypeptide alone), the site of delivery of the Nodal and/or Lefty polypeptide composition, the method of administration, the scheduling of administration, and other factors known to practitioners. The “effective amount” of Nodal and/or Lefty polypeptide for purposes herein is thus determined by such considerations.

As a general proposition, the total pharmaceutically effective amount of Nodal and/or Lefty polypeptide administered parenterally per dose will be in the range of about 1 μg/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the hormone. If given continuously, the Nodal and/or Lefty polypeptide is typically administered at a dose rate of about 1 μg/kg/hour to about 50 μg/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Pharmaceutical compositions containing the Nodal and Lefty proteins of the invention may be administered orally, rectally, parenterally, intracistemally, intravaginally, intraperitoneally, topically (as by powders, ointments, drops or transdermal patch), bucally, or as an oral or nasal spray. By “pharmaceutically acceptable carrier” is meant a non-toxic solid, semisolid or liquid filler, diluent, encapsulating material or formulation auxiliary of any type. The term “parenteral” as used herein refers to modes of administration which include intravenous, intramuscular, intraperitoneal, intrastemal, subcutaneous and intraarticular injection and infusion.

The Nodal and Lefty polypeptides are also suitably administered by sustained-release systems. Suitable examples of sustained-release compositions include semi-permeable polymer matrices in the form of shaped articles, e.g., films, or microcapsules. Sustained-release matrices include polylactides (U.S. Pat. No. 3,773,919, EP 58,481), copolymers of L-glutamic acid and gamma-ethyl-L-glutamate (Sidman, U., et al., Biopolymers 22:547-556 (1983)), poly (2-hydroxyethyl methacrylate; Langer, R., et al, J. Biomed. Mater. Res. 15:167-277 (1981), and Langer, R., Chem. Tech. 12:98-105 (1982)), ethylene vinyl acetate (Langer, R., et al., Id.) or poly-D-(−)-3-hydroxybutyric acid (EP 133,988). Sustained-release Nodal and Lefty polypeptide compositions also include liposomally entrapped Nodal and Lefty polypeptides. Liposomes containing Nodal and Lefty polypeptides are prepared by methods known in the art (DE 3,218,121; Epstein, et al., Proc. Natl. Acad. Sci. (USA) 82:3688-3692 (1985); Hwang, et al., Proc. Natl. Acad. Sci. (USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324). Ordinarily, the liposomes are of the small (about 200-800 Angstroms) unilamellar type in which the lipid content is greater than about 30 mol. percent cholesterol, the selected proportion being adjusted for the optimal Nodal and Lefty polypeptide therapy.

For parenteral administration, in one embodiment, the Nodal and/or Lefty polypeptide is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides.

Generally, the formulations are prepared by contacting the Nodal and Lefty polypeptide uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium; and/or nonionic surfactants such as polysorbates, poloxamers, or PEG.

Another embodiment of the invention provides pharmaceutical compositions which contain a therapeutically effective amount of human Nodal and/or Lefty polypeptide, in a pharmaceutically acceptable vehicle or carrier. These compositions of the invention may be useful in the therapeutic modulation or diagnosis of bone, cartilage, or other connective cell or tissue growth and/or differentiation. These compositions may be used to treat such conditions as osteoarthritis, osteoporosis, and other abnormalities of bone, cartilage, muscle, tendon, ligament and/or other connective tissues and/or organs such as liver, lung, cardiac, pancreas, kidney, and other tissues. These compositions may also be useful in the growth and/or formation of cartilage, tendon, ligament, meniscus, and other connective tissues or any combination of the above (e.g., therapeutic modulation of the tendon-to-bone attachment apparatus). These compositions may also be useful in treating periodontal disease and modulating wound healing and tissue repair of such tissues as epidermis, nerve, muscle, cardiac muscle, liver, lung, cardiac, pancreas, kidney, and other tissues and/or organs. Pharmaceutical compositions containing Nodal and/or Lefty of the invention may include one or more other therapeutically useful component such as BMP-1, BMP-2, BMP-3, BMP-4, BMP-5, BMP-6, and/or BMP-7 (See, for example, U. S. Pat. Nos. 5,108,922; 5,013,649; 5,116,738; 5,106,748; 5,187,076; and 5,141,905), BMP-8 (See, for example, PCT publication WO91/18098), BMP-9 (See, for example, PCT publication WO93/00432), BMP-10 (See, for example, PCT publication WO94/26893), BMP-11 (See, for example, PCT publication WO94/26892), BMP-12 and/or BMP-13 (See, for example, PCT publication WO95/16035), with other growth factors including, but not limited to, BIP, one or more of the growth and differentiation factors (GDFs), VGR-2, epidermal growth factor (EGF), fibroblast growth factor (FGF), TGF-alpha, TGF-beta, activins, inhibins, and insulin-like growth factor (IGF).

The Nodal and Lefty polypeptides are typically formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of Nodal and Lefty polypeptide salts.

Nodal and Lefty polypeptides to be used for therapeutic administration must be sterile. Sterility is readily accomplished by filtration through sterile filtration membranes (e.g., 0.2 micron membranes). Therapeutic Nodal and Lefty polypeptide compositions generally are placed into a container having a sterile access port, for example, an intravenous solution bag or vial having a stopper pierceable by a hypodermic injection needle.

Nodal and Lefty polypeptides ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous Nodal and Lefty polypeptide solution, and the resulting mixture is lyophilized. The infusion solution is prepared by reconstituting the lyophilized Nodal and Lefty polypeptide using bacteriostatic water-for-injection (WFI).

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the polypeptides of the present invention may be employed in conjunction with other therapeutic compounds.

Agonists and Antagonists—Assays and Molecules

The invention also provides a method of screening compounds to identify those which enhance or block the action of Nodal and Lefty on cells, such as their interactions with Nodal- or Lefty-binding molecules such as receptor molecules. An agonist is a compound which increases the natural biological functions of Nodal or Lefty or which functions in a manner similar to Nodal or Lefty, while antagonists decrease or eliminate such functions.

In another embodiment, the invention provides a method for identifying a receptor protein or other ligand-binding protein which binds specifically to a Nodal or Lefty polypeptide. For example, a cellular compartment, such as a membrane or a preparation thereof, may be prepared from a cell that expresses a molecule that binds Nodal or Lefty. The preparation is incubated with labeled Nodal or Lefty and complexes of Nodal or Lefty bound to the receptor or other binding protein are isolated and characterized according to routine methods known in the art. Alternatively, the Nodal or Lefty polypeptides may be bound to a solid support so that binding molecules solubilized from cells are bound to the column and then eluted and characterized according to routine methods.

In the assay of the invention for agonists or antagonists, a cellular compartment, such as a membrane or a preparation thereof, may be prepared from a cell that expresses a molecule that binds Nodal or Lefty, such as a molecule of a signaling or regulatory pathway modulated by Nodal or Lefty. The preparation is incubated with labeled Nodal or Lefty in the absence or the presence of a candidate molecule which may be a Nodal or Lefty agonist or antagonist. The ability of the candidate molecule to bind the binding molecule is reflected in decreased binding of the labeled ligand. Molecules which bind gratuitously, i.e., without inducing the effects of Nodal or Lefty on binding the Nodal or Lefty binding molecule, are most likely to be good antagonists. Molecules that bind well and elicit effects that are the same as or closely related to Nodal or Lefty are agonists.

Nodal or Lefty-like effects of potential agonists and antagonists may by measured, for instance, by determining activity of a second messenger system following interaction of the candidate molecule with a cell or appropriate cell preparation, and comparing the effect with that of Nodal or Lefty or molecules that elicit the same effects as Nodal or Lefty. Second messenger systems that may be useful in this regard include but are not limited to AMP guanylate cyclase, ion channel or phosphoinositide hydrolysis second messenger systems.

Another example of an assay for Nodal and Lefty antagonists is a competitive assay that combines Nodal or Lefty and a potential antagonist with membrane-bound Nodal or Lefty receptor molecules or recombinant Nodal or Lefty receptor molecules under appropriate conditions for a competitive inhibition assay. Nodal and Lefty can be labeled, such as by radioactivity, such that the number of Nodal or Lefty molecules bound to a receptor molecule can be determined accurately to assess the effectiveness of the potential antagonist.

Potential antagonists include small organic molecules, peptides, polypeptides and antibodies that bind to a polypeptide of the invention and thereby inhibit or extinguish its activity. Potential antagonists also may be small organic molecules, a peptide, a polypeptide such as a closely related protein or antibody that binds the same sites on a binding molecule, such as a receptor molecule, without inducing Nodal- or Lefty-induced activities, thereby preventing the action of Nodal or Lefty by excluding Nodal or Lefty from binding.

Other potential antagonists include antisense molecules. Antisense technology can be used to control gene expression through antisense DNA or RNA or through triple-helix formation. Antisense techniques are discussed in a number of studies (for example, Okano, J. Neurochem. 56:560 (1991); “Oligodeoxynucleotides as Antisense Inhibitors of Gene Expression.” CRC Press, Boca Raton, Fla. (1988)). Triple helix formation is discussed in a number of studies, as well (for instance, Lee, et al., Nucleic Acids Research 6:3073 (1979); Cooney, et al., Science 241:456 (1988); Dervan, et al., Science 251:1360 (1991)). The methods are based on binding of a polynucleotide to a complementary DNA or RNA. For example, the 5′ coding portion of a polynucleotide that encodes the mature polypeptide of the present invention may be used to design an antisense RNA oligonucleotide of from about 10 to 40 base pairs in length. A DNA oligonucleotide is designed to be complementary to a region of the gene involved in transcription thereby preventing transcription and the production of Nodal or Lefty. The antisense RNA oligonucleotide hybridizes to the mRNA in vivo and blocks translation of the mRNA molecule into Nodal and Lefty polypeptide. The oligonucleotides described above can also be delivered to cells such that the antisense RNA or DNA may be expressed in vivo to inhibit production of Nodal or Lefty protein.

The agonists and antagonists may be employed in a composition with a pharmaceutically acceptable carrier, e.g., as described above.

The antagonists may be employed for instance to inhibit the activation of macrophages and their precursors, and of neutrophils, basophils, B lymphocytes and some T-cell subsets, e.g., activated and CD8 cytotoxic T cells and natural killer cells, in certain autoimmune and chronic inflammatory and infective diseases. Examples of autoimmune diseases include multiple sclerosis, and insulin-dependent diabetes. The antagonists may also be employed to treat infectious diseases including silicosis, sarcoidosis, idiopathic pulmonary fibrosis by preventing the recruitment and activation of mononuclear phagocytes. They may also be employed to treat idiopathic hyper-eosinophilic syndrome by preventing eosinophil production and stimulation. Endotoxic shock may also be treated by the antagonists by preventing the stimulation of macrophages and their production of the human chemokine polypeptides of the present invention. The antagonists may also be employed to treat histamine-mediated allergic reactions and immunological disorders including late phase allergic reactions, chronic urticaria, and atopic dermatitis by inhibiting mast cell and basophil degranulation and release of histamine. IgE-mediated allergic reactions such as allergic asthma, rhinitis, and eczema may also be treated. The antagonists may also be employed to treat chronic and acute inflammation by preventing the activation of monocytes in a wound area. Antagonists may also be employed to treat rheumatoid arthritis by preventing the activation of monocytes in the synovial fluid in the joints of patients. Monocyte activation plays a significant role in the pathogenesis of both degenerative and inflammatory arthropathies. The antagonists may be employed to interfere with the deleterious cascades attributed primarily to IL-1 and TNF, which prevents the biosynthesis of other inflammatory cytokines. In this way, the antagonists may be employed to prevent inflammation. The antagonists may also be employed to treat cases of bone marrow failure, for example, aplastic anemia and myelodysplastic syndrome. Any of the above antagonists may be employed in a composition with a pharmaceutically acceptable carrier, e.g., as hereinafter described.

Gene Mapping

The nucleic acid molecules of the present invention are also valuable for chromosome identification. The sequence is specifically targeted to and can hybridize with a particular location on an individual human chromosome. Moreover, there is a current need for identifying particular sites on the chromosome. Few chromosome marking reagents based on actual sequence data (repeat polymorphisms) are presently available for marking chromosomal location. The mapping of DNAs to chromosomes according to the present invention is an important first step in correlating those sequences with genes associated with disease.

In certain preferred embodiments in this regard, the cDNAs herein disclosed are used to clone genomic DNAs of Nodal and Lefty protein genes. This can be accomplished using a variety of well known techniques and libraries, which generally are available commercially. The genomic DNAs then are used for in situ chromosome mapping using well known techniques for this purpose.

In addition, in some cases, sequences can be mapped to chromosomes by preparing PCR primers (preferably 15-25 bp) from the cDNA. Computer analysis of the 3′ untranslated region of the gene is used to rapidly select primers that do not span more than one exon in the genomic DNA, thus complicating the amplification process. These primers are then used for PCR screening of somatic cell hybrids containing individual human chromosomes. Fluorescence in situ hybridization (“FISH”) of a cDNA clone to a metaphase chromosomal spread can be used to provide a precise chromosomal location in one step. This technique can be used with probes from the cDNA as short as 50 or 60 bp (for a review of this technique, see Verma, et al., Human Chromosomes: A Manual Of Basic Techniques, Pergamon Press, New York (1988)).

Once a sequence has been mapped to a precise chromosomal location, the physical position of the sequence on the chromosome can be correlated with genetic map data. Such data are found, for example, on the World Wide Web (McKusick, V. Mendelian Inheritance In Man, available on-line through Johns Hopkins University, Welch Medical Library). The relationship between genes and diseases that have been mapped to the same chromosomal region are then identified through linkage analysis (coinheritance of physically adjacent genes).

Next, it is necessary to determine the differences in the cDNA or genomic sequence between affected and unaffected individuals. If a mutation is observed in some or all of the affected individuals but not in any normal individuals, then the mutation is likely to be the causative agent of the disease.

Having generally described the invention, the same will be more readily understood by reference to the following examples, which are provided by way of illustration and are not intended as limiting.

### EXAMPLES

### Example 1(a)

### Expression and Purification of “His-tagged” Nodal in E. coli

The bacterial expression vector pQE9 (pD10) is used for bacterial expression in this example. (QIAGEN, Inc., 9259 Eton Avenue, Chatsworth, Calif., 91311). pQE9 encodes ampicillin antibiotic resistance (“Ampr”) and contains a bacterial origin of replication (“ori”), an IPTG inducible promoter, a ribosome binding site (“RBS”), six codons encoding histidine residues that allow affinity purification using nickel-nitrilo-tri-acetic acid (“Ni-NTA”) affinity resin sold by QIAGEN, Inc., supra, and suitable single restriction enzyme cleavage sites. These elements are arranged such that an inserted DNA fragment encoding a polypeptide expresses that polypeptide with the six His residues (i.e., a “6×His tag”) covalently linked to the amino terminus of that polypeptide.

The DNA sequence encoding the desired portion of the Nodal and Lefty protein comprising the active domain of the Nodal amino acid sequence is amplified from the deposited cDNA clone using PCR oligonucleotide primers which anneal to the amino terminal sequences of the desired portion of the Nodal and Lefty protein and to sequences in the deposited construct 3′ to the cDNA coding sequence. Additional nucleotides containing restriction sites to facilitate cloning in the pQE9 vector are added to the 5′ and 3′ primer sequences, respectively.

For cloning the active form of the Nodal protein, the 5′ primer has the sequence 5′ CGC GGA TCC CAT CAC TTG CCA GAC AGA AG 3′ (SEQ If) NO:9) containing the underlined Barn HI restriction site followed by 20 nucleotides of the amino terminal coding sequence of the mature Nodal sequence in SEQ ID NO:2. One of ordinary skill in the art would appreciate, of course, that the point in the protein coding sequence where the 5′ primer begins may be varied to amplify a DNA segment encoding any desired portion of the complete Nodal protein shorter or longer than the active form of the protein. The 3′ primer has the sequence 5′ GTA CGC AAG CTT GCA GGC AAA TCC AGT CTC CCT CCA GGG ATG 3′ (SEQ ID NO:10) containing the underlined Hind III restriction site followed by 30 nucleotides complementary to the 3′ end of the coding sequence of the Nodal DNA sequence in FIG. 1B.

The amplified Nodal DNA fragment and the vector pQE9 are digested with Bam HI and Hind III and the digested DNAs are then ligated together. Insertion of the Nodal DNA into the restricted pQE9 vector places the Nodal protein coding region downstream from the IPTG-inducible promoter and in-frame with an initiating AUG and the six histidine codons.

The skilled artisan appreciates that a similar approach could easily be designed and utilized to generate a pQE9-based bacterial expression construct for the expression of Lefty protein in E. coli. This would be done by designing PCR primers containing similar restriction endonuclease recognition sequences combined with gene-specific sequences for Lefty and proceeding as described above.

The ligation mixture is transformed into competent E. coli cells using standard procedures such as those described by Sambrook and colleagues (Molecular Cloning: a Laboratory Manual, 2nd Ed.; Cold Spring Harbor Laboratory Press, Cold Spring Harbor, N.Y. (1989)). E. coli strain M15/rep4, containing multiple copies of the plasmid pREP4, which expresses the lac repressor and confers kanamnycin resistance (“Kanr”), is used in carrying out the illustrative example described herein. This strain, which is only one of many that are suitable for expressing Nodal protein, is available commercially (QIAGEN, Inc., supra). Transformants are identified by their ability to grow on LB plates in the presence of ampicillin and kanamycin. Plasmid DNA is isolated from resistant colonies and the identity of the cloned DNA confirmed by restriction analysis, PCR and DNA sequencing.

Clones containing the desired constructs are grown overnight (“O/N”) in liquid culture in LB media supplemented with both ampicillin (100 μg/ml) and kanamycin (25 μg/ml). The O/N culture is used to inoculate a large culture, at a dilution of approximately 1:25 to 1:250. The cells are grown to an optical density at 600 nm (“OD600”) of between 0.4 and 0.6. Isopropyl-β-D-thiogalactopyranoside (“IPTG”) is then added to a final concentration of 1 mM to induce transcription from the lac repressor sensitive promoter, by inactivating the lacI repressor. Cells subsequently are incubated further for 3 to 4 hours. Cells then are harvested by centrifugation.

The cells are then stirred for 3-4 hours at 4° C. in 6M guanidine-HCl, pH 8. The cell debris is removed by centrifugation, and the supernatant containing the Nodal protein is loaded onto a nickel-nitrilo-tri-acetic acid (“Ni-NTA”) affinity resin column (QIAGEN, Inc., supra). Proteins with a 6×His tag bind to the Ni-NTA resin with high affinity and can be purified in a simple one-step procedure (for details see: The QIAexpressionist, 1995, QIAGEN, Inc., supra). Briefly the supernatant is loaded onto the column in 6 M guanidine-HCl, pH 8, the column is first washed with 10 volumes of 6 M guanidine-HCl, pH 8, then washed with 10 volumes of 6 M guanidine-HCl pH 6, and finally the Nodal is eluted with 6 M guanidine-HCl, pH 5.

The purified protein is then renatured by dialyzing it against phosphate-buffered saline (PBS) or 50 mM Na-acetate, pH 6 buffer plus 200 mM NaCl. Alternatively, the protein can be successfully refolded while immobilized on the Ni-NTA column. The recommended conditions are as follows: renature using a linear 6M-1M urea gradient in 500 mM NaCl, 20% glycerol, 20 mM Tris/HCl pH 7.4, containing protease inhibitors. The renaturation should be performed over a period of 1.5 hours or more. After renaturation the proteins can be eluted by the addition of 250 mM immidazole. Immidazole is removed by a final dialyzing step against PBS or 50 mM sodium acetate pH 6 buffer plus 200 mM NaCl. The purified protein is stored at 4° C. or frozen at −80° C.

The following alternative method may be used to purify Nodal expressed in E coli when it is present in the form of inclusion bodies. Unless otherwise specified, all of the following steps are conducted at 4-10° C.

Upon completion of the production phase of the E. coli fermentation, the cell culture is cooled to 4-10° C. and the cells are harvested by continuous centrifugation at 15,000 rpm (Heraeus Sepatech). On the basis of the expected yield of protein per unit weight of cell paste and the amount of purified protein required, an appropriate amount of cell paste, by weight, is suspended in a buffer solution containing 100 mM Tris, 50 mM EDTA, pH 7.4. The cells are dispersed to a homogeneous suspension using a high shear mixer.

The cells were then lysed by passing the solution through a microfluidizer (Microfuidics, Corp. or APV Gaulin, Inc.) twice at 4000-6000 psi. The homogenate is then mixed with NaCl solution to a final concentration of 0.5 M NaCl, followed by centrifugation at 7000×g for 15 min. The resultant pellet is washed again using 0.5M NaCl, 100 mM Tris, 50 mM EDTA, pH 7.4.

The resulting washed inclusion bodies are solubilized with 1.5 M guanidine hydrochloride (GuHCl) for 2-4 hours. After 7000×g centrifugation for 15 min., the pellet is discarded and the Nodal polypeptide-containing supernatant is incubated at 4° C. overnight to allow further GuHCI extraction.

Following high speed centrifugation (30,000×g) to remove insoluble particles, the GuHCl solubilized protein is refolded by quickly mixing the GuHCl extract with 20 volumes of buffer containing 50 mM sodium, pH 4.5, 150 mM NaCl, 2 mM EDTA by vigorous stirring. The refolded diluted protein solution is kept at 4° C. without mixing for 12 hours prior to further purification steps.

To clarify the refolded Nodal polypeptide solution, a previously prepared tangential filtration unit equipped with 0.16 μm membrane filter with appropriate surface area (e.g., Filtron), equilibrated with 40 mM sodium acetate, pH 6.0 is employed. The filtered sample is loaded onto a cation exchange resin (e.g., Poros HS-50, Perseptive Biosystems). The column is washed with 40 mM sodium acetate, pH 6.0 and eluted with 250 mM, 500 mM, 1000 mM, and 1500 mM NaCl in the same buffer, in a stepwise manner. The absorbance at 280 mm of the effluent is continuously monitored. Fractions are collected and further analyzed by SDS-PAGE.

Fractions containing the Nodal polypeptide are then pooled and mixed with 4 volumes of water. The diluted sample is then loaded onto a previously prepared set of tandem columns of strong anion (Poros HQ-50, Perseptive Biosystems) and weak anion (Poros CM-20, Perseptive Biosystems) exchange resins. The columns are equilibrated with 40 mM sodium acetate, pH 6.0. Both columns are washed with 40 mM sodium acetate, pH 6.0, 200 mM NaCl. The CM-20 column is then eluted using a 10 column volume linear gradient ranging from 0.2 M NaCl, 50 mM sodium acetate, pH 6.0 to 1.0 M NaCl, 50 mM sodium acetate, pH 6.5. Fractions are collected under constant A₂₈₀ monitoring of the effluent. Fractions containing the Nodal polypeptide (determined, for instance, by 16% SDS-PAGE) are then pooled.

The resultant Nodal polypeptide exhibits greater than 95% purity after the above refolding and purification steps. No major contaminant bands are observed from Commassie blue stained 16% SDS-PAGE gel when 5 μg of purified protein is loaded. The purified protein is also tested for endotoxin/LPS contamination, and typically the LPS content is less than 0.1 ng/ml according to LAL assays.

### Example 2

### Cloning and Expression of Nodal Protein in a Baculovirus Expression System

In this illustrative example, the plasmid shuttle vector pA2GP is used to insert the cloned DNA encoding the active form of the Nodal protein, lacking its naturally associated secretory signal (leader) sequence, into a baculovirus to express the active form of the Nodal protein, using a baculovirus leader and standard methods as described by Summers and colleagues (A Manual of Methods for Baculovirus Vectors and Insect Cell Culture Procedures, Texas Agricultural Experimental Station Bulletin No. 1555 (1987)). This expression vector contains the strong polyhedrin promoter of the Autographa californica nuclear polyhedrosis virus (AcMNPV) followed by the secretory signal peptide (leader) of the baculovirus gp67 protein and convenient restriction sites such as Bam HI, Xba I and Asp 718. The polyadenylation site of the simian virus 40 (“SV40”) is used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate viable virus that expresses the cloned polynucleotide.

Many other baculovirus vectors could be used in place of the vector above, such as pAc373, pVL941 and pAcIM1, as one skilled in the art would readily appreciate, as long as the construct provides appropriately located signals for transcription, translation, secretion and the like, including a signal peptide and an in-frame AUG as required. Such vectors are described, for instance, by Luckow and colleagues (Virology 170:31-39 (1989)).

The cDNA sequence encoding the mature Nodal protein in the deposited clone, lacking the AUG initiation codon and the naturally associated leader sequence shown in SEQ ID NO:2, is amplified using PCR oligonucleotide primers corresponding to the 5′ and 3′ sequences of the gene. The 5′ primer has the sequence 5′ CAA TTG GAT CCA CTT GCC AGA CAG AGA ACT CAA CTG 3′ (SEQ ID NO:11) containing the underlined Bam HI restriction enzyme site followed by 25 nucleotides of the sequence of the active form of the Nodal protein shown in SEQ ID NO:2, beginning with the indicated N-terminus of the active form of the Nodal protein. The 3′ primer has the sequence 5′ CAC TTA GGT ACC ATG TCA TCA GAG GCA CCC ACA TTC TTC 3′ (SEQ ID NO:12) containing the underlined Asp 718 restriction site followed by 27 nucleotides complementary to the 3′ coding sequence in FIG. 1B.

The skilled artisan appreciates that a similar approach could easily be designed and utilized to generate a pA2GP-based baculovirus expression construct for the expression of Lefty protein by baculovirus. This would be done by designing PCR primers containing the same, or similar, restriction endonuclease recognition sequences combined with gene-specific sequences for Lefty and proceeding as described above.

The amplified fragment is isolated from a 1% agarose gel using a commercially available kit (“Geneclean,” BIO 101 Inc., La Jolla4 Calif.). The fragment then is digested with Bam HI and Asp 718 and again is purified on a 1% agarose gel. This fragment is designated herein F1.

The plasmid is digested with the restriction enzymes Bam HI and Asp 718 and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.). This vector DNA is designated herein “V1”.

Fragment F1 and the dephosphorylated plasmid V1 are ligated together with T4 DNA ligase. E. coli HB101 or other suitable E. coli hosts such as XL-1 Blue (Stratagene Cloning Systems, La Jolla, Calif.) cells are transformed with the ligation mixture and spread on culture plates. Bacteria are identified that contain the plasmid with the human Nodal sequences by digesting DNA from individual colonies using Bam HI and Asp 718 and then analyzing the digestion product by gel electrophoresis. The sequence of the cloned fragment is confirmed by DNA sequencing. This plasmid is designated herein pA2Nodal.

Five μg of the plasmid pA2Nodal is co-transfected with 1.0 μg of a commercially available linearized baculovirus DNA (“BaculoGold™ baculovirus DNA”, Pharmingen, San Diego, Calif.), using the lipofection method described by Felgner and colleagues (Proc. Natl. Acad. Sci. USA 84:7413-7417 (1987)). One μg of BaculoGold™ virus DNA and 5 μg of the plasmid pA2Nodal are mixed in a sterile well of a microtiter plate containing 50 μl of serum-free Grace's medium (Life Technologies Inc., Gaithersburg, Md.). Afterwards, 10 μl Lipofectin plus 90 μl Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is then incubated for 5 hours at 27° C. The transfection solution is then removed from the plate and 1 ml of Grace's insect medium supplemented with 10% fetal calf serum is added. Cultivation is then continued at 27° C. for four days.

After four days the supernatant is collected and a plaque assay is performed, as described by Summers and Smith (supra). An agarose gel with “Blue Gal” (Life Technologies Inc., Gaithersburg) is used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, page 9-10). After appropriate incubation, blue stained plaques are picked with the tip of a micropipettor (e.g., Eppendorf). The agar containing the recombinant viruses is then resuspended in a emicrocentrifuge tube containing 200 μl of Grace's medium and the suspension containing the recombinant baculovirus is used to infect Sf9 cells seeded in 35 mm dishes. Four days later the supernatants of these culture dishes are harvested and then they are stored at 4° C. The recombinant virus is called V-Nodal.

To verify the expression of the active form of the Nodal protein, Sf9 cells are grown in Grace's medium supplemented with 10% heat-inactivated FBS. The cells are infected with the recombinant baculovirus V-Nodal at a multiplicity of infection (“MOI”) of about 2. If radiolabeled proteins are desired, 6 hours later the medium is removed and is replaced with SF900 II medium minus methionine and cysteine (available from Life Technologies Inc., Rockville, Md.). After 42 hours, 5 μCi of ³⁵S-methionine and 5 μCi ³⁵S-cysteine (available from Amersham) are added. The cells are further incubated for 16 hours and then are harvested by centrifugation. The proteins in the supernatant as well as the intracellular proteins are analyzed by SDS-PAGE followed by autoradiography (if radiolabeled).

Microsequencing of the amino acid sequence of the amino terminus of purified protein may be used to determine the amino terminal sequence of the active form of the Nodal protein.

### Example 3

### Cloning and Expression of Nodal in Mammalian Cells

A typical mammalian expression vector contains the promoter element, which mediates the initiation of transcription of mRNA, the protein coding sequence, and signals required for the termination of transcription and polyadenylation of the transcript. Additional elements include enhancers, Kozak sequences and intervening sequences flanked by donor and acceptor sites for RNA splicing. Highly efficient transcription can be achieved with the early and late promoters from SV40, the long terminal repeats (LTRs) from Retroviruses, e.g., RSV, HTLVI, HIVI and the early promoter of the cytomegalovirus (CMV). However, cellular elements can also be used (e.g., the human actin promoter). Suitable expression vectors for use in practicing the present invention include, for example, vectors such as pSVL and pMSG (Pharmacia, Uppsala, Sweden), pRSVcat (ATCC 37152), pSV2dhfr (ATCC 37146) and pBC12MI (ATCC 67109). Mammalian host cells that could be used include, human Hela, 293, H9 and Jurkat cells, mouse NIH3T3 and C127 cells, Cos 1, Cos 7 and CV1, quail QCl-3 cells, mouse L cells and Chinese hamster ovary (CHO) cells.

Alternatively, the gene can be expressed in stable cell lines that contain the gene integrated into a chromosome. The co-transfection with a selectable marker such as dhfr, gpt, neomycin, hygromycin allows the identification and isolation of the transfected cells.

The transfected gene can also be amplified to express large amounts of the encoded protein. The DUER (dihydrofolate reductase) marker is useful to develop cell lines that carry several hundred or even several thousand copies of the gene of interest. Another useful selection marker is the enzyme glutamine synthase (GS; Murphy, et al., Biochem J.227:277-279 (1991); Bebbington, et al., Bio/Technology 10:169-175 (1992)). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

The expression vectors pC1 and pC4 contain the strong promoter (LTR) of the Rous Sarcoma Virus (Cullen, et al., Mol. Cel. Biol. 5:438-447 (1985)) plus a fragment of the CMV-enhancer (Boshart, et al., Cell 41:521-530 (1985)). Multiple cloning sites, e.g., with the restriction enzyme cleavage sites Bam HI, Xba I and Asp 718, facilitate the cloning of the gene of interest. The vectors contain in addition the 3′ intron, the polyadenylation and termination signal of the rat preproinsulin gene.

### Example 3(a)

### Cloning and Expression in COS Cells

The expression plasmid, pNodalHA, is made by cloning a portion of the cDNA encoding the active form of the Nodal protein into the expression vector pcDNAI/Amp or pcDNAIII (which can be obtained from Invitrogen, Inc.). To produce a soluble, secreted form of the polypeptide, the active form of Nodal is fused to the secretory leader sequence of the human IL-6 gene.

The expression vector pcDNAI/amp contains: (1) an E. coli origin of replication effective for propagation in E. coli and other prokaryotic cells; (2) an ampicillin resistance gene for selection of plasmid-containing prokaryotic cells; (3) an SV40 origin of replication for propagation in eukaryotic cells; (4) a CMV promoter, a polylinker, an SV40 intron; (5) several codons encoding a hemagglutinin fragment (i.e., an t“HA” tag to facilitate purification) followed by a termination codon and polyadenylation signal arranged so that a cDNA can be conveniently placed under expression control of the CMV promoter and operably linked to the SV40 intron and the polyadenylation signal by means of restriction sites in the polylinker. The HA tag corresponds to an epitope derived from the influenza hemagglutinin protein described by Wilson and colleagues (Cell 37:767 (1984)). The fusion of the HA tag to the target protein allows easy detection and recovery of the recombinant protein with an antibody that recognizes the HA epitope. pcDNAIII contains, in addition, the selectable neomycin marker.

A DNA fragment encoding the active form of the Nodal polypeptide is cloned into the polylinker region of the vector so that recombinant protein expression is directed by the CMV promoter. The plasmid construction strategy is as follows. The Nodal cDNA of the deposited clone is amplified using primers that contain convenient restriction sites, much as described above for construction of vectors for expression of Nodal in E. coli. Suitable primers include the following, which are used in this example. The 5′ primer, containing the underlined Bam HI site, a Kozak sequence, an AUG start codon, a sequence encoding the secretory leader peptide from the human IL-6 gene, and 27 nucleotides of the 5′ coding region of the complete form of the Nodal polypeptide, has the following sequence: 5′ GCC GGA TCC GCC ACC ATG AAC TCC TTC TCC ACA AGC GCC TTC GGT CCA GTT GCC TTC TCC CTG GGG CTG CTC CTG GTG TTG CCT GCT GCC TTC CCT GCC CCA GTC ATC ACT TGC CAG ACA GAA GTC AAC TG 3′ (SEQ ID NO:13). The 3′ primer, containing the underlined Xba I and 27 of nucleotides complementary to the 3′ coding sequence immediately before the stop codon, has the following sequence: 5′ GGC TCT AGA ATG TCA TCA GAG GCA CCC ACA TTC TTC 3′ (SEQ ID NO:14).

The skilled artisan appreciates that a similar approach could easily be designed and utilized to generate a pcDNAI/amp-based eukaryotic expression construct for the expression of Lefty protein by COS cells. This would be done by designing PCR primers containing the same, or similar, restriction endonuclease recognition sequences combined with gene-specific sequences for Lefty and proceeding as described above.

The PCR amplified DNA fragment and the vector, pcDNAI/Amp, are digested with Barn HI and Xba I and then ligated. The ligation mixture is transformed into E. coli strain SURE (Stratagene Cloning Systems, La Jolla, Calif. 92037), and the transformed culture is plated on ampicillin media plates which then are incubated to allow growth of ampicillin resistant colonies. Plasmid DNA is isolated from resistant colonies and examined by restriction analysis or other means for the presence of the fragment encoding the active form of the Nodal polypeptide.

For expression of recombinant Nodal, COS cells are transfected with an expression vector, as described above, using DEAE-dextran, as described, for instance, by Sambrook and coworkers (Molecular Cloning: a Laboratory Manual, Cold Spring Laboratory Press, Cold Spring Harbor, N.Y. (1989)). Cells are incubated under conditions for expression of Nodal and Lefty by the vector.

Expression of the Nodal-HA fusion protein is detected by radiolabeling and immunoprecipitation, using methods described in, for example Harlow and colleagues (Antibodies: A Laboratory Manual, 2nd Ed.; Cold Spring Harbor Laboratory Press, Cold Spring Harbor, N.Y. (1988)). To this end, two days after transfection, the cells are labeled by incubation in media containing ³⁵S-cysteine for 8 hours. The cells and the media are collected, and the cells are washed and the lysed with detergent-containing RIPA buffer: 150 mM NaCl, 1% NP-40, 0.1% SDS, 1% NP-40, 0.5% DOC, 50 mM TRIS, pH 7.5, as described by Wilson and colleagues (supra). Proteins are precipitated from the cell lysate and from the culture media using an HA-specific monoclonal antibody. The precipitated proteins then are analyzed by SDS-PAGE and autoradiography. An expression product of the expected size is seen in the cell lysate, which is not seen in negative controls.

### Example 3(b)

### Cloning and Expression in CHO Cells

The vector pC4 is used for the expression of the active form of the Nodal polypeptide. Plasmid pC4 is a derivative of the plasmid pSV2-dhfr (ATCC Accession No. 37146). To produce a soluble, secreted form of the polypeptide, the active form of Nodal is fused to the secretory leader sequence of the human IL-6 gene. The plasmid contains the mouse DHFR gene under control of the SV40 early promoter. Chinese hamster ovary-or other cells lacking dihydrofolate activity that are transfected with these plasmids can be selected by growing the cells in a selective medium (alpha minus MEM, Life Technologies) supplemented with the chemotherapeutic agent methotrexate. The Vamplification of the DHFR genes in cells resistant to methotrexate (MTX) has been well documented (see, e.g., Alt, F. W., et al., J. Biol. Chem. 253:1357-1370 (1978); Hamlin, J. L. and Ma, C. Biochem. et Biophys. Acta, 1097:107-143 (1990); Page, M. J. and Sydenham, M. A. Biotechnology 9:64-68 (1991)). Cells grown in increasing concentrations of MTX develop resistance to the drug by overproducing the target enzyme, DHFR, as a result of amplification of the DHFR gene. If a second gene is linked to the DHFR gene, it is usually co-amplified and over-expressed. It is known in the art that this approach may be used to develop cell lines carrying more than 1,000 copies of the amplified gene(s). Subsequently, when the methotrexate is withdrawn, cell lines are obtained which contain the amplified gene integrated into one or more chromosome(s) of the host cell.

Plasmid pC4 contains for expressing the gene of interest the strong promoter of the long terminal repeat (LTR) of the Rouse Sarcoma Virus (Cullen, et al., Mol. Cell. Biol. 5:438-447 (1985)) plus a fragment isolated from the enhancer of the immediate early gene of human cytomegalovirus (CMV; Boshart, et al., Cell 41:521-530 (1985)). Downstream of the promoter are the following single restriction enzyme cleavage sites that allow the integration of the genes: Bam HI, Xba I, and Asp 718. Behind these cloning sites the plasmid contains the 3′ intron and polyadenylation site of the rat preproinsulin gene. Other high efficiency promoters can also be used for the expression, e.g., the human β-actin promoter, the SV40 early or late promoters or the long terminal repeats from other retroviruses, e.g., HIV and HTLVI. Clontech's Tet-Off and Tet-On gene expression systems and similar systems can be used to express the Nodal polypeptide in a regulated way in mammalian cells (Gossen, M., and Bujard, H. Proc. Natl. Acad. Sci. USA 89:5547-5551 (1992)). For the polyadenylation of the mRNA other signals, e.g., from the human growth hormone or globin genes can be used as well. Stable cell lines carrying a gene of interest integrated into the chromosomes can also be selected upon co-transfection with a selectable marker such as gpt, G418 or hygromycin. It is advantageous to use more than one selectable marker in the beginning, e.g., G418 plus methotrexate.

The plasmid pC4 is digested with the restriction enzymes Bam HI and Asp 718 and then dephosphorylated using calf intestinal phosphates by procedures known in the art. The vector is then isolated from a 1% agarose gel.

The DNA sequence encoding the active form of the Nodal polypeptide is amplified using PCR oligonucleotide primers corresponding to the 5′ and 3′ sequences of the desired portion of the gene. The 5′ primer containing the underlined Barn HI site, a Kozak sequence, an AUG start codon, and 26 nucleotides of the 5′ coding region of the active form of the Nodal polypeptide, has the following sequence: 5′ GAC TGG ATC CCA TAC TTG CCA GAC AGA AGT CAA CTG 3′ (SEQ ID NO:15). The 3′ primer, containing the underlined Bam HI and 26 of nucleotides complementary to the 3′ coding sequence immediately before the stop codon as shown in FIG. 1B (SEQ ID NO:1), has the following sequence: 5′ CAC TTA GGT ACC ATG TCA TCA GAG GCA CCC ACA TTC TTC 3′ (SEQ ID NO:16).

The skilled artisan appreciates that a similar approach could easily be designed and utilized to generate a pC4-based eukaryotic expression construct for the expression of Lefty protein by CHO cells. This would be done by designing PCR primers containing the same, or similar, restriction endonuclease recognition sequences combined with gene-specific sequences for Lefty and proceeding as described above.

The amplified fragment is digested with the endonucleases Bam HI and Asp 718 and then purified again on a 1% agarose gel. The isolated fragment and the dephosphorylated vector are then ligated with T4 DNA ligase. E. coli HB101 or XL-1 Blue cells are then transformed and bacteria are identified that contain the fragment inserted into plasmid pC4 using, for instance, restriction enzyme analysis.

Chinese hamster ovary cells lacking an active DHFR gene are used for transfection. Five μg of the expression plasmid pC4 is cotransfected with 0.5 μg of the plasmid pSVneo using lipofectin (Felgner, et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of metothrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 μM, 2 μM, 5 μM, 10 mM, 20 mM). The same procedure is repeated until clones are obtained which grow at a concentration of 100-200 μM. Expression of the desired gene product is analyzed, for instance, by SDS-PAGE and Western blot or by reversed phase HPLC analysis.

### Example 4

### Tissue Distribution of Nodal and Lefty mRNA Expression

Northern blot analysis is carried out to examine Nodal and Lefty gene expression in human tissues, using methods described by, among others, Sambrook and colleagues (supra). A cDNA probe containing the entire nucleotide sequence of the Nodal and/or Lefty proteins (SEQ ID NO:1) is labeled with ³²P using the rediprime™ DNA labeling system (Amersham Life Science), according to manufacturer's instructions. After labeling, the probe is purified using a NucTrap column (Stratagene, La Jolla, Calif.), according to manufacturer's protocol. The purified labeled probe is then used to examine various human tissues for Nodal and Lefty mRNA.

Multiple Tissue Northern (MTN) blots containing various human tissues (H) or human immune system tissues (IM) are obtained from Clontech and are examined with the labeled probe using ExpressHyb™ hybridization solution (Clontech) according to manufacturer's protocol number PT1190-1. Following hybridization and washing, the blots are mounted and exposed to film at −70° C. overnight, and films developed according to standard procedures.

Using a protocol such as this expression of the Nodal mRNA was detected in fetal brain, but not in most adult tissues. Furthermore, Lefty mRNA was detected in pancreas, ovary, and colon, to a lesser extent in placenta and heart, and very weakly in testes.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of all publications (including patents, patent applications, journal articles, laboratory manuals, books, or other documents) cited herein are hereby incorporated by reference.

Further, the Sequence Listing submitted herewith, and the Sequence Listing submitted with U.S. Provisional Application Serial No. 60/056,565, filed on Aug. 21, 1997 (to which the present application claims benefit of the filing date under 35 U.S.C. § 119(e)), in both computer and paper forms are hereby incorporated by reference in their entireties.

What is claimed is:

1. An isolated nucleic acid molecule nucleic acid molecule comprising a polynucleotide having a nucleotide sequence at least 95% identical to a sequence selected from the group consisting of:
(a) a nucleotide sequence encoding the Nodal polypeptide having the complete amino acid sequence in SEQ ID NO:2 (i.e., positions 1 to 283 of SEQ ID NO:2);
(b) a nucleotide sequence encoding the predicted active Nodal polypeptide having the amino acid sequence at positions 173 to 283 of SEQ ID NO:2;
(c) a nucleotide sequence encoding the Nodal polypeptide having the complete amino acid sequence encoded by the cDNA clone contained in ATCC Deposit No. 209092 or 209135;
(d) a nucleotide sequence encoding the active domain of the Nodal polypeptide having the amino acid sequence encoded by the cDNA clone contained in ATCC Deposit No. 209092 or 209135;
(e) a nucleotide sequence encoding the Lefty polypeptide having the complete amino acid sequence in SEQ ID NO:4 (i.e., positions −18 to 348 of SEQ ID NO:4);
(f) a nucleotide sequence encoding the Lefty polypeptide having the complete amino acid sequence in SEQ ID NO:4 excepting the N-terminal methionine (i.e., positions −17 to 348 of SEQ ID NO:4);
(g) a nucleotide sequence encoding the predicted active domain of the Lefty polypeptide having the amino acid sequence at positions 60 to 348 of SEQ ID NO:4;
(h) a nucleotide sequence encoding the predicted active domain of the Lefty polypeptide having the amino acid sequence at positions 118 to 348 of SEQ ID NO:4;
(i) a nucleotide sequence encoding the predicted active domain of the Lefty polypeptide having the amino acid sequence at positions 125 to 348 of SEQ ID NO:4;
(j) a nucleotide sequence encoding the Lefty polypeptide having the complete amino acid sequence encoded by the cDNA clone contained in ATCC Deposit No.209091;
(k) a nucleotide sequence encoding the Lefty polypeptide having the complete amino acid sequence excepting the N-terminal methionine encoded by the cDNA clone contained in ATCC Deposit No. 209091;
(l) a nucleotide sequence encoding the active domain of the Lefty polypeptide having the amino acid sequence encoded by the cDNA clone contained in ATCC Deposit No. 209091; and,
(m) a nucleotide sequence complementary to any of the nucleotide sequences in (a) through (l) above.

2. The nucleic acid molecule of claim 1 wherein said polynucleotide has the complete nucleotide sequence in FIGS. 1A and 1B (SEQ ID NO:1) or in FIGS. 2A and 2B (SEQ ID NO:3).

3. The nucleic acid molecule of claim I wherein said polynucleotide has the nucleotide sequence in FIGS. 1A and 1B (SEQ ID NO:1) encoding the Nodal polypeptide having the amino acid sequence in positions 1 to 283 of SEQ ID NO:2.

4. The nucleic acid molecule of claim 1 wherein said polynucleotide has the nucleotide sequence in FIGS. 2A and 2B (SEQ ID NO:3) encoding the Lefty polypeptide having the amino acid sequence in positions −18 to 348 of SEQ ID NO:4.

5. The nucleic acid molecule of claim 1 wherein said polynucleotide has the nucleotide sequence in FIGS. 1A and 1B (SEQ ID NO:1) encoding the Nodal polypeptide having the amino acid sequence in positions 2 to 283 of SEQ ID NO:2.

6. The nucleic acid molecule of claim 1 wherein said polynucleotide has the nucleotide sequence in FIGS. 2A and 2B (SEQ ID NO:3) encoding the Lefty polypeptide having the amino acid sequence in positions −17 to 348 of SEQ ID NO:4.

7. The nucleic acid molecule of claim 1 wherein said polynucleotide has the nucleotide sequence in FIGS. 1A and 1B (SEQ ID NO:1) encoding the active form of the Nodal polypeptide having the amino acid sequence from about 173 to about 283 in SEQ ID NO:2.

8. The nucleic acid molecule of claim 1 wherein said polynucleotide has the nucleotide sequence in FIGS. 2A and 2B (SEQ ID NO:3) encoding the mature form of the Lefty polypeptide having the amino acid sequence from about 1 to about 348 in SEQ ID NO:4.

9. The nucleic acid molecule of claim 1 wherein said polynucleotide has the nucleotide sequence in FIG. 2A and 2B (SEQ ID NO:3) encoding the active form of the Lefty polypeptide having the amino acid sequence from about 60 to about 348 in SEQ ID NO:4.

10. The nucleic acid molecule of claim 1 wherein said polynucleotide has the nucleotide sequence in FIGS. 2A and 2B (SEQ ID NO:3) encoding the active form of the Lefty polypeptide having the amino acid sequence from about 118 to about 348 in SEQ ID NO:4.

11. The nucleic acid molecule of claim 1 wherein said polynucleotide has the nucleotide sequence in FIGS. 2A and 2B (SEQ ID NO:3) encoding the active form of the Lefty polypeptide having the amino acid sequence from about 125 to about 348 in SEQ ID NO:4.

12. An isolated nucleic acid molecule comprising a polynucleotide having a nucleotide sequence at least 95% identical to a sequence e s elected from the group consisting of:
(a) a nucleotide sequence encoding a polypeptide comprising the amino acid sequence of residues n-283 of SEQ ID NO:2, where n is an integer in the range of 173-183;
(b) a nucleotide sequence encoding a polypeptide comprising the amino acid sequence of residues 1-m of SEQ ID NO:2, where m is an integer in the range of 249-283;
(c) a nucleotide sequence encoding a polypeptide having the amino acid sequence consisting of residues n-m of SEQ ID NO:2, where n and m are integers as defined respectively in (a) and (b) above;
(d) a nucleotide sequence encoding a polypeptide consisting of a portion of the complete Nodal amino acid sequence encoded by the cDNA clone contained in ATCC Deposit No. 209092 or 209135 wherein said portion excludes from 1 to about 182 amino acids from the amino terminus of said complete amino acid sequence encoded by the cDNA clone contained in ATCC Deposit No. 209092 or 209135;
(e) a nucleotide sequence encoding a polypeptide consisting of a portion of the complete Nodal amino acid sequence encoded by the cDNA clone contained in ATCC Deposit No. 209092 or 209135 wherein said portion excludes from 1 to about 34 amino acids from the carboxy terminus of said complete amino acid sequence encoded by the cDNA clone contained in ATCC Deposit No. 209092 or 209135; and
(f) a nucleotide sequence encoding a polypeptide consisting of a portion of the complete Nodal amino acid sequence encoded by the cDNA clone contained in ATCC Deposit No. 209092 or 209135 wherein said portion include a combination of any of the amino terminal and carboxy terminal deletions in (d) and (e), above.

13. An isolated nucleic acid molecule comprising a polynucleotide having a nucleotide sequence at least 95% identical to a sequence selected from the group consisting of:
(a) a nucleotide sequence encoding a polypeptide comprising the amino acid sequence of residues n-348 of SEQ ID NO:4, where n is an integer in the range of 1-60;
(b) a nucleotide sequence encoding a polypeptide comprising the amino acid sequence of residues n-348 of SEQ ID NO:4, where n is an integer in the range of 1-118;
(c) a nucleotide sequence encoding a polypeptide comprising the amino acid sequence of residues n-348 of SEQ ID NO:4, where n is an integer in the range of 1-125;
(d) a nucleotide sequence encoding a polypeptide comprising the amino acid sequence of residues 1-m of SEQ ID NO:4, where m is an integer in the range of 335-348;
(e) a nucleotide sequence encoding a polypeptide having the amino acid sequence consisting of residues n-m of SEQ ID NO:4, where n and m are integers as defined respectively in (a) through (d) above;
(f) a nucleotide sequence encoding a polypeptide consisting of a portion of the complete Lefty amino acid sequence encoded by the cDNA clone contained in ATCC Deposit No. 209091 wherein said portion excludes from 1 to about 78 amino acids from the amino terminus of said complete amino acid sequence encoded by the cDNA clone contained in ATCC Deposit No. 209091;
(g) a nucleotide sequence encoding a polypeptide consisting of a portion of the complete Lefty amino acid sequence encoded by the cDNA clone contained in ATCC Deposit No. 209091 wherein said portion excludes from 1 to about 136 amino acids from the amino terminus of said complete amino acid sequence encoded by the cDNA clone contained in ATCC Deposit No. 209091;
(h) a nucleotide sequence encoding a polypeptide consisting of a portion of the complete Lefty amino acid sequence encoded by the cDNA clone contained in ATCC Deposit No. 209091 wherein said portion excludes from 1 to about 143 amino acids from the amino terminus of said complete amino acid sequence encoded by the cDNA clone contained in ATCC Deposit No. 209091;
(i) a nucleotide sequence encoding a polypeptide consisting of a portion of the complete Lefty amino acid sequence encoded by the cDNA clone contained in ATCC Deposit No. 209091 wherein said portion excludes from 1 to about 13 amino acids from the carboxy terminus of said complete amino acid sequence encoded by the cDNA clone contained in ATCC Deposit No. 209091; and
(j) a nucleotide sequence encoding a polypeptide consisting of a portion of the complete Lefty amino acid sequence encoded by the cDNA clone contained in ATCC Deposit No. 209091 wherein said portion include a combination of any of the amino terminal and carboxy terminal deletions in (f) through (i), above.

14. The nucleic acid molecule of claim 1 wherein said polynucleotide has the complete nucleotide sequence of the cDNA clone contained in ATCC Deposit No. 209092, 209135 or 209091.

15. The nucleic acid molecule of claim 1 wherein said polynucleotide has the nucleotide sequence encoding the Nodal or Lefty polypeptides having the complete amino acid sequence excepting the N-terminal methionine encoded by the cDNA clones contained in ATCC Deposit No. 209092, 209135 or 209091.

16. The nucleic acid molecule of claim 1 wherein said polynucleotide has the nucleotide sequence encoding the mature form of the Lefty polypeptide having the amino acid sequence encoded by the cDNA clone contained in ATCC Deposit No. 209091.

17. The nucleic acid molecule of claim 1 wherein said polynucleotide has the nucleotide sequence encoding the active forms of the Nodal or Lefty polypeptides having the amino acid sequence encoded by the cDNA clones contained in ATCC Deposit No. 209092, 209135 or 209091.

18. An isolated nucleic acid molecule comprising a polynucleotide which hybridizes under stringent hybridization conditions to a polynucleotide having a nucleotide sequence identical to a nucleotide sequence in (a) through (m) of claim 1 wherein said polynucleotide which hybridizes does not hybridize under stringent hybridization conditions to a polynucleotide having a nucleotide sequence consisting of only A residues or of only T residues.

19. An isolated nucleic acid molecule comprising a polynucleotide which encodes the amino acid sequence of an epitope-bearing portion of a Nodal or Lefty polypeptide having an amino acid sequence in (a)through (m) of claim 1.

20. The isolated nucleic acid molecule of claim 19, which encodes an epitope-bearing portion of a Nodal polypeptide wherein the amino acid sequence of said portion is selected from the group of sequences in SEQ ID NO:2 consisting of: about Lys-54 to about Asp-62, from about Val-91 to about Leu-99, from about Lys-100 to about Gln-108, from about Cys-116 to about Pro-124, from about Gln-140 to about Leu-148, from about Trp-156 to about Ser-164, from about Arg-170, to about Gln-181, from about Cys-212 to about Phe-224, from about Tyr-239, to about Thr-247, from about Pro-251, to about Met-259, and from about Asp-263, to about His-271.

21. The isolated nucleic acid molecule of claim 19, which encodes an epitope-bearing portion of a Nodal polypeptide wherein the amino acid sequence of said portion is selected from the group of sequences in SEQ ID NO:4 consisting of: about Asp-71 to about Ser-79, from about Arg-106 to about Val-114, from about Leu-136 to about Arg-144, from about Asp-154 to about Asp-164, from about His-171 to about Asp-179, from about Gln-189 to about Leu-197, from about Pro-227 to about Glu-236, from about Gly-246 to about Glu-254, from about Pro-256 to about Gln-266, from about Cys-297 to about Ala-305, from about Ile-317 to about Pro-325, from about Ile-330 to about Val-340, and from about Val-348 to about Pro-366.

22. A recombinant vector that contains the polynucleotide of claim 1.

23. A recombinant vector that contains the polynucleotide of claim 1 operably associated with a regulatory sequence that controls gene expression.

24. A genetically engineered host cell that contains the polynucleotide of claim 1.

25. A genetically engineered host cell that contains the polynucleotide of claim 1 operatively associated with a regulatory sequence that controls gene expression.

26. A method for producing a Nodal or Lefty polypeptide, comprising;
(a) culturing the genetically engineered host cell of claim 25 under conditions suitable to produce the polypeptide; and
(b) recovering said polypeptide.

27. An isolated Nodal and Lefty polypeptide comprising an amino acid sequence at least 95% identical to a sequence selected from the group consisting of:
(a) the amino acid sequence of the full-length Nodal polypeptide having the complete amino acid sequence shown in SEQ ID NO:2 (i.e., positions 1 to 283 of SEQ ID NO:2);
(b) the amino acid sequence of the predicted active Nodal polypeptide having the amino acid sequence at positions 173 to 283 of SEQ ID NO:2;
(c) the amino acid sequence of the Nodal polypeptide having the complete amino acid sequence encoded by the cDNA clone contained in ATCC Deposit No. 209092 or 209135;
(d) the amino acid sequence of the active domain of the Nodal polypeptide having the amino acid sequence encoded by the cDNA clone contained in ATCC Deposit No. 209092 or 209135;
(e) the amino acid sequence of the Lefty polypeptide having the complete amino acid sequence in SEQ ID NO:4 (i.e., positions −18 to 348 of SEQ ID NO:4);
(f) the amino acid sequence of the Lefty polypeptide having the complete amino acid sequence in SEQ ID NO:4 excepting the N-terminal methionine (i.e., positions −17 to 348 of SEQ ID NO:4);
(g) the amino acid sequence of the predicted active domain of the Lefty polypeptide having the amino acid sequence at positions 60 to 348 of SEQ ID NO:4;
(h) the amino acid sequence of the predicted active domain of the Lefty polypeptide having the amino acid sequence at positions 118 to 348 of SEQ ID NO:4;
(i) the amino acid sequence of the predicted active domain of the Lefty polypeptide having the amino acid sequence at positions 125 to 348 of SEQ ID NO:4;
(j) the amino acid sequence of the Lefty polypeptide having the complete amino acid sequence encoded by the cDNA clone contained in ATCC Deposit No.209091;
(k) the amino acid sequence of the Lefty polypeptide having the complete amino acid sequence excepting the N-terminal methionine encoded by the cDNA clone contained in ATCC Deposit No. 209091, and;
(l) the amino acid sequence of the active domain of the Lefty polypeptide having the amino acid sequence encoded by the cDNA clone contained in ATCC Deposit No. 209091.

28. An isolated polypeptide comprising an epitope-bearing portion of the Nodal protein, wherein said portion is selected from the group consisting of: a polypeptide comprising amino acid residues from about Lys-54 to about Asp-62 of SEQ ID NO:2, a polypeptide comprising amino acid residues from about Val-91 to about Leu-99 of SEQ ID NO:2, a polypeptide comprising amino acid residues from about Lys-100 to about Gln-108 of SEQ ID NO:2, a polypeptide comprising amino acid residues from about Cys-116 to about Pro-124 of SEQ ID NO:2, a polypeptide comprising amino acid residues from about Gln-140 to about Leu-148 of SEQ ID NO:2, a polypeptide comprising amino acid residues from about Trp-156 to about Ser-164 of SEQ ID NO:2, a polypeptide comprising amino acid residues from about Arg-170 to about Gln-181 of SEQ ID NO:2, a polypeptide comprising amino acid residues from about Cys-212 to about Phe-224 of SEQ ID NO:2, a polypeptide comprising amino acid residues from about Tyr-239 to about Thr-247 of SEQ ID NO:2, a polypeptide comprising amino acid residues from about Pro-251 to about Met-259 of SEQ ID NO:2, and a polypeptide comprising amino acid residues from about Asp-263 to about His-271 of SEQ ID NO:2.

29. An isolated polypeptide comprising an epitope-bearing portion of the Lefty protein, wherein said portion is selected from the group consisting of: a polypeptide comprising amino acid residues from about Asp-71 to about Ser-79 of SEQ ID NO:4, a polypeptide comprising amino acid residues from about Arg-106 to about Val-114 of SEQ ID NO:4, a polypeptide comprising amino acid residues from about Leu-136 to about Arg-144 of SEQ ID NO:4, a polypeptide comprising amino acid residues from about Asp-154 to about Asp-164 of SEQ ID NO:4, a polypeptide comprising amino acid residues from about His-171 to about Asp-179 of SEQ ID NO:4, a polypeptide comprising amino acid residues from about Gln-189 to about Leu-197 of SEQ ID NO:4, a polypeptide comprising amino acid residues from about Pro-227 to about Glu-236 of SEQ ID NO:4, a polypeptide comprising amino acid residues from about Gly-246 to about Glu-254 of SEQ ID NO:4, a polypeptide comprising amino acid residues from about Pro-256 to about Gln-266 of SEQ ID NO:4, from about Cys-297 to about Ala-305 of SEQ ID NO:4, a polypeptide comprising amino acid residues from about Ile-317 to about Pro-325 of SEQ ID NO:4, a polypeptide comprising amino acid residues from about Ile-330 to about Val-340 of SEQ ID NO:4, and a polypeptide comprising amino acid residues from about Val-348 to about Pro-366 of SEQ ID NO:4.

30. An isolated antibody that binds specifically to a Nodal and Lefty polypeptide of claim 27.

31. An isolated nucleic acid molecule comprising a polynucleotide having a sequence at least 95% identical to a sequence selected from the group consisting of:
(a) the nucleotide sequence of SEQ ID NO:7);
(b) the nucleotide sequence of SEQ ID NO:8);
(c) the nucleotide sequence of a portion of the sequence shown in FIGS. 1A and 1B (SEQ ID NO:1) wherein said portion comprises at least 50 contiguous nucleotides from nucleotide 1 to nucleotide 1130;
(d) the nucleotide sequence of a portion of the sequence shown in FIGS. 1A and 1B (SEQ ID NO:1) wherein said portion consists of nucleotides 250-1130, 500-1130, 750-1130, 1000-1130, 1-1000, 250-1000, 500-1000, 750-1000, 1-750, 250-750, 500-750, 1-500, 250-500, and 1-250 of SEQ ID NO:1;
(e) the nucleotide sequence of a portion of the sequence shown in FIGS. 2A and 2B (SEQ ID NO:3) wherein said portion comprises at least 50 contiguous nucleotides from nucleotide 1 to 950 and 1150 to 1688;
(f) the nucleotide sequence of a portion of the sequence shown in FIGS. 2A and 2B (SEQ ID NO:3) wherein said portion consists of nucleotides 250-1688, 500-1688, 750-1688, 1000-1688, 1250-1688, 1500-1688, 1-1500, 250-1500, 500-1500, 750-1500, 1000-1500, 1250-1500, 1-1250, 250-1250, 500-1250, 750-1250, 1000-1250, 1-1000, 250-1000, 500-1000, 750-1000, 1-750, 250-750, 500-750, 1-500, and 250-500 of SEQ ID NO:3; and
(g) a nucleotide sequence complementary to any of the nucleotide sequences in (a) through (f) above.

32. A method for preventing, treating, or ameliorating a medical condition which comprises administering to a mammalian subject a therapeutically effective amount of the polypeptide of claim 27.

33. A method for preventing, treating, or ameliorating a medical condition which comprises administering to a mammalian subject a therapeutically effective amount of the polynucleotide of claim 1.

34. A method of diagnosing a pathological condition or a susceptibility to a pathological condition in a subject related to expression or activity of Nodal or Lefty comprising:
(a) determining the presence or absence of a mutation in the polynucleotide of claim 1;
(b) diagnosing a pathological condition or a susceptibility to a pathological condition based on the presence or absence of said mutation.

35. A method of diagnosing a pathological condition or a susceptibility to a pathological condition in a subject related to expression or activity of Nodal or Lefty comprising:
(a) determining the presence or amount of expression of the polypeptide of claim 27 in a biological sample;
(b) diagnosing a pathological condition or a susceptibility to a pathological condition based on the presence or amount of expression of the polypeptide.

36. A method of identifying compounds capable of enhancing or inhibiting a Nodal or Lefty activity comprising:
(a) contacting the polypeptide of claim 27, with a candidate compound; and
(b) assaying for activity.
